SEQUENCE LISTING (70 sequences)

SEQ ID NO: 1 - DNA, 16 bp, synthetic construct
  source 1..16: mol_type = other DNA
  modified_base 1: mod_base = OTHER; note = LNA guanine
  modified_base 2: mod_base = OTHER; note = LNA cytosine
  misc_feature 1..16: note = phosphothionate-modified backbone between every residue
  modified_base 15: mod_base = OTHER; note = LNA cytosine
  modified_base 16: mod_base = OTHER; note = LNA cytosine
gccatcgcgggtagcc

SEQ ID NO: 2 - DNA, 16 bp, synthetic construct
  source 1..16: mol_type = other DNA
  modified_base 1: mod_base = OTHER; note = LNA guanosine
  modified_base 2: mod_base = OTHER; note = LNA cytosine
  modified_base 15: mod_base = OTHER; note = LNA cytosine
  modified_base 16: mod_base = OTHER; note = LNA cytosine
  misc_feature 1: note = 5' amino-carbon 6 chain
  misc_feature 1..16: note = phosphothionate-modifined backbone between every residue
gccatcgcgggtagcc

SEQ ID NO: 3 - DNA, 16 bp, synthetic construct
  source 1..16: mol_type = other DNA
  misc_feature 1..16: note = phosphothionate-modified backbone between each residue
  modified_base 1: mod_base = OTHER; note = LNA thymine
  modified_base 2: mod_base = OTHER; note = LNA guanine
  modified_base 15: mod_base = OTHER; note = LNA thymine
  modified_base 16: mod_base = OTHER; note = LNA cytosine
  misc_feature 1: note = 5' amino-carbon 6 chain
tgtcgggagggccatc

SEQ ID NO: 4 - DNA, 16 bp, synthetic construct
  source 1..16: mol_type = other DNA
  misc_feature 1: note = 5- amino-carbon 6 chain
  misc_feature 3..14: note = phosphothionate-modified backbone between every residue
  modified_base 1: mod_base = OTHER; note = LNA thymine
  modified_base 2: mod_base = OTHER; note = LNA guanine
  modified_base 15: mod_base = OTHER; note = LNA thymine
  modified_base 16: mod_base = OTHER; note = LNA cytosine
tgtcgggagggccatc

SEQ ID NO: 5 - DNA, 16 bp, synthetic construct
  source 1..16: mol_type = other DNA
  misc_feature 1: note = 5' amino-carbon 6 chain
  modified_base 1: mod_base = OTHER; note = LNA thymine
  modified_base 2: mod_base = OTHER; note = LNA guanine
  modified_base 15: mod_base = OTHER; note = LNA thymine
  modified_base 16: mod_base = OTHER; note = LNA cytosine
  misc_feature 1..2: note = methylphosphonate-modified backbone
  misc_feature 3..14: note = phosphothionate-modified backbone between every residue
  misc_feature 15..16: note = methylphosphonate-modified backbone
tgtcgggagggccatc

SEQ ID NO: 6 - DNA, 16 bp, synthetic construct
  source 1..16: mol_type = other DNA
  misc_feature 1: note = 5' amino-carbon 6 chain
  modified_base 1: mod_base = OTHER; note = BNA thymine
  modified_base 2: mod_base = OTHER; note = BNA guanine
  modified_base 15: mod_base = OTHER; note = BNA thymine
  modified_base 16: mod_base = OTHER; note = BNA cytosine
  misc_feature 1..16: note = phosphothionate-modified backbone between every residue
tgtcgggagggccatc

SEQ ID NO: 7 - DNA, 16 bp, synthetic construct
  source 1..16: mol_type = other DNA
  misc_feature 1..16: note = phosphothionate-modified backbone between every residue
  modified_base 1: mod_base = OTHER; note = LNA guanine
  modified_base 2: mod_base = OTHER; note = LNA cytosine
  modified_base 15: mod_base = OTHER; note = LNA cytosine
  modified_base 16: mod_base = OTHER; note = LNA cytosine
gccatcgcgggtagcc

SEQ ID NO: 8 - DNA, 16 bp, synthetic construct
  source 1..16: mol_type = other DNA
  modified_base 1: mod_base = OTHER; note = LNA thymine
  modified_base 2: mod_base = OTHER; note = LNA cytosine
  modified_base 15: mod_base = OTHER; note = LNA cytosine
  modified_base 16: mod_base = OTHER; note = LNA guanine
  misc_feature 1..16: note = phosphothionate-modified backbone between every residue
tccaaacgagtctccg

SEQ ID NO: 9 - DNA, 16 bp, synthetic construct
  source 1..16: mol_type = other DNA
  misc_feature 1: note = 5' amino-carbon chain
  misc_feature 1..16: note = phosphothionate-modified backbone between every residue
  modified_base 1: mod_base = OTHER; note = LNA thymine
  modified_base 2: mod_base = OTHER; note = LNA cytosine
  modified_base 15: mod_base = OTHER; note = LNA cytosine
  modified_base 16: mod_base = OTHER; note = LNA guanine
tccaaacgagtctccg

SEQ ID NO: 10 - DNA, 16 bp, synthetic construct
  source 1..16: mol_type = other DNA
  misc_feature 1..16: note = phosphothionate-modified backbone between every residue
  modified_base 1: mod_base = OTHER; note = LNA guanine
  modified_base 2: mod_base = OTHER; note = LNA adenine
  modified_base 15: mod_base = OTHER; note = LNA adenine
  modified_base 16: mod_base = OTHER; note = LNA thymine
gattctgaaaccagat

SEQ ID NO: 11 - DNA, 16 bp, synthetic construct
  source 1..16: mol_type = other DNA
  misc_feature 1: note = 5' amino-carbon 6 chain
  misc_feature 1..16: note = phosphothionate-modified backbone between every residue
  modified_base 1: mod_base = OTHER; note = LNA guanine
  modified_base 2: mod_base = OTHER; note = LNA adenine
  modified_base 15: mod_base = OTHER; note = LNA adenine
  modified_base 16: mod_base = OTHER; note = LNA thymine
gattctgaaaccagat

SEQ ID NO: 12 - DNA, 16 bp, synthetic construct
  source 1..16: mol_type = other DNA
  misc_feature 1..16: note = phosphothionate-modified backbone between every residue
  modified_base 1: mod_base = OTHER; note = LNA guanine
  modified_base 2: mod_base = OTHER; note = LNA cytosine
  modified_base 15: mod_base = OTHER; note = LNA adenine
  modified_base 16: mod_base = OTHER; note = LNA cytosine
gcgggcgccctgccac

SEQ ID NO: 13 - DNA, 16 bp, synthetic construct
  source 1..16: mol_type = other DNA
  misc_feature 1: note = 5' amino-carbon 6 chain
  misc_feature 1..16: note = phosphothionate-modified backbone between every residue
  modified_base 1: mod_base = OTHER; note = LNA guanine
  modified_base 2: mod_base = OTHER; note = LNA cytosine
  modified_base 15: mod_base = OTHER; note = LNA adenine
  modified_base 16: mod_base = OTHER; note = LNA cytosine
gcgggcgccctgccac

SEQ ID NO: 14 - DNA, 16 bp, synthetic construct
  source 1..16: mol_type = other DNA
  misc_feature 1..16: note = phosphothionate-modified backbone between every residue
  modified_base 1: mod_base = OTHER; note = LNA thymine
  modified_base 2: mod_base = OTHER; note = LNA cytosine
  modified_base 15: mod_base = OTHER; note = LNA guanine
  modified_base 16: mod_base = OTHER; note = LNA cytosine
tcatccagcagcaggc

SEQ ID NO: 15 - DNA, 16 bp, synthetic construct
  source 1..16: mol_type = other DNA
  misc_feature 1: note = 5' amino-carbon 6 chain
  misc_feature 1..16: note = phosphothionate-modified backbone between every residue
  modified_base 1: mod_base = OTHER; note = LNA thymine
  modified_base 2: mod_base = OTHER; note = LNA cytosine
  modified_base 15: mod_base = OTHER; note = LNA guanine
  modified_base 16: mod_base = OTHER; note = LNA cytosine
tcatccagcagcaggc

SEQ ID NO: 16 - DNA, 16 bp, synthetic construct
  source 1..16: mol_type = other DNA
  misc_feature 1..16: note = phosphothionate-modified backbone
  modified_base 1: mod_base = OTHER; note = LNA thymine
  modified_base 2: mod_base = OTHER; note = LNA adenine
  modified_base 15..16: mod_base = OTHER; note = LNA thymine
tagccagccaggtgtt

SEQ ID NO: 17 - DNA, 16 bp, synthetic construct
  source 1..16: mol_type = other DNA
  misc_feature 1: note = 5' amino-carbon 6 chain
  misc_feature 1..16: note = phosphothionate-modified backbone between every residue
  modified_base 1: mod_base = OTHER; note = LNA thymine
  modified_base 2: mod_base = OTHER; note = LNA adenine
  modified_base 15..16: mod_base = OTHER; note = LNA thymine
tagccagccaggtgtt

SEQ ID NO: 18 - DNA, 16 bp, synthetic construct
  source 1..16: mol_type = other DNA
  misc_feature 1..16: note = phosphothionate-modified backbone between every residue
  modified_base 1: mod_base = OTHER; note = LNA cytosine
  modified_base 2: mod_base = OTHER; note = LNA adenine
  modified_base 15: mod_base = OTHER; note = LNA guanine
  modified_base 16: mod_base = OTHER; note = LNA guanine
cagcgtcggaaggtgg

SEQ ID NO: 19 - DNA, 16 bp, synthetic construct
  source 1..16: mol_type = other DNA
  misc_feature 1..16: note = phosphothionate-modified backbone between every residue
  misc_feature 1: note = 5' amino-carbon 6 chain
  modified_base 1: mod_base = OTHER; note = LNA cytosine
  modified_base 2: mod_base = OTHER; note = LNA adenine
  modified_base 15: mod_base = OTHER; note = LNA guanine
  modified_base 16: mod_base = OTHER; note = LNA guanine
cagcgtcggaaggtgg

SEQ ID NO: 20 - DNA, 16 bp, synthetic construct
  source 1..16: mol_type = other DNA
  misc_feature 1..16: note = phosphothionate-modified backbone between every residue
  modified_base 1: mod_base = OTHER; note = LNA thymine
  modified_base 2: mod_base = OTHER; note = LNA adenine
  modified_base 15: mod_base = OTHER; note = LNA adenine
  modified_base 16: mod_base = OTHER; note = LNA guanine
tagacagcgtcggaag

SEQ ID NO: 21 - DNA, 16 bp, synthetic construct
  source 1..16: mol_type = other DNA
  misc_feature 1..16: note = phosphothionate-modified backbone between every residue
  misc_feature 1: note = 5' amino-carbon 6 chain
  modified_base 1: mod_base = OTHER; note = LNA thymine
  modified_base 2: mod_base = OTHER; note = LNA adenine
  modified_base 15: mod_base = OTHER; note = LNA adenine
  modified_base 16: mod_base = OTHER; note = LNA guanine
tagacagcgtcggaag

SEQ ID NO: 22 - DNA, 16 bp, synthetic construct
  source 1..16: mol_type = other DNA
  misc_feature 1..16: note = phosphothionate-modified backbone between every residue
  modified_base 1: mod_base = OTHER; note = LNA adenine
  modified_base 2: mod_base = OTHER; note = LNA thymine
  modified_base 15: mod_base = OTHER; note = LNA guanine
  modified_base 16: mod_base = OTHER; note = LNA adenine
ataggatccacaggga

SEQ ID NO: 23 - DNA, 16 bp, synthetic construct
  source 1..16: mol_type = other DNA
  misc_feature 1..16: note = phosphothionate-modified backbone between every residue
  misc_feature 1: note = 5' amino-carbon 6 chain
  modified_base 1: mod_base = OTHER; note = LNA adenine
  modified_base 2: mod_base = OTHER; note = LNA thymine
  modified_base 15: mod_base = OTHER; note = LNA guanine
  modified_base 16: mod_base = OTHER; note = LNA adenine
ataggatccacaggga

SEQ ID NO: 24 - DNA, 16 bp, synthetic construct
  source 1..16: mol_type = other DNA
  misc_feature 1..16: note = phosphothionate-modified backbone between every residue
  misc_feature 1: note = 5' amino-carbon 6 chain
  modified_base 1: mod_base = OTHER; note = BNA adenine
  modified_base 2: mod_base = OTHER; note = BNA thymine
  modified_base 15: mod_base = OTHER; note = BNA guanine
  modified_base 16: mod_base = OTHER; note = BNA adenine
ataggatccacaggga

SEQ ID NO: 25 - DNA, 16 bp, synthetic construct
  source 1..16: mol_type = other DNA
  misc_feature 1..16: note = phosphothionate-modified backbone between every residue
  modified_base 1: mod_base = OTHER; note = LNA thymine
  modified_base 2: mod_base = OTHER; note = LNA cytosine
  modified_base 15: mod_base = OTHER; note = LNA adenine
  modified_base 16: mod_base = OTHER; note = LNA guanine
tctataggatccacag

SEQ ID NO: 26 - DNA, 16 bp, synthetic construct
  source 1..16: mol_type = other DNA
  misc_feature 1..16: note = phosphothionate-modified backbone between every residue
  misc_feature 1: note = 5' amino-carbon 6 chain
  modified_base 1: mod_base = OTHER; note = LNA thymine
  modified_base 2: mod_base = OTHER; note = LNA cytosine
  modified_base 15: mod_base = OTHER; note = LNA adenine
  modified_base 16: mod_base = OTHER; note = LNA guanine
tctataggatccacag

SEQ ID NO: 27 - DNA, 16 bp, synthetic construct
  source 1..16: mol_type = other DNA
  misc_feature 1..16: note = phosphothionate-modified backbone between every residue
  modified_base 1: mod_base = OTHER; note = LNA guanine
  modified_base 2: mod_base = OTHER; note = LNA cytosine
  modified_base 15: mod_base = OTHER; note = LNA guanine
  modified_base 16: mod_base = OTHER; note = LNA guanine
gcactaatcatccagg

SEQ ID NO: 28 - DNA, 16 bp, synthetic construct
  source 1..16: mol_type = other DNA
  misc_feature 1..16: note = phosphothionate-modified backbone between every residue
  misc_feature 1: note = 5' amino-carbon 6 chain
  modified_base 1: mod_base = OTHER; note = LNA guanine
  modified_base 2: mod_base = OTHER; note = LNA cytosine
  modified_base 15: mod_base = OTHER; note = LNA guanine
  modified_base 16: mod_base = OTHER; note = LNA guanine
gcactaatcatccagg

SEQ ID NO: 29 - DNA, 15 bp, synthetic construct
  source 1..15: mol_type = other DNA
  misc_feature 1..15: note = phosphothionate-modified backbone between every residue
  modified_base 1: mod_base = OTHER; note = LNA cytosine
  modified_base 2: mod_base = OTHER; note = LNA adenine
  modified_base 3: mod_base = OTHER; note = LNA guanine
  modified_base 13: mod_base = OTHER; note = LNA guanine
  modified_base 14: mod_base = OTHER; note = LNA thymine
  modified_base 15: mod_base = OTHER; note = LNA guanine
cagcgtcggaaggtg

SEQ ID NO: 30 - DNA, 15 bp, synthetic construct
  source 1..15: mol_type = other DNA
  misc_feature 1..15: note = phosphothionate-modified backbone between every residue
  misc_feature 1: note = 5' amino-carbon 6 chain
  modified_base 1: mod_base = OTHER; note = LNA cytosine
  modified_base 2: mod_base = OTHER; note = LNA adenine
  modified_base 3: mod_base = OTHER; note = LNA guanine
  modified_base 13: mod_base = OTHER; note = LNA guanine
  modified_base 14: mod_base = OTHER; note = LNA thymine
  modified_base 15: mod_base = OTHER; note = LNA guanine
cagcgtcggaaggtg

SEQ ID NO: 31 - DNA, 17 bp, synthetic construct
  source 1..17: mol_type = other DNA
  misc_feature 1..17: note = phosphothionate-modified backbone between every residue
  modified_base 1: mod_base = OTHER; note = 2'-methyloxyethyl cytosine
  modified_base 2: mod_base = OTHER; note = 2'-methyloxyethyl cytosine
  modified_base 3: mod_base = OTHER; note = 2'-methyloxyethyl adenine
  modified_base 4: mod_base = OTHER; note = 2'-methyloxyethyl guanine
  modified_base 14: mod_base = OTHER; note = 2'-methyloxyethyl guanine
  modified_base 15: mod_base = OTHER; note = 2'-methyloxyethyl thymine
  modified_base 16: mod_base = OTHER; note = 2'-methyloxyethyl guanine
  modified_base 17: mod_base = OTHER; note = 2'-methyloxyethyl thymine
ccagcgtcggaaggtgt

SEQ ID NO: 32 - DNA, 17 bp, synthetic construct
  source 1..17: mol_type = other DNA
  misc_feature 1..17: note = phosphothionate-modified backbone between every residue
  misc_feature 1: note = 5' amino-carbon 6 chain
  modified_base 1: mod_base = OTHER; note = 2'-methyloxyethyl cytosine
  modified_base 2: mod_base = OTHER; note = 2'-methyloxyethyl cytosine
  modified_base 3: mod_base = OTHER; note = 2'-methyloxyethyl adenine
  modified_base 4: mod_base = OTHER; note = 2'-methyloxyethyl guanine
  modified_base 14: mod_base = OTHER; note = 2'-methyloxyethyl guanine
  modified_base 15: mod_base = OTHER; note = 2'-methyloxyethyl thymine
  modified_base 16: mod_base = OTHER; note = 2'-methyloxyethyl guanine
  modified_base 17: mod_base = OTHER; note = 2'-methyloxyethyl thymine
ccagcgtcggaaggtgt

SEQ ID NO: 33 - DNA, 20 bp, synthetic construct
  source 1..20: mol_type = other DNA
  misc_feature 1..20: note = phosphothionate-modified backbone between every residue
  modified_base 1: mod_base = OTHER; note = LNA cytosine
  modified_base 2: mod_base = OTHER; note = LNA cytosine
  modified_base 4: mod_base = OTHER; note = LNA adenine
  modified_base 15: mod_base = OTHER; note = LNA guanine
  modified_base 16: mod_base = OTHER; note = LNA adenine
  modified_base 19: mod_base = OTHER; note = LNA guanine
  modified_base 20: mod_base = OTHER; note = LNA thymine
cctagacagcgtcggaaggt

SEQ ID NO: 34 - DNA, 18 bp, synthetic construct
  source 1..18: mol_type = other DNA
  misc_feature 1..18: note = phosphothionate-modified backbone between every residue
  modified_base 1: mod_base = OTHER; note = LNA adenine
  modified_base 2: mod_base = OTHER; note = LNA thymine
  modified_base 3: mod_base = OTHER; note = LNA adenine
  modified_base 16: mod_base = OTHER; note = LNA adenine
  modified_base 17: mod_base = OTHER; note = LNA guanine
  modified_base 18: mod_base = OTHER; note = LNA guanine
ataggatccacagggagg

SEQ ID NO: 35 - DNA, 18 bp, synthetic construct
  source 1..18: mol_type = other DNA
  misc_feature 1..18: note = phosphothionate-modified backbone between every residue
  modified_base 1: mod_base = OTHER; note = LNA cytosine
  modified_base 2: mod_base = OTHER; note = LNA guanine
  modified_base 3: mod_base = OTHER; note = LNA guanine
  modified_base 16: mod_base = OTHER; note = LNA cytosine
  modified_base 17: mod_base = OTHER; note = LNA guanine
  modified_base 18: mod_base = OTHER; note = LNA guanine
cggctctgggatccccgg

SEQ ID NO: 36 - DNA, 17 bp, synthetic construct
  source 1..17: mol_type = other DNA
  misc_feature 1..17: note = phosphothionate-modified backbone between every residue
  modified_base 1: mod_base = OTHER; note = LNA guanine
  modified_base 2: mod_base = OTHER; note = LNA guanine
  modified_base 3: mod_base = OTHER; note = LNA guanine
  modified_base 15: mod_base = OTHER; note = LNA cytosine
  modified_base 16: mod_base = OTHER; note = LNA cytosine
  modified_base 17: mod_base = OTHER; note = LNA cytosine
ggggcggagacacgccc

SEQ ID NO: 37 - DNA, 18 bp, synthetic construct
  source 1..18: mol_type = other DNA
  misc_feature 1..18: note = phosphothionate-modified backbone between every residue
  modified_base 1: mod_base = OTHER; note = LNA adenine
  modified_base 2: mod_base = OTHER; note = LNA guanine
  modified_base 3: mod_base = OTHER; note = LNA adenine
  modified_base 16: mod_base = OTHER; note = LNA cytosine
  modified_base 17: mod_base = OTHER; note = LNA adenine
  modified_base 18: mod_base = OTHER; note = LNA guanine
agaaggcaggaatcccag

SEQ ID NO: 38 - DNA, 18 bp, synthetic construct
  source 1..18: mol_type = other DNA
  misc_feature 1..18: note = phosphothionate-modified backbone between every residue
  modified_base 1: mod_base = OTHER; note = LNA guanine
  modified_base 2: mod_base = OTHER; note = LNA cytosine
  modified_base 3: mod_base = OTHER; note = LNA adenine
  modified_base 16: mod_base = OTHER; note = LNA cytosine
  modified_base 17: mod_base = OTHER; note = LNA guanine
  modified_base 18: mod_base = OTHER; note = LNA guanine
gcaggaatcccaggccgg

SEQ ID NO: 39 - DNA, 18 bp, synthetic construct
  source 1..18: mol_type = other DNA
  misc_feature 1..18: note = phosphothionate-modified backbone between every residue
  modified_base 1: mod_base = OTHER; note = LNA guanine
  modified_base 2: mod_base = OTHER; note = LNA guanine
  modified_base 3: mod_base = OTHER; note = LNA adenine
  modified_base 16: mod_base = OTHER; note = LNA guanine
  modified_base 17: mod_base = OTHER; note = LNA cytosine
  modified_base 18: mod_base = OTHER; note = LNA cytosine
ggagtctctcaccgggcc

SEQ ID NO: 40 - DNA, 18 bp, synthetic construct
  source 1..18: mol_type = other DNA
  misc_feature 1..18: note = phosphothionate-modified backbone between every residue
  modified_base 1: mod_base = OTHER; note = LNA adenine
  modified_base 2: mod_base = OTHER; note = LNA guanine
  modified_base 3: mod_base = OTHER; note = LNA adenine
  modified_base 16: mod_base = OTHER; note = LNA cytosine
  modified_base 17: mod_base = OTHER; note = LNA cytosine
  modified_base 18: mod_base = OTHER; note = LNA cytosine
agaggccagcgagctccc

SEQ ID NO: 41 - DNA, 18 bp, synthetic construct
  source 1..18: mol_type = other DNA
  misc_feature 1..18: note = phosphothionate-modified backbone between every residue
  modified_base 1: mod_base = OTHER; note = LNA guanine
  modified_base 2: mod_base = OTHER; note = LNA guanine
  modified_base 3: mod_base = OTHER; note = LNA cytosine
  modified_base 16: mod_base = OTHER; note = LNA guanine
  modified_base 17: mod_base = OTHER; note = LNA guanine
  modified_base 18: mod_base = OTHER; note = LNA guanine
ggctctgggatccccggg

SEQ ID NO: 42 - DNA, 18 bp, synthetic construct
  source 1..18: mol_type = other DNA
  misc_feature 1..18: note = phosphothionate-modified backbone between every residue
  modified_base 1: mod_base = OTHER; note = LNA cytosine
  modified_base 2: mod_base = OTHER; note = LNA adenine
  modified_base 3: mod_base = OTHER; note = LNA guanine
  modified_base 16: mod_base = OTHER; note = LNA guanine
  modified_base 17: mod_base = OTHER; note = LNA cytosine
  modified_base 18: mod_base = OTHER; note = LNA thymine
cagagaggccagcgagct

SEQ ID NO: 43 - DNA, 18 bp, synthetic construct
  source 1..18: mol_type = other DNA
  misc_feature 1..18: note = phosphothionate-modified backbone between every residue
  modified_base 1: mod_base = OTHER; note = LNA guanine
  modified_base 2: mod_base = OTHER; note = LNA adenine
  modified_base 3: mod_base = OTHER; note = LNA cytosine
  modified_base 16: mod_base = OTHER; note = LNA thymine
  modified_base 17: mod_base = OTHER; note = LNA guanine
  modified_base 18: mod_base = OTHER; note = LNA guanine
gacagcgtcggaaggtgg

SEQ ID NO: 44 - DNA, 18 bp, synthetic construct
  source 1..18: mol_type = other DNA
  misc_feature 1..18: note = phosphothionate-modified backbone between every residue
  modified_base 1: mod_base = OTHER; note = LNA guanine
  modified_base 2: mod_base = OTHER; note = LNA thymine
  modified_base 3: mod_base = OTHER; note = LNA adenine
  modified_base 16: mod_base = OTHER; note = LNA guanine
  modified_base 17: mod_base = OTHER; note = LNA thymine
  modified_base 18: mod_base = OTHER; note = LNA thymine
gtaactctaatccaggtt

SEQ ID NO: 45 - DNA, 18 bp, synthetic construct
  source 1..18: mol_type = other DNA
  misc_feature 1..18: note = phosphothionate-modified backbone between every residue
  modified_base 1: mod_base = OTHER; note = LNA adenine
  modified_base 2: mod_base = OTHER; note = LNA cytosine
  modified_base 3: mod_base = OTHER; note = LNA adenine
  modified_base 16: mod_base = OTHER; note = LNA guanine
  modified_base 17: mod_base = OTHER; note = LNA guanine
  modified_base 18: mod_base = OTHER; note = LNA cytosine
acaagggcacagagaggc

SEQ ID NO: 46 - DNA, 18 bp, synthetic construct
  source 1..18: mol_type = other DNA
  misc_feature 1..18: note = phosphothionate-modified backbone between every residue
  modified_base 1: mod_base = OTHER; note = LNA guanine
  modified_base 2: mod_base = OTHER; note = LNA adenine
  modified_base 3: mod_base = OTHER; note = LNA guanine
  modified_base 16: mod_base = OTHER; note = LNA thymine
  modified_base 17: mod_base = OTHER; note = LNA cytosine
  modified_base 18: mod_base = OTHER; note = LNA adenine
gagctcccttgcacgtca

SEQ ID NO: 47 - DNA, 17 bp, synthetic construct
  source 1..17: mol_type = other DNA
  misc_feature 1..17: note = phosphothionate-modified backbone between every residue
  modified_base 1: mod_base = OTHER; note = LNA cytosine
  modified_base 2: mod_base = OTHER; note = LNA cytosine
  modified_base 3: mod_base = OTHER; note = LNA guanine
  modified_base 15: mod_base = OTHER; note = LNA guanine
  modified_base 16: mod_base = OTHER; note = LNA thymine
  modified_base 17: mod_base = OTHER; note = LNA cytosine
ccgtccaaccccgcgtc

SEQ ID NO: 48 - DNA, 18 bp, synthetic construct
  source 1..18: mol_type = other DNA
  misc_feature 1..18: note = phosphothionate-modified backbone between every residue
  modified_base 1: mod_base = OTHER; note = LNA cytosine
  modified_base 2: mod_base = OTHER; note = LNA cytosine
  modified_base 3: mod_base = OTHER; note = LNA thymine
  modified_base 16: mod_base = OTHER; note = LNA guanine
  modified_base 17: mod_base = OTHER; note = LNA cytosine
  modified_base 18: mod_base = OTHER; note = LNA adenine
cctaaagctcctccagca

SEQ ID NO: 49 - DNA, 18 bp, synthetic construct
  source 1..18: mol_type = other DNA
  misc_feature 1..18: note = phosphothionate-modified backbone between every residue
  modified_base 1: mod_base = OTHER; note = LNA guanine
  modified_base 2: mod_base = OTHER; note = LNA cytosine
  modified_base 3: mod_base = OTHER; note = LNA guanine
  modified_base 16: mod_base = OTHER; note = LNA cytosine
  modified_base 17: mod_base = OTHER; note = LNA cytosine
  modified_base 18: mod_base = OTHER; note = LNA guanine
gcgaggcggcctcttccg

SEQ ID NO: 50 - DNA, 18 bp, synthetic construct
  source 1..18: mol_type = other DNA
  misc_feature 1..18: note = phosphothionate-modified backbone between every residue
  modified_base 1: mod_base = OTHER; note = LNA guanine
  modified_base 2: mod_base = OTHER; note = LNA cytosine
  modified_base 3: mod_base = OTHER; note = LNA cytosine
  modified_base 16: mod_base = OTHER; note = LNA guanine
  modified_base 17: mod_base = OTHER; note = LNA guanine
  modified_base 18: mod_base = OTHER; note = LNA guanine
gcctccagctcccccggg

SEQ ID NO: 51 - DNA, 17 bp, synthetic construct
  source 1..17: mol_type = other DNA
  misc_feature 1..17: note = phosphothionate-modified backbone between every residue
  modified_base 1: mod_base = OTHER; note = LNA guanine
  modified_base 2: mod_base = OTHER; note = LNA guanine
  modified_base 3: mod_base = OTHER; note = LNA thymine
  modified_base 15: mod_base = OTHER; note = LNA cytosine
  modified_base 16: mod_base = OTHER; note = LNA adenine
  modified_base 17: mod_base = OTHER; note = LNA thymine
ggtgtcgggagggccat

SEQ ID NO: 52 - DNA, 18 bp, synthetic construct
  source 1..18: mol_type = other DNA
  misc_feature 1..18: note = phosphothionate-modified backbone between every residue
  modified_base 1: mod_base = OTHER; note = LNA cytosine
  modified_base 2: mod_base = OTHER; note = LNA thymine
  modified_base 3: mod_base = OTHER; note = LNA guanine
  modified_base 16: mod_base = OTHER; note = LNA thymine
  modified_base 17: mod_base = OTHER; note = LNA cytosine
  modified_base 18: mod_base = OTHER; note = LNA thymine
ctgggccagccgttctct

SEQ ID NO: 53 - DNA, 18 bp, synthetic construct
  source 1..18: mol_type = other DNA
  misc_feature 1..18: note = phosphothionate-modified backbone between every residue
  modified_base 1: mod_base = OTHER; note = LNA guanine
  modified_base 2: mod_base = OTHER; note = LNA guanine
  modified_base 3: mod_base = OTHER; note = LNA guanine
  modified_base 16: mod_base = OTHER; note = LNA thymine
  modified_base 17: mod_base = OTHER; note = LNA guanine
  modified_base 18: mod_base = OTHER; note = LNA guanine
gggccagccgttctctgg

SEQ ID NO: 54 - DNA, 18 bp, synthetic construct
  source 1..18: mol_type = other DNA
  misc_feature 1..18: note = phosphothionate-modified backbone between every residue
  modified_base 1: mod_base = OTHER; note = LNA cytosine
  modified_base 2: mod_base = OTHER; note = LNA adenine
  modified_base 15: mod_base = OTHER; note = LNA thymine
  modified_base 17: mod_base = OTHER; note = LNA cytosine
  modified_base 18: mod_base = OTHER; note = LNA thymine
caatttcaggctttttct

SEQ ID NO: 55 - DNA, 18 bp, synthetic construct
  source 1..18: mol_type = other DNA
  misc_feature 1..18: note = phosphothionate-modified backbone between every residue
  modified_base 1: mod_base = OTHER; note = LNA thymine
  modified_base 2: mod_base = OTHER; note = LNA guanine
  modified_base 3: mod_base = OTHER; note = LNA cytosine
  modified_base 16: mod_base = OTHER; note = LNA thymine
  modified_base 17: mod_base = OTHER; note = LNA thymine
  modified_base 18: mod_base = OTHER; note = LNA guanine
tgcctacagaaggctttg

SEQ ID NO: 56 - DNA, 18 bp, synthetic construct
  source 1..18: mol_type = other DNA
  misc_feature 1..18: note = phosphothionate-modified backbone between every residue
  modified_base 1: mod_base = OTHER; note = LNA adenine
  modified_base 2: mod_base = OTHER; note = LNA thymine
  modified_base 15: mod_base = OTHER; note = LNA cytosine
  modified_base 17: mod_base = OTHER; note = LNA cytosine
  modified_base 18: mod_base = OTHER; note = LNA adenine
atctctgcactcatcaca

SEQ ID NO: 57 - DNA, 18 bp, synthetic construct
  source 1..18: mol_type = other DNA
  misc_feature 1..18: note = phosphothionate-modified backbone between every residue
  modified_base 1: mod_base = OTHER; note = LNA cytosine
  modified_base 2: mod_base = OTHER; note = LNA thymine
  modified_base 15: mod_base = OTHER; note = LNA thymine
  modified_base 17: mod_base = OTHER; note = LNA thymine
  modified_base 18: mod_base = OTHER; note = LNA guanine
ctgatcaccgaagttctg

SEQ ID NO: 58 - DNA, 18 bp, synthetic construct
  source 1..18: mol_type = other DNA
  misc_feature 1..18: note = phosphothionate-modified backbone between every residue
  modified_base 1: mod_base = OTHER; note = LNA adenine
  modified_base 2: mod_base = OTHER; note = LNA thymine
  modified_base 15: mod_base = OTHER; note = LNA guanine
  modified_base 17: mod_base = OTHER; note = LNA guanine
  modified_base 18: mod_base = OTHER; note = LNA guanine
ataggatccacagggagg

SEQ ID NO: 59 - DNA, 18 bp, synthetic construct
  source 1..18: mol_type = other DNA
  misc_feature 1..18: note = phosphothionate-modified backbone between every residue
  modified_base 1: mod_base = OTHER; note = LNA cytosine
  modified_base 2: mod_base = OTHER; note = LNA cytosine
  modified_base 15: mod_base = OTHER; note = LNA thymine
  modified_base 17: mod_base = OTHER; note = LNA thymine
  modified_base 18: mod_base = OTHER; note = LNA adenine
ccaggagatgtaactcta

SEQ ID NO: 60 - DNA, 18 bp, synthetic construct
  source 1..18: mol_type = other DNA
  misc_feature 1..18: note = phosphothionate-modified backbone between every residue
  modified_base 1: mod_base = OTHER; note = LNA guanine
  modified_base 2: mod_base = OTHER; note = LNA adenine
  modified_base 3: mod_base = OTHER; note = LNA adenine
  modified_base 16: mod_base = OTHER; note = LNA guanine
  modified_base 17: mod_base = OTHER; note = LNA cytosine
  modified_base 18: mod_base = OTHER; note = LNA cytosine
gaaagagaggccaccgcc

SEQ ID NO: 61 - DNA, 18 bp, synthetic construct
  source 1..18: mol_type = other DNA
  misc_feature 1..18: note = phosphothionate-modified backbone between every residue
  modified_base 1: mod_base = OTHER; note = LNA guanine
  modified_base 2: mod_base = OTHER; note = LNA thymine
  modified_base 3: mod_base = OTHER; note = LNA adenine
  modified_base 16: mod_base = OTHER; note = LNA thymine
  modified_base 17: mod_base = OTHER; note = LNA thymine
  modified_base 18: mod_base = OTHER; note = LNA cytosine
gtagccagccaggtgttc

SEQ ID NO: 62 - DNA, 18 bp, synthetic construct
  source 1..18: mol_type = other DNA
  misc_feature 1..18: note = phosphothionate-modified backbone between every residue
  modified_base 1: mod_base = OTHER; note = LNA guanine
  modified_base 2: mod_base = OTHER; note = LNA cytosine
  modified_base 3: mod_base = OTHER; note = LNA cytosine
  modified_base 16: mod_base = OTHER; note = LNA guanine
  modified_base 17: mod_base = OTHER; note = LNA cytosine
  modified_base 18: mod_base = OTHER; note = LNA cytosine
gcccctccgtagccagcc

SEQ ID NO: 63 - DNA, 18 bp, synthetic construct
  source 1..18: mol_type = other DNA
  misc_feature 1..18: note = phosphothionate-modified backbone between every residue
  modified_base 1: mod_base = OTHER; note = LNA thymine
  modified_base 2: mod_base = OTHER; note = LNA guanine
  modified_base 3: mod_base = OTHER; note = LNA cytosine
  modified_base 16: mod_base = OTHER; note = LNA thymine
  modified_base 17: mod_base = OTHER; note = LNA cytosine
  modified_base 18: mod_base = OTHER; note = LNA guanine
tgctgtccgagggtgtcg

SEQ ID NO: 64 - DNA, 18 bp, synthetic construct
  source 1..18: mol_type = other DNA
  misc_feature 1..18: note = phosphothionate-modified backbone between every residue
  modified_base 1: mod_base = OTHER; note = LNA adenine
  modified_base 2: mod_base = OTHER; note = LNA guanine
  modified_base 3: mod_base = OTHER; note = LNA guanine
  modified_base 16: mod_base = OTHER; note = LNA guanine
  modified_base 17: mod_base = OTHER; note = LNA adenine
  modified_base 18: mod_base = OTHER; note = LNA guanine
aggggtgcttccagcgag

SEQ ID NO: 65 - DNA, 18 bp, synthetic construct
  source 1..18: mol_type = other DNA
  misc_feature 1..18: note = phosphothionate-modified backbone between every residue
  modified_base 1: mod_base = OTHER; note = LNA thymine
  modified_base 2: mod_base = OTHER; note = LNA thymine
  modified_base 3: mod_base = OTHER; note = LNA cytosine
  modified_base 16: mod_base = OTHER; note = LNA guanine
  modified_base 17: mod_base = OTHER; note = LNA guanine
  modified_base 18: mod_base = OTHER; note = LNA guanine
ttcttcctcgctgagggg

SEQ ID NO: 66 - DNA, 18 bp, synthetic construct
  source 1..18: mol_type = other DNA
  misc_feature 1..18: note = phosphothionate-modified backbone between every residue
  modified_base 1: mod_base = OTHER; note = LNA cytosine
  modified_base 2: mod_base = OTHER; note = LNA guanine
  modified_base 3: mod_base = OTHER; note = LNA guanine
  modified_base 16: mod_base = OTHER; note = LNA cytosine
  modified_base 17: mod_base = OTHER; note = LNA thymine
  modified_base 18: mod_base = OTHER; note = LNA guanine
cggtattcttcctcgctg

SEQ ID NO: 67 - DNA, 18 bp, synthetic construct
  source 1..18: mol_type = other DNA
  misc_feature 1..18: note = phosphothionate-modified backbone between every residue
  modified_base 1: mod_base = OTHER; note = LNA thymine
  modified_base 2: mod_base = OTHER; note = LNA cytosine
  modified_base 3: mod_base = OTHER; note = LNA cytosine
  modified_base 16: mod_base = OTHER; note = LNA cytosine
  modified_base 17: mod_base = OTHER; note = LNA guanine
  modified_base 18: mod_base = OTHER; note = LNA guanine
tcctccagcagagcccgg

SEQ ID NO: 68 - DNA, 18 bp, synthetic construct
  source 1..18: mol_type = other DNA
  misc_feature 1..18: note = phosphothionate-modified backbone between every residue
  modified_base 1: mod_base = OTHER; note = LNA cytosine
  modified_base 2: mod_base = OTHER; note = LNA cytosine
  modified_base 3: mod_base = OTHER; note = LNA thymine
  modified_base 16: mod_base = OTHER; note = LNA guanine
  modified_base 17: mod_base = OTHER; note = LNA guanine
  modified_base 18: mod_base = OTHER; note = LNA adenine
cctgggccggctctggga

SEQ ID NO: 69 - DNA, 18 bp, synthetic construct
  source 1..18: mol_type = other DNA
  misc_feature 1..18: note = phosphothionate-modified backbone between every residue
  modified_base 1: mod_base = OTHER; note = LNA thymine
  modified_base 2: mod_base = OTHER; note = LNA guanine
  modified_base 3: mod_base = OTHER; note = LNA cytosine
  modified_base 16: mod_base = OTHER; note = LNA cytosine
  modified_base 17: mod_base = OTHER; note = LNA guanine
  modified_base 18: mod_base = OTHER; note = LNA guanine
tgctggtacctgggccgg

SEQ ID NO: 70 - DNA, 18 bp, synthetic construct
  source 1..18: mol_type = other DNA
  misc_feature 1..18: note = phosphothionate-modified backbone between every residue
  modified_base 1: mod_base = OTHER; note = LNA thymine
  modified_base 2: mod_base = OTHER; note = LNA cytosine
  modified_base 3: mod_base = OTHER; note = LNA thymine
  modified_base 16: mod_base = OTHER; note = LNA guanine
  modified_base 17: mod_base = OTHER; note = LNA guanine
  modified_base 18: mod_base = OTHER; note = LNA guanine
tctataggatccacaggg

(19) United States
(12) Patent Application Publication
(10) Pub. No.: US 2025/0025569 A1
(43) Pub. Date: Jan. 23, 2025

(54) TISSUE-SPECIFIC IMAGING AND THERAPEUTIC AGENTS TARGETING PROTEINS EXPRESSED ON MUSCLE CELL SURFACE

(71) Applicant: MIRECULE, INC., Gaithersburg, MD (US)

(72) Inventors: Tishan WILLIAMS, Gaithersburg, MD (US); Anthony SALEH, Gaithersburg, MD (US); Christian KINNEY, Gaithersburg, MD (US)

(21) Appl. No.: 18/762,429

(22) Filed: Jul. 2, 2024

Related U.S. Application Data
(63) Continuation of application No. PCT/US2023/060253, filed on Jan. 6, 2023.
(60) Provisional application No. 63/297,245, filed on Jan. 7, 2022.

(51) Int. Cl. A61K 47/68; A61K 49/00; C12N 15/113

(52) U.S. Cl. CPC A61K 47/6807; A61K 47/6849; A61K 49/0058; C12N 15/113; A61K 2123/00; C12N 2310/11

(57) ABSTRACT

Disclosed herein are muscle-targeted agents for tissue-specific delivery of therapeutic and diagnostic agents. Also disclosed herein are methods of delivering an agent in a tissue-specific manner, particularly muscle tissue, by targeting a protein expressed on the cell surface of muscle tissue. The methods can be used for detecting, imaging and/or treating pathologies, as well as for diagnostics.

### CROSS-REFERENCE TO RELATED APPLICATIONS

This application is a continuation of International Patent Application No. PCT/US2023/060253, filed Jan. 6, 2023, which claims priority to U.S. Provisional Patent Application Ser. No. 63/297,245, filed Jan. 7, 2022, the entire disclosures of which are hereby incorporated herein by reference.

### STATEMENT OF GOVERNMENT SUPPORT

This invention was made with government support under R44 NS119147 awarded by the National Institutes of Health. The government has certain rights in the invention.

### SEQUENCE LISTING

The instant application contains a Sequence Listing which has been submitted electronically in XML format and is hereby incorporated by reference in its entirety. Said XML file, created on Jul. 2, 2024, is named 754920_SA9-372PCCON_ST26.xml and is 294,257 bytes in size.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BACKGROUND

There are currently no cures and limited treatment options for a range of muscle involved disorders. For example, current treatments for muscular dystrophies include Eteplirsen, Viltolarsen, Golodirsen, and Casimersen, which are all non-targeted anti-sense oligonucleotides. Eteplirsen is administered intravenously weekly at 30 mg/kg. Two-thirds of the dose is lost within 24 hours of administration due to renal clearance. Viltolarsen is also administered intravenously at 80 mg/kg. The recommended dosage of Casimersen is 30 mg/kg administered once weekly as a 35- to 60-minute intravenous infusion via an in-line 0.2 micron filter. The recommended dosage of Golodirsen is 30 mg/kg administered once weekly as a 35- to 60-minute intravenous infusion via an in-line 0.2 micron filter.

It would be beneficial to be able to target therapeutics selectively to muscle tissue using therapeutic targeting agents. One example therapeutic targeting agent is antibody drug conjugates (ADCs). ADCs have been proven useful in oncology and other disorders to improve delivery to the target tissue, increasing the potency of the conjugated drug and the therapeutic index enabling safe use of many compounds.

Of particular interest is targeted delivery of oligonucleotides to muscle tissue via conjugation to antibodies against muscle-specific or muscle-enriched surface antigens. This targeted delivery should create better dose efficacy to toxicity ratio, increase therapeutic half-life, reduce loss due to renal clearance, and minimize off-target effects. This emerging strategy would greatly expand the therapeutic landscape to treat a wide range of muscle-associated diseases such as Duchenne Muscular Dystrophy, Facioscapulohumeral Muscular Dystrophy (FSHD), Diabetes or cardiomyopathy. This field has been held back by the dearth of muscle specific receptors that are stably expressed in disease tissue that would enable antibody targeted delivery. Further, many muscle related diseases are rare disorders with small patient populations. Since it costs tens to hundreds of millions of US dollars to develop an antibody therapeutic, development of treatments for these rare disorders would be greatly enhanced by the availability of one or more muscle-specific antibodies that could be used for ADCs across multiple disorders. Thus, there is a need to develop muscle-specific therapeutic targeting agents directed against muscle specific receptors that are stably expressed in disease tissue.

### SUMMARY

Disclosed herein are methods of delivering an agent to muscle tissue in vivo in a tissue-specific manner which can comprise contacting the surface of muscle cells with an agent that specifically binds a targeted protein expressed on a cell surface of the muscle tissue. In some embodiments, a targeted protein can be enriched in muscle tissue relative to other tissues. In some embodiments, a targeted protein can have stable or increased expression in diseased tissue relative to normal tissue. In some embodiments, a targeted protein can be internalized and recycled within from about 2 minutes to about 12 hours, from about 2 minutes to about 10 hours, from about 2 minutes to about 8 hours, from about 2 minutes to about 6 hours, from about 2 minutes to about 4 hours, from about 2 minutes to about 2 hours, from about 2 minutes to about 60 minutes, from about 2 minutes to about 40 minutes, from about 2 minutes to about 20 minutes, from about 2 minutes to about 10 minutes, from about 10 minutes to about 12 hours, from about 20 minutes to about 12 hours, from about 40 minutes to about 12 hours, from about 60 minutes to about 12 hours, from about 2 hours to about 12 hours, from about 4 hours to about 12 hours, from about 6 hours to about 12 hours, from about 8 hours to about 12 hours, from about 10 hours to about 12 hours, or from about 11 hours to about 12 hours.

In some embodiments, a targeted protein can be selected from the group consisting of: KLHL41, LMOD2, ENO3, FABP3, CHRNA1, SEMA6C, XIRP2, XIRP1, CAVIN4, CFL2, SVIL, MUSK, ART1, CACNA1S, CDH15, CLCN1, MYMX, and ACTA1. In some embodiments, an agent is a specific binding agent of the targeted protein. In some embodiments, a specific binding agent can be a soluble receptor or a soluble ligand. In some embodiments, a soluble receptor can comprise the extracellular domain of a receptor. In some embodiments, a soluble receptor can be a Fc fusion protein.

In some embodiments, an agent can be an antibody or an antigen-binding fragment thereof. In some embodiments, an antibody or antigen-binding fragment thereof can be selected from the group consisting of a monoclonal antibody, a bispecific antibody, a Fab, a Fab-Fc, a Fv, a single chain Fv (scFv), a diabody, a minibody, a VNAR, and an immunoglobulin single variable domain (ISV) such as an NANOBODY® molecule.

Also disclosed herein are methods of treating a pathology in an individual which can comprise administering to an individual a therapeutic targeting agent that specifically binds a targeted protein expressed on muscle tissue cell surface. In some embodiments, a targeted protein can be enriched in muscle tissue relative to other tissues. In some embodiments, a targeted protein can have stable or increased expression in diseased tissue relative to normal tissue In some embodiments, a targeted protein can be internalized and recycled within from about 2 minutes to about 12 hours, from about 2 minutes to about 10 hours, from about 2 minutes to about 8 hours, from about 2 minutes to about 6 hours, from about 2 minutes to about 4 hours, from about 2 minutes to about 2 hours, from about 2 minutes to about 60 minutes, from about 2 minutes to about 40 minutes, from about 2 minutes to about 20 minutes, from about 2 minutes to about 10 minutes, from about 10 minutes to about 12 hours, from about 20 minutes to about 12 hours, from about 40 minutes to about 12 hours, from about 60 minutes to about 12 hours, from about 2 hours to about 12 hours, from about 4 hours to about 12 hours, from about 6 hours to about 12 hours, from about 8 hours to about 12 hours, from about 10 hours to about 12 hours, or from about 11 hours to about 12 hours.

In some embodiments, a therapeutic targeting agent can be an agent that comprises an active agent component and a targeting agent component. In some embodiments, an active agent component can be selected from the group consisting of: a radionuclide; a chemotherapeutic agent; an immune stimulatory agent; an anti-neoplastic agent; an anti-inflammatory agent; a pro-inflammatory agent; a pro-apoptotic agent; a pro-coagulant; a toxin; an antibiotic; a hormone; an enzyme; a protein; a carrier protein; a lytic agent; a small molecule; aptamers; cells, vaccine-induced or other immune cells; nanoparticles; transferrins; immunoglobulins; multivalent antibodies; lipids; lipoproteins; liposomes; an altered natural ligand; a gene or nucleic acid; an oligonucleotide; RNA; siRNA; an ncRNA mimic; a short-harpin RNA (shRNA); a dicer-dependent siRNA (di-siRNA); an antisense oligonucleotide (ASO); a gapmer; a mixmer; a double-stranded RNA (dsRNA); a single stranded RNAi (ssRNAi); a DNA-directed RNA interference (ddRNAi); an RNA activating oligonucleotide (RNAa); an aptamer; an exon skipping oligonucleotide; a miRNA; a miRNA mimic; an mRNA; a guide RNA; a viral or non-viral gene delivery vector; a prodrug; and a promolecule. In some embodiments, a targeting agent component can specifically bind to the targeted protein.

In some embodiments, a targeting agent component can comprise a specific binding agent of the targeted protein. In some embodiments, a specific binding agent can be a soluble receptor or a soluble ligand. In some embodiments, a soluble receptor can comprise the extracellular domain of a receptor. In some embodiments, a soluble receptor can be a Fc fusion protein. In some embodiments, a targeting agent component can comprise an antibody or an antigen-binding fragment thereof. In some embodiments, an antibody or antigen-binding fragment thereof can be selected from the group consisting of a monoclonal antibody, a bispecific antibody, a Fab, a Fab-Fc, a Fv, a single chain Fv (scFv), a diabody, a minibody, a VNAR, and a immunoglobulin single variable domain (ISV) such as an NANOBODY® molecule. In some embodiments, an active agent component can an oligonucleotide. In some embodiments, a targeting agent component can be an antibody or an antigen-binding fragment thereof. In some embodiments, an active agent component can be conjugated to the targeting agent component. In some embodiments, an oligonucleotide can target a disease gene expressed in muscle tissue. In some embodiments, a targeted protein can be selected from the group consisting of: KLHL41, LMOD2, ENO3, FABP3, CHRNA1, SEMA6C, XIRP2, XIRP1, CAVIN4, CFL2, SVIL, MUSK, ART1, CACNA1S, CDH15, CLCN1, MYMX, and ACTA1.

In some embodiments, a targeted protein is KLHL41. In some embodiments, a targeted protein is LMOD2. In some embodiments, a targeted protein is ENO3. In some embodiments, a targeted protein is FABP3. In some embodiments, a targeted protein is CHRNA1. In some embodiments, a targeted protein is SEMA6C. In some embodiments, a targeted protein is XIRP2. In some embodiments, a targeted protein is XIRP1. In some embodiments, a targeted protein is CAVIN4. In some embodiments, a targeted protein is CFL2. In some embodiments, a targeted protein is SVIL. In some embodiments, a targeted protein is MUSK. In some embodiments, a targeted protein is ART1. In some embodiments, a targeted protein is CACNA1S. In some embodiments, a targeted protein is CDH15. In some embodiments, a targeted protein is CLCN1. In some embodiments, a targeted protein is CLCN1. In some embodiments, a targeted protein is MYMX. In some embodiments, a targeted protein is ACTA1.

Also disclosed herein are methods of delivering an imaging agent to muscle tissue in a tissue-specific manner which can comprise contacting the surface of muscle cells with an imaging agent that can comprise an imaging agent component and a targeting agent component. In some embodiments, a targeting agent component can specifically bind to a targeted protein expressed on the cell surface of the tissue. In some embodiments, a targeted protein can be enriched in muscle tissue relative to other tissues. In some embodiments, a targeted protein can have stable or increased expression in diseased tissue relative to normal tissue. In some embodiments, a targeted protein can be internalized and recycled within from about 2 minutes to about 12 hours, from about 2 minutes to about 10 hours, from about 2 minutes to about 8 hours, from about 2 minutes to about 6 hours, from about 2 minutes to about 4 hours, from about 2 minutes to about 2 hours, from about 2 minutes to about 60 minutes, from about 2 minutes to about 40 minutes, from about 2 minutes to about 20 minutes, from about 2 minutes to about 10 minutes, from about 10 minutes to about 12 hours, from about 20 minutes to about 12 hours, from about 40 minutes to about 12 hours, from about 60 minutes to about 12 hours, from about 2 hours to about 12 hours, from about 4 hours to about 12 hours, from about 6 hours to about 12 hours, from about 8 hours to about 12 hours, from about 10 hours to about 12 hours, or from about 11 hours to about 12 hours.

In some embodiments, a targeted protein can be selected from the group consisting of: KLHL41, LMOD2, ENO3, FABP3, CHRNA1, SEMA6C, XIRP2, XIRP1, CAVIN4, CFL2, SVIL, MUSK, ART1, CACNA1S, CDH15, CLCN1, MYMX, and ACTA1. In some embodiments, a targeting agent component can be a specific binding agent of the targeted protein. In some embodiments, a specific binding agent can be a soluble receptor or a soluble ligand. In some embodiments, a soluble receptor can comprise the extracellular domain of a receptor. In some embodiments, a soluble receptor can be a Fc fusion protein.

In some embodiments, a targeting agent component can be an antibody or an antigen-binding fragment thereof. In some embodiments, an antibody or antigen-binding fragment thereof can be selected from the group consisting of a monoclonal antibody, a bispecific antibody, a Fab, a Fab-Fc, a Fv, a single chain Fv (scFv), a diabody, a minibody, a VNAR, and a immunoglobulin single variable domain (ISV) such as an NANOBODY® molecule. In some embodiments, an imaging agent component can be selected from the group consisting of: a radioactive agent, radioisotope or radiopharmaceutical; a contrast agent; a magnetic agent or a paramagnetic agent; liposomes; ultrasound agents; a gene vector or virus inducing a detecting agent; an enzyme; a prosthetic group; a fluorescent material; a luminescent material; and a bioluminescent material.

Also disclosed herein are methods of delivering an imaging agent in a tissue-specific manner to a tissue sample which can comprise contacting the tissue sample with an imaging agent that can comprise an imaging agent component and a targeting agent component. In some embodiments, a targeting agent component can specifically bind to a targeted protein expressed on a muscle cell surface of the tissue. In some embodiments, a targeted protein can be enriched in muscle tissue relative to other tissues. In some embodiments, a targeted protein can have stable or increased expression in diseased tissue relative to normal tissue. In some embodiments, a targeted protein can be internalized and recycled within from about 2 minutes to about 12 hours, from about 2 minutes to about 10 hours, from about 2 minutes to about 8 hours, from about 2 minutes to about 6 hours, from about 2 minutes to about 4 hours, from about 2 minutes to about 2 hours, from about 2 minutes to about 60 minutes, from about 2 minutes to about 40 minutes, from about 2 minutes to about 20 minutes, from about 2 minutes to about 10 minutes, from about 10 minutes to about 12 hours, from about 20 minutes to about 12 hours, from about 40 minutes to about 12 hours, from about 60 minutes to about 12 hours, from about 2 hours to about 12 hours, from about 4 hours to about 12 hours, from about 6 hours to about 12 hours, from about 8 hours to about 12 hours, from about 10 hours to about 12 hours, or from about 11 hours to about 12 hours.

In some embodiments, a targeted protein can be selected from the group consisting of: KLHL41, LMOD2, ENO3, FABP3, CHRNA1, SEMA6C, XIRP2, XIRP1, CAVIN4, CFL2, SVIL, MUSK, ART1, CACNA1S, CDH15, CLCN1, MYMX, and ACTA1. In some embodiments, a targeting agent component can be a specific binding agent of the targeted protein. In some embodiments, a specific binding agent can be a soluble receptor or a soluble ligand. In some embodiments, a soluble receptor can comprise the extracellular domain of a receptor. In some embodiments, a soluble receptor can be a Fc fusion protein.

In some embodiments, a targeting agent component can be an antibody or an antigen-binding fragment thereof. In some embodiments, an antibody or antigen-binding fragment thereof is selected from the group consisting of a monoclonal antibody, a bispecific antibody, a Fab, a Fab-Fc, a Fv, a single chain Fv (scFv), a diabody, a minibody, a VNAR, and a immunoglobulin single variable domain (ISV) such as an NANOBODY® molecule. In some embodiments, an imaging agent component is selected from the group consisting of: a radioactive agent, radioisotope or radiopharmaceutical; a contrast agent; a magnetic agent or a paramagnetic agent; liposomes; ultrasound agents; a gene vector or virus inducing a detecting agent; an enzyme; a prosthetic group; a fluorescent material; a luminescent material; and a bioluminescent material.

Also disclosed herein are methods of performing physical imaging of muscle tissue of an individual which can comprise administering to the individual an imaging agent which can comprise a targeting agent component and an imaging agent component. In some embodiments, a targeting agent component can specifically bind to a targeted protein expressed on the cell surface of the muscle tissue In some embodiments, a targeted protein can be enriched in muscle tissue relative to other tissues In some embodiments, a targeted protein can have stable or increased expression in diseased tissue relative to normal tissue. In some embodiments, a targeted protein can be internalized and recycled within from about 2 minutes to about 12 hours, from about 2 minutes to about 10 hours, from about 2 minutes to about 8 hours, from about 2 minutes to about 6 hours, from about 2 minutes to about 4 hours, from about 2 minutes to about 2 hours, from about 2 minutes to about 60 minutes, from about 2 minutes to about 40 minutes, from about 2 minutes to about 20 minutes, from about 2 minutes to about 10 minutes, from about 10 minutes to about 12 hours, from about 20 minutes to about 12 hours, from about 40 minutes to about 12 hours, from about 60 minutes to about 12 hours, from about 2 hours to about 12 hours, from about 4 hours to about 12 hours, from about 6 hours to about 12 hours, from about 8 hours to about 12 hours, from about 10 hours to about 12 hours, or from about 11 hours to about 12 hours.

In some embodiments, a targeted protein can be selected from the group consisting of: KLHL41, LMOD2, ENO3, FABP3, CHRNA1, SEMA6C, XIRP2, XIRP1, CAVIN4, CFL2, SVIL, MUSK, ART1, CACNA1S, CDH15, CLCN1, MYMX, and ACTA1. In some embodiments, an imaging agent component is selected from the group consisting of: a radioactive agent, radioisotope or radiopharmaceutical; a contrast agent; a magnetic agent or a paramagnetic agent; liposomes; ultrasound agents; a gene vector or virus inducing a detecting agent; an enzyme; a prosthetic group; a fluorescent material; a luminescent material; and a bioluminescent material.

Also disclosed herein are methods of assessing an individual for the presence or absence of a muscle tissue pathology which can comprise administering to the individual an imaging agent that can comprise an imaging agent component and a targeting agent component and assessing the individual for the presence or absence of a concentration of the imaging agent. In some embodiments, a targeting agent component can specifically bind to a targeted protein expressed on the cell surface of the muscle tissue. In some embodiments, the presence or absence of a concentration of the imaging agent can be indicative of the presence of the pathology. In some embodiments, a targeting agent component can be a specific binding agent of the targeted protein. In some embodiments, a specific binding agent can be a soluble receptor or a soluble ligand. In some embodiments, a soluble receptor can comprise the extracellular domain of a receptor. In some embodiments, a soluble receptor can be a Fc fusion protein.

In some embodiments, a targeting agent component can be an antibody or an antigen-binding fragment thereof. In some embodiments, an antibody or antigen-binding fragment thereof can be selected from the group consisting of a monoclonal antibody, a bispecific antibody, a Fab, a Fab-Fc, a Fv, a single chain Fv (scFv), a diabody, a minibody, a VNAR, and a immunoglobulin single variable domain (ISV) such as an NANOBODY® molecule. In some embodiments, a targeted protein can be selected from the group consisting of: KLHL41, LMOD2, ENO3, FABP3, CHRNA1, SEMA6C, XIRP2, XIRP1, CAVIN4, CFL2, SVIL, MUSK, ART1, CACNA1S, CDH15, CLCN1, MYMX, and ACTA1. In some embodiments, an imaging agent component can be selected from the group consisting of: a radioactive agent, radioisotope or radiopharmaceutical; a contrast agent; a magnetic agent or a paramagnetic agent; liposomes; ultrasound agents; a gene vector or virus inducing a detecting agent; an enzyme; a prosthetic group; a fluorescent material; a luminescent material; and a bioluminescent material.

Also disclosed are methods of assessing response of muscle tissue from an individual to treatment with a therapeutic targeting agent which can comprise assessing the level of the targeted protein in a sample from the individual before treatment with the therapeutic targeting agent; assessing the level of the targeted protein in a sample from the individual during or after treatment with the therapeutic targeting agent; and then comparing the level before treatment with the level during or after treatment. In some embodiments, a therapeutic targeting agent can specifically bind a targeted protein expressed on the cell surface of the muscle tissue. In some embodiments, a level of the targeted protein during or after treatment that is significantly lower than the level of the targeted protein before treatment can be indicative of efficacy of treatment with the therapeutic targeting agent. In some embodiments, a therapeutic targeting agent can specifically bind a targeted protein expressed on the cell surface of the muscle tissue. In some embodiments, a level of the targeted protein during or after treatment that is lower than the level of the targeted protein before treatment can be indicative of efficacy of treatment with the therapeutic targeting agent. In some embodiments, a targeted protein can be enriched in muscle tissue relative to other tissues. In some embodiments, a targeted protein can have stable or increased expression in diseased tissue relative to normal tissue. In some embodiments, a targeted protein can internalized and recycled within from about 2 minutes to about 12 hours, from about 2 minutes to about 10 hours, from about 2 minutes to about 8 hours, from about 2 minutes to about 6 hours, from about 2 minutes to about 4 hours, from about 2 minutes to about 2 hours, from about 2 minutes to about 60 minutes, from about 2 minutes to about 40 minutes, from about 2 minutes to about 20 minutes, from about 2 minutes to about 10 minutes, from about 10 minutes to about 12 hours, from about 20 minutes to about 12 hours, from about 40 minutes to about 12 hours, from about 60 minutes to about 12 hours, from about 2 hours to about 12 hours, from about 4 hours to about 12 hours, from about 6 hours to about 12 hours, from about 8 hours to about 12 hours, from about 10 hours to about 12 hours, or from about 11 hours to about 12 hours.

In some embodiments, a targeting protein can be selected from the group consisting of: KLHL41, LMOD2, ENO3, FABP3, CHRNA1, SEMA6C, XIRP2, XIRP1, CAVIN4, CFL2, SVIL, MUSK, ART1, CACNA1S, CDH15, CLCN1, MYMX, and ACTA1.

### DESCRIPTION OF THE DRAWINGS

The novel features of exemplary embodiments are set forth with particularity in the appended claims. A better understanding of the features and advantages will be obtained by reference to the following detailed description that sets forth illustrative embodiments, in which the principles of exemplary embodiments are utilized, and the accompanying drawings of which:

FIG. 1 shows a schematic overview of our executed analysis strategy to identify muscle specific targets stably expressed across muscle related diseases suitable for targeting therapeutic agents.

FIG. 2 shows fluorescence microscopy images of immunofluorescence validation of muscle membrane receptor expression and accessibility to antibody binding in live differentiated human myotubes.

FIG. 3 shows fluorescence microscopy images of immunofluorescence validation of muscle membrane receptor expression and accessibility to antibody binding in live differentiated mouse myotubes.

FIGS. 4A and 4B show fluorescence microscopy images of immunofluorescence Low/No expression of selected receptors on non-muscle cells by immunofluorescence.

FIGS. 5A and 5B show fluorescence microscopy images of receptor-mediated antibody-conjugate internalization by immunofluorescence.

### DETAILED DESCRIPTION

### General

Practice of the methods, as well as preparation and use of the compositions disclosed herein employ, unless otherwise indicated, conventional techniques in molecular biology, biochemistry, chromatin structure and analysis, computational chemistry, cell culture, recombinant DNA and related fields as are within the skill of the art. These techniques are fully explained in the literature. See, for example, Sambrook et al. MOLECULAR CLONING: A LABORATORY MANUAL, Second edition, Cold Spring Harbor Laboratory Press, 1989 and Third edition, 2001; Ausubel et al., CURRENT PROTOCOLS IN MOLECULAR BIOLOGY, John Wiley & Sons, New York, 1987 and periodic updates; the series METHODS IN ENZYMOLOGY, Academic Press, San Diego; Wolffe, CHROMATIN STRUCTURE AND FUNCTION, Third edition, Academic Press, San Diego, 1998; METHODS IN ENZYMOLOGY, Vol. 304, “Chromatin” (P. M. Wassarman and A. P. Wolffe, eds.), Academic Press, San Diego, 1999; and METHODS IN MOLECULAR BIOLOGY, Vol. 119, “Chromatin Protocols” (P. B. Becker, ed.) Humana Press, Totowa, 1999.

### Definitions

The term “herein” means the entire application.

Unless otherwise defined herein, scientific and technical terms used in this application shall have the meanings that are commonly understood by those of ordinary skill in the art to which this specification belongs. Generally, nomenclature used in connection with the compounds, composition and methods described herein, are those well-known and commonly used in the art.

It should be understood that any of the embodiments described herein, including those described under different aspects of the disclosure and different parts of the specification (including embodiments described only in the Examples) can be combined with one or more other embodiments herein, unless explicitly disclaimed or improper. Combination of embodiments are not limited to those specific combinations claimed via the multiple dependent claims.

All of the above, and any other publications, patents and published patent applications referred to in this application are specifically incorporated by reference herein. In case of conflict, the present specification, including its specific definitions, will control.

Throughout this specification, the word “comprise” or variations such as “comprises” or “comprising” will be understood to imply the inclusion of a stated integer (or components) or group of integers (or components), but not the exclusion of any other integer (or components) or group of integers (or components).

Throughout the specification, where compositions are described as having, including, or comprising (or variations thereof), specific components, it is contemplated that compositions also may consist essentially of, or consist of, the recited components. Similarly, where methods or processes are described as having, including, or comprising specific process steps, the processes also may consist essentially of, or consist of, the recited processing steps. Further, it should be understood that the order of steps or order for performing certain actions is immaterial so long as the compositions and methods described herein remains operable. Moreover, two or more steps or actions can be conducted simultaneously.

The term “including” is used to mean “including but not limited to.” “Including” and “including but not limited to” are used interchangeably.

As used herein, “about” or “approximately” means within an acceptable error range for the particular value as determined by one of ordinary skill in the art, which will depend in part on how the value is measured or determined, i.e., the limitations of the measurement system. For example, “about” can mean plus or minus 10%, per the practice in the art. Alternatively, “about” can mean a range of plus or minus 20%, plus or minus 10%, plus or minus 5%, or plus or minus 1% of a given value. Alternatively, particularly with respect to biological systems or processes, the term can mean within an order of magnitude, within 5-fold, or within 2-fold, of a value. Where particular values are described in the application and claims, unless otherwise stated the term “about” meaning within an acceptable error range for the particular value should be assumed. Also, where ranges and/or subranges of values are provided, the ranges and/or subranges can include the endpoints of the ranges and/or subranges.

The term “substantially” as used herein can refer to a value approaching 100% of a given value. In some cases, the term can refer to an amount that can be at least about 90%, 91%, 92%, 93%, 94%, 95%, 96%, 97%, 98%, 99%, 99.9%, or 99.99% of a total amount. In some cases, the term can refer to an amount that can be about 100% of a total amount.

The use of the terms “a” and “an” and “the” and similar referents in the context of describing the elements (especially in the context of the following claims) are to be construed to cover both the singular and the plural, unless otherwise indicated herein or clearly contradicted by context.

The term “or” as used herein should be understood to mean “and/or,” unless the context clearly indicates otherwise.

Statistical significance is used to determine whether the null hypothesis should be rejected or retained. The null hypothesis is the default assumption that nothing happened or changed. I.e., a difference or deviation, e.g., a significant lower protein expression in one sample compared to another sample, is significant if the null hypothesis is rejected. For the null hypothesis to be rejected, an observed result has to be statistically significant, i.e. the observed p-value is less than the pre-specified significance level α. To determine whether a result is statistically significant, the person skilled in the art calculates a p-value, which is the probability of observing an effect of the same magnitude or more extreme given that the null hypothesis is true. The null hypothesis is rejected if the p-value is less than (or equal to) a predetermined level, α. α is also called the significance level, and is the probability of rejecting the null hypothesis given that it is true (a type I error). α may be set at 5%. α may be set at 1%. α may be set at 0.1%. α may be set at 0.01%.

Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the embodiments and does not pose a limitation on the scope of the claims unless otherwise stated. No language in the specification should be construed as indicating any non-claimed element as essential.

The terms “nucleic acid,” “polynucleotide,” and “oligonucleotide” are used interchangeably and refer to a deoxyribonucleotide or ribonucleotide polymer, in linear or circular conformation, and in either single- or double-stranded form. For the purposes of the present disclosure, these terms are not to be construed as limiting with respect to the length of a polymer. The terms can encompass known analogues of natural nucleotides, as well as nucleotides that are modified in the base, sugar and/or phosphate moieties (e.g., phosphorothioate backbones, 2′-deoxy-, 2′-O-methyl-, 2′-deoxy-2′-fluoro-modified nucleotides, and a terminal cap molecule at the 3′-end, the 5′-end, or both of the 3′- and 5′-ends). In general, an analogue of a particular nucleotide has the same base-pairing specificity; i.e., an analogue of A will base-pair with T. The terms also encompass polymers comprising one or more chemically modified nucleotides. Non-limiting examples of polynucleotides include small interfering RNAs (siRNAs), microRNAs (miRNAs), miRNA mimics, short hairpin RNA (shRNA), double-stranded RNA (dsRNA), transfer RNA (tRNA), ribosomal RNA (rRNA), heterogeneous nuclear RNA (hnRNA), antisense oligonucleotides (ASOs, including exon-skipping ASOs), messenger RNAs (mRNAs), complementary DNAs (cDNAs), plasmids and vectors, and guide RNAs (gRNAs).

An oligonucleotide can comprise a sugar modification. An oligonucleotide can comprise a plurality of sugar modifications. A sugar modification can comprise a glucose or derivative thereof. A sugar modification can comprise a ribose or deoxyribose. A sugar modification can comprise a monosaccharide, a disaccharide, a trisaccharide or any combination thereof.

The terms “polypeptide,” “peptide” and “protein” are used interchangeably to refer to a polymer of amino acid residues. The term also applies to amino acid polymers in which one or more amino acids are chemical analogues or modified derivatives of a corresponding naturally-occurring amino acids.

The term “residue,” as used herein, refers to a position in a protein and its associated amino acid identity.

The term “homology” can refer to a % identity of a sequence to a reference sequence. As a practical matter, whether any particular sequence can be at least 50%, 60%, 70%, 80%, 85%, 90%, 92%, 95%, 96%, 97%, 98% or 99% identical to any sequence described herein (which can correspond with a particular nucleic acid sequence described herein), such particular polypeptide sequence can be determined conventionally using known computer programs such the Bestfit program (Wisconsin Sequence Analysis Package, Version 8 for Unix, Genetics Computer Group, University Research Park, 575 Science Drive, Madison, Wis. 53711). When using Bestfit or any other sequence alignment program to determine whether a particular sequence is, for instance, 95% identical to a reference sequence, the parameters can be set such that the percentage of identity is calculated over the full length of the reference sequence and that gaps in homology of up to 5% of the total reference sequence are allowed.

For example, in a specific embodiment the identity between a reference sequence (query sequence, i.e., a sequence as described herein) and a subject sequence, also referred to as a global sequence alignment, can be determined using the FASTDB computer program based on the algorithm of Brutlag et al. (Comp. App. Biosci. 6:237-245 (1990)). In some cases, parameters for a particular embodiment in which identity is narrowly construed, used in a FASTDB amino acid alignment, can include: Scoring Scheme=PAM (Percent Accepted Mutations) 0, k-tuple=2, Mismatch Penalty=1, Joining Penalty=20, Randomization Group Length=0, Cutoff Score=1, Window Size=sequence length, Gap Penalty=5, Gap Size Penalty=0.05, Window Size=500 or the length of the subject sequence, whichever is shorter. According to this embodiment, if the subject sequence is shorter than the query sequence due to N- or C-terminal deletions, not because of internal deletions, a manual correction can be made to the results to take into consideration the fact that the FASTDB program does not account for N- and C-terminal truncations of the subject sequence when calculating global percent identity. For subject sequences truncated at the N- and C-termini, relative to the query sequence, the percent identity can be corrected by calculating the number of residues of the query sequence that are lateral to the N- and C-terminal of the subject sequence, which are not matched/aligned with a corresponding subject residue, as a percent of the total bases of the query sequence. A determination of whether a residue is matched/aligned can be determined by results of the FASTDB sequence alignment. This percentage can be then subtracted from the percent identity, calculated by the FASTDB program using the specified parameters, to arrive at a final percent identity score. This final percent identity score can be used for the purposes of this embodiment. In some cases, only residues to the N- and C-termini of the subject sequence, which are not matched/aligned with the query sequence, are considered for the purposes of manually adjusting the percent identity score. That is, only query residue positions outside the farthest N- and C-terminal residues of the subject sequence are considered for this manual correction. For example, a 90 residue subject sequence can be aligned with a 100 residue query sequence to determine percent identity. The deletion occurs at the N-terminus of the subject sequence and therefore, the FASTDB alignment does not show a matching/alignment of the first 10 residues at the N-terminus. The 10 unpaired residues represent 10% of the sequence (number of residues at the N- and C-termini not matched/total number of residues in the query sequence) so 10% is subtracted from the percent identity score calculated by the FASTDB program. If the remaining 90 residues were perfectly matched the final percent identity would be 90%. In another example, a 90 residue subject sequence is compared with a 100 residue query sequence. This time the deletions are internal deletions so there are no residues at the N- or C-termini of the subject sequence which are not matched/aligned with the query. In this case the percent identity calculated by FASTDB is not manually corrected. Once again, only residue positions outside the N- and C-terminal ends of the subject sequence, as displayed in the FASTDB alignment, which are not matched/aligned with the query sequence are manually corrected for.

The term “fragment,” as used herein, can be a portion of a sequence, a subset that can be shorter than a full-length sequence. A fragment can be a portion of a gene. A fragment can be a portion of a peptide or protein. A fragment can be a portion of an amino acid sequence. A fragment can be a portion of an oligonucleotide sequence. A fragment can be less than about: 20, 30, 40, 50 amino acids in length. A fragment can be less than about: 2, 5, 10, 20, 30, 40, 50 oligonucleotides in length.

As used herein, the terms “Fc,” “Fc region” or “Fc domain” are used interchangeably herein and refer to the polypeptide comprising the constant region of an antibody excluding, in some instances, the first constant region immunoglobulin domain (e.g., CH1) or a portion thereof, and in some cases, part of the hinge. Thus, an Fc can refer to the last two constant region immunoglobulin domains (e.g., CH2 and CH3) of IgA, IgD, and IgG, the last three constant region immunoglobulin domains of IgE and IgM, and the flexible hinge N-terminal to these domains. For IgA and IgM, Fc may include the J chain. For IgG, the Fc domain comprises immunoglobulin domains Cγ2 and Cγ3 (Cγ2 and Cγ3) and the lower hinge region between Cγ1 (Cγ1) and Cγ2 (Cγ2). In some embodiments, an Fc refers to a truncated CH1 domain, and CH2 and CH3 of an immunoglobulin. Although the boundaries of the Fc region may vary, the human IgG heavy chain Fc region is usually defined to include residues E216 or C226 or P230 to its carboxyl-terminus, wherein the numbering is according to the EU numbering. In some embodiments, the Fc domain is derived from a human IgG1 heavy chain Fc domain. In some embodiments, the Fc domain is derived from a human IgG2 heavy chain Fc domain. The “EU format as set forth in Edelman” or “EU numbering” or “EU index” refers to the residue numbering of the human Fc domain as described in Edelman G M et al. (Proc. Natl. Acad. USA (1969), 63, 78-85, hereby entirely incorporated by reference).

As used herein, the terms “Fc fusion protein” refers to a protein comprising an Fc region, generally linked (optionally through a linker moiety) to a different protein.

As used herein, the term “antibody” or “Ab” refers to an immunoglobulin molecule (e.g., complete antibodies, antibody fragment or modified antibodies) capable of recognizing and binding to a specific target or antigen, such as a carbohydrate, polynucleotide, lipid, polypeptide, etc., through at least one antigen recognition site, located in the variable region of the immunoglobulin molecule. As used herein, the term “antibody” can encompass any type of antibody, including but not limited to monoclonal antibodies, polyclonal antibodies, human antibodies, engineered antibodies (including humanized antibodies, fully human antibodies, chimeric antibodies, single-chain antibodies, artificially selected antibodies, CDR-granted antibodies, etc.) that specifically bind to a given antigen. In some embodiments, “antibody” and/or “immunoglobulin” (Ig) refers to a polypeptide comprising at least two heavy (H) chains (about 50-70 kDa) and two light (L) chains (about 25 kDa), optionally inter-connected by disulfide bonds. There are two types of light chain: λ and κ. In humans, λ and κ light chains are similar, but only one type is present in each antibody. Heavy chains are classified as mu, delta, gamma, alpha, or epsilon, and define the antibody's isotype as IgM, IgD, IgG, IgA, and IgE, respectively. See generally, Fundamental Immunology Ch. 7 (Paul, W., ed., 2nd ed. Raven Press, N.Y. (1989)) (incorporated by reference in its entirety). In some embodiments, the antibody has effector function and can fix complement. In some embodiments, the antibody has reduced or no ability to bind an Fc receptor. For example, the antibody can be an isotype or subtype, an antibody fragment or mutant, which does not support binding to an Fc receptor, e.g., it has a mutagenized or deleted Fc receptor binding region.

An “antigen-binding fragment” of an antibody refers to a fragment of a full-length antibody that retains the ability to specifically bind to an antigen (preferably with substantially the same binding affinity), or that, in other words, does not comprise a full-length antibody polypeptide, but that still comprises at least a portion of a full-length antibody polypeptide that is capable of binding to an antigen. Antibody fragments can comprise a cleaved portion of a full-length antibody polypeptide, although the term is not limited to such cleaved fragments. Examples of an antigen-binding fragment includes (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR), disulfide-linked Fvs (dsFv), and anti-idiotypic (anti-Id) antibody and intrabody. Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv)); see e.g., Bird et al. Science 242:423-426 (1988) and Huston et al., Proc. Natl. Acad. Sci. USA 85:5879-5883 (1988)). Other forms of single chain antibodies, such as diabodies, are also encompassed. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen-binding sites (see e.g., Holliger et al. Proc. Natl. Acad. Sci. USA 90:6444-6448 (1993); Poljak et al., 1994, Structure 2:1121-1123). Antibody fragments that are useful include, for example, Fab fragments, F(ab′)2 fragments, scFv (single-chain Fv) fragments, linear antibodies, monospecific or multispecific antibody fragments such as bispecific, trispecific, tetraspecific and multispecific antibodies (e.g., diabodies, triabodies, tetrabodies), monovalent or multivalent antibody fragments such as bivalent, trivalent, tetravalent and multivalent antibodies, minibodies, chelating recombinant antibodies, tribodies or bibodies, intrabodies, NANOBODY® molecules, small modular immunopharmaceuticals (SMIP), binding-domain immunoglobulin fusion proteins, camelized antibodies, and VHH containing antibodies. Additional examples of antigen-binding antibody fragments are known in the art.

The term “immunoglobulin single variable domain” (ISV), interchangeably used with “single variable domain”, defines immunoglobulin molecules wherein the antigen binding site is present on, and formed by, a single immunoglobulin domain. This sets immunoglobulin single variable domains apart from “conventional” immunoglobulins (e.g. monoclonal antibodies) or their fragments (such as Fab, Fab′, F(ab′)₂, scFv, di-scFv), wherein two immunoglobulin domains, in particular two variable domains, interact to form an antigen binding site. Typically, in conventional immunoglobulins, a heavy chain variable domain (V_{H}) and a light chain variable domain (V_{L}) interact to form an antigen binding site. In this case, the complementarity determining regions (CDRs) of both V_{H} and V_{L} will contribute to the antigen binding site, i.e. a total of 6 CDRs will be involved in antigen binding site formation.

In view of the above definition, the antigen-binding domain of a conventional 4-chain antibody (such as an IgG, IgM, IgA, IgD or IgE molecule; known in the art) or of a Fab fragment, a F(ab′)₂ fragment, an Fv fragment such as a disulfide linked Fv or a scFv fragment, or a diabody (all known in the art) derived from such conventional 4-chain antibody, would normally not be regarded as an immunoglobulin single variable domain, as, in these cases, binding to the respective epitope of an antigen would normally not occur by one (single) immunoglobulin domain but by a pair of (associating) immunoglobulin domains such as light and heavy chain variable domains, i.e., by a V_{H}-V_{L} pair of immunoglobulin domains, which jointly bind to an epitope of the respective antigen.

In contrast, immunoglobulin single variable domains are capable of specifically binding to an epitope of the antigen without pairing with an additional immunoglobulin variable domain. The binding site of an immunoglobulin single variable domain is formed by a single V_{H}, a single V_{HH} or single V_{L} domain.

As such, the single variable domain may be a light chain variable domain sequence (e.g., a V_{L}-sequence) or a suitable fragment thereof; or a heavy chain variable domain sequence (e.g., a V_{H}-sequence or V_{HH} sequence) or a suitable fragment thereof; as long as it is capable of forming a single antigen binding unit (i.e., a functional antigen binding unit that essentially consists of the single variable domain, such that the single antigen binding domain does not need to interact with another variable domain to form a functional antigen binding unit).

An immunoglobulin single variable domain (ISV) can for example be a heavy chain ISV, such as a V_{H}, V_{HH}, including a camelized V_{H} or humanized V_{HH}. In one embodiment, it is a V_{HH}, including a camelized V_{H} or humanized V_{HH}. Heavy chain ISVs can be derived from a conventional four-chain antibody or from a heavy chain antibody.

For example, the immunoglobulin single variable domain may be a single domain antibody (or an amino acid sequence that is suitable for use as a single domain antibody), a “dAb” or dAb (or an amino acid sequence that is suitable for use as a dAb) or a NANOBODY® ISV (as defined herein and including but not limited to a V_{HH}); other single variable domains, or any suitable fragment of any one thereof.

In particular, the immunoglobulin single variable domain may be a NANOBODY® ISV (such as a V_{HH}, including a humanized V_{HH} or camelized V_{H}) or a suitable fragment thereof. [Note: NANOBODY® and NANOBODIES® are registered trademarks of Ablynx N.V.]

“V_{HH} domains”, also known as V_{HH}s, V_{HH} antibody fragments, and V_{HH} antibodies, have originally been described as the antigen binding immunoglobulin variable domain of “heavy chain antibodies” (i.e., of “antibodies devoid of light chains”; Hamers-Casterman et al. Nature 363: 446-448, 1993). The term “V_{HH} domain” has been chosen in order to distinguish these variable domains from the heavy chain variable domains that are present in conventional 4-chain antibodies (which are referred to herein as “V_{H} domains”) and from the light chain variable domains that are present in conventional 4-chain antibodies (which are referred to herein as “V_{L} domains”). For a further description of V_{HH}'s, reference is made to the review article by Muyldermans (Reviews in Molecular Biotechnology 74: 277-302, 2001).

The generation of immunoglobulin sequences, such as VHHs, has been described extensively in various publications, among which WO 94/04678, Hamers-Casterman et al. 1993 and Muyldermans et al. 2001 (Reviews in Molecular Biotechnology 74: 277-302, 2001). In these methods, camelids are immunized with the target antigen in order to induce an immune response against said target antigen. The repertoire of VHHs obtained from said immunization is further screened for VHHs that bind the target antigen.

In these instances, the generation of antibodies requires purified antigen for immunization and/or screening. Antigens can be purified from natural sources, or in the course of recombinant production. Immunization and/or screening for immunoglobulin sequences can be performed using peptide fragments of such antigens.

Immunoglobulin sequences of different origin, comprising mouse, rat, rabbit, donkey, human and camelid immunoglobulin sequences can be sequenced in the method described herein. Also, fully human, humanized or chimeric sequences can be sequenced in the method described herein. For example, camelid immunoglobulin sequences and humanized camelid immunoglobulin sequences, or camelized domain antibodies, e.g. camelized dAb as described by Ward et al (see for example WO 94/04678 and Riechmann, Febs Lett., 339:285-290, 1994 and Prot. Eng., 9:531-537, 1996) can be sequenced in the method described herein. Moreover, the ISVs are fused forming a multivalent and/or multispecific construct (for multivalent and multispecific polypeptides containing one or more V_{HH} domains and their preparation, reference is also made to Conrath et al., J. Biol. Chem., Vol. 276, 10. 7346-7350, 2001, as well as to for example WO 96/34103 and WO 99/23221).

A “humanized V_{HH}” comprises an amino acid sequence that corresponds to the amino acid sequence of a naturally occurring V_{HH} domain, but that has been “humanized”, i.e. by replacing one or more amino acid residues in the amino acid sequence of said naturally occurring V_{HH} sequence (and in particular in the framework sequences) by one or more of the amino acid residues that occur at the corresponding position(s) in a V_{H} domain from a conventional 4-chain antibody from a human being (e.g. indicated above). This can be performed in a manner known per se, which will be clear to the skilled person, for example on the basis of the prior art (e.g. WO 2008/020079). Again, it should be noted that such humanized V_{HH}s can be obtained in any suitable manner known per se and thus are not strictly limited to polypeptides that have been obtained using a polypeptide that comprises a naturally occurring VHH domain as a starting material.

A “camelized V_{H}” comprises an amino acid sequence that corresponds to the amino acid sequence of a naturally occurring V_{H} domain, but that has been “camelized”, i.e. by replacing one or more amino acid residues in the amino acid sequence of a naturally occurring V_{H} domain from a conventional 4-chain antibody by one or more of the amino acid residues that occur at the corresponding position(s) in a V_{HH} domain of a (camelid) heavy chain antibody. This can be performed in a manner known per se, which will be clear to the skilled person, for example on the basis of the description in the prior art (e.g. Davies and Riechman (1994 and 1996), supra). Such “camelizing” substitutions are inserted at amino acid positions that form and/or are present at the V_{H}-V_{L} interface, and/or at the so-called Camelidae hallmark residues, as defined herein (see for example WO 94/04678 and Davies and Riechmann (1994 and 1996), supra). In one embodiment, the V_{H} sequence that is used as a starting material or starting point for generating or designing the camelized V_{H} is a V_{H} sequence from a mammal, such as the V_{H} sequence of a human being, such as a V_{H}3 sequence. However, it should be noted that such camelized V_{H} can be obtained in any suitable manner known per se and thus are not strictly limited to polypeptides that have been obtained using a polypeptide that comprises a naturally occurring V_{H} domain as a starting material.

The structure of an immunoglobulin single variable domain sequence can be considered to be comprised of four framework regions (“FRs”), which are referred to in the art and herein as “Framework region 1” (“FR1”); as “Framework region 2” (“FR2”); as “Framework region 3” (“FR3”); and as “Framework region 4” (“FR4”), respectively; which framework regions are interrupted by three complementary determining regions (“CDRs”), which are referred to in the art and herein as “Complementarity Determining Region 1” (“CDR1”); as “Complementarity Determining Region 2” (“CDR2”); and as “Complementarity Determining Region 3” (“CDR3”), respectively.

In such an immunoglobulin sequence, the framework sequences may be any suitable framework sequences, and examples of suitable framework sequences will be clear to the skilled person, for example on the basis the standard handbooks and the further disclosure and prior art mentioned herein.

The framework sequences are (a suitable combination of) immunoglobulin framework sequences or framework sequences that have been derived from immunoglobulin framework sequences (for example, by humanization or camelization). For example, the framework sequences may be framework sequences derived from a light chain variable domain (e.g. a V_{L}-sequence) and/or from a heavy chain variable domain (e.g. a V_{H}-sequence or V_{HH} sequence). In one particular aspect, the framework sequences are either framework sequences that have been derived from a V_{HH}-sequence (in which said framework sequences may optionally have been partially or fully humanized) or are conventional V_{H} sequences that have been camelized (as defined herein).

In particular, the framework sequences present in the ISV sequence used in the methods described herein may contain one or more of hallmark residues (as defined herein), such that the ISV sequence is a NANOBODY® ISV, such as e.g. a V_{HH}, including a humanized V_{HH} or camelized V_{H}. Non-limiting examples of (suitable combinations of) such framework sequences will become clear from the further disclosure herein.

The total number of amino acid residues in a V_{H} domain and a V_{HH} domain will usually be in the range of from 110 to 120, often between 112 and 115. It should however be noted that smaller and longer sequences may also be suitable for the purposes described herein.

However, it should be noted that the ISVs comprised in the multivalent ISV polypeptide that is sequenced in the present method is not limited as to the origin of the ISV sequence (or of the nucleotide sequence used to express it), nor as to the way that the ISV sequence or nucleotide sequence is (or has been) generated or obtained. Thus, the ISV sequences may be naturally occurring sequences (from any suitable species) or synthetic or semi-synthetic sequences. In a specific but non-limiting aspect, the ISV sequence is a naturally occurring sequence (from any suitable species) or a synthetic or semi-synthetic sequence, including but not limited to “humanized” (as defined herein) immunoglobulin sequences (such as partially or fully humanized mouse or rabbit immunoglobulin sequences, and in particular partially or fully humanized V_{HH} sequences), “camelized” (as defined herein) immunoglobulin sequences (and in particular camelized V_{H} sequences), as well as ISVs that have been obtained by techniques such as affinity maturation (for example, starting from synthetic, random or naturally occurring immunoglobulin sequences), CDR grafting, veneering, combining fragments derived from different immunoglobulin sequences, PCR assembly using overlapping primers, and similar techniques for engineering immunoglobulin sequences well known to the skilled person; or any suitable combination of any of the foregoing.

Similarly, nucleotide sequences may be naturally occurring nucleotide sequences or synthetic or semi-synthetic sequences, and may for example be sequences that are isolated by PCR from a suitable naturally occurring template (e.g. DNA or RNA isolated from a cell), nucleotide sequences that have been isolated from a library (and in particular, an expression library), nucleotide sequences that have been prepared by introducing mutations into a naturally occurring nucleotide sequence (using any suitable technique known per se, such as mismatch PCR), nucleotide sequence that have been prepared by PCR using overlapping primers, or nucleotide sequences that have been prepared using techniques for DNA synthesis known per se.

Generally, NANOBODY® ISVs (in particular V_{HH} sequences, including (partially) humanized V_{HH} sequences and camelized V_{H} sequences) can be characterized by the presence of one or more “Hallmark residues” (as described herein) in one or more of the framework sequences (again as further described herein). Thus, generally, a NANOBODY® ISV can be defined as an immunoglobulin sequence with the (general) structure

FR1-CDR1-FR2-CDR2-FR3-CDR3-FR4

in which FR1 to FR4 refer to framework regions 1 to 4, respectively, and in which CDR1 to CDR3 refer to the complementarity determining regions 1 to 3, respectively, and in which one or more of the Hallmark residues are as further defined herein.
In particular, a NANOBODY® ISV can be an immunoglobulin sequence with the (general) structure

FR1-CDR1-FR2-CDR2-FR3-CDR3-FR4

in which FR1 to FR4 refer to framework regions 1 to 4, respectively, and in which CDR1 to CDR3 refer to the complementarity determining regions 1 to 3, respectively, and in which the framework sequences are as further defined herein.
More in particular, a NANOBODY® ISV can be an immunoglobulin sequence with the (general) structure

FR1-CDR1-FR2-CDR2-FR3-CDR3-FR4

in which FR1 to FR4 refer to framework regions 1 to 4, respectively, and in which CDR1 to CDR3 refer to the complementarity determining regions 1 to 3, respectively, and in which: one or more of the amino acid residues at positions 11, 37, 44, 45, 47, 83, 84, 103, 104 and 108 according to the Kabat numbering are chosen from the Hallmark residues mentioned in Table 4 below.

TABLE 4
Hallmark Residues in NANOBODY ® ISVs

| Position | Human V_{H}3 | Hallmark Residues |
|---|---|---|
| 11 | L, V; predominantly L | L, S, V, M, W, F, T, Q, E, A, R, G, K, Y, N, P, I; |
| | | preferably L |
| 37 | V, I, F; usually V | F⁽¹⁾, Y, V, L, A, H, S, I, W, C, N, G, D, T, P, preferably |
| | | F⁽¹⁾ or Y |
| 44⁽⁸⁾ | G | E⁽³⁾, Q⁽³⁾, G⁽²⁾, D, A, K, R, L, P, S, V, H, T, N, W, M, I; |
| | | preferably G⁽²⁾, E⁽³⁾ or Q⁽³⁾; most preferably G⁽²⁾ or |
| | | Q⁽³⁾. |
| 45⁽⁸⁾ | L | L⁽²⁾, R⁽³⁾, P, H, F, G, Q, S, E, T, Y, C, I, D, V; |
| | | preferably L⁽²⁾ or R⁽³⁾ |
| 47⁽⁸⁾ | W, Y | F⁽¹⁾, L⁽¹⁾ or W⁽²⁾ G, I, S, A, V, M, R, Y, E, P, T, C, H, K, |
| | | Q, N, D; preferably W⁽²⁾, L⁽¹⁾ or F⁽¹⁾ |
| 83 | R or K; usually R | R, K⁽⁵⁾, T, E⁽⁵⁾, Q, N, S, I, V, G, M, L, A, D, Y, H; |
| | | preferably K or R; most preferably K |
| 84 | A, T, D; predominantly A | P⁽⁵⁾, S, H, L, A, V, I, T, F, D, R, Y, N, Q, G, E; |
| | | preferably P |
| 103 | W | W⁽⁴⁾, R⁽⁶⁾, G, S, K, A, M, Y, L, F, T, N, V, Q, P⁽⁶⁾, E, C; |
| | | preferably W |
| 104 | G | G, A, S, T, D, P, N, E, C, L; preferably G |
| 108 | L, M or T; predominantly | Q, L⁽⁷⁾, R, P, E, K, S, T, M, A, H; preferably Q or L⁽⁷⁾ |
| | L | |

Notes:
In particular, but not exclusively, in combination with KERE or KQRE at positions 43-46.
Usually as GLEW at positions 44-47.
Usually as KERE or KQRE at positions 43-46, e.g. as KEREL, KEREF, KQREL, KQREF, KEREG, KQREW or KQREG at positions 43-47. Alternatively, also sequences such as TERE (for example TEREL), TQRE (for example TQREL), KECE (for example KECEL or KECER), KQCE (for example KQCEL), RERE (for example REREG), RQRE (for example RQREL, RQREF or RQREW), QERE (for example QEREG), QQRE, (for example QQREW, QQREL or QQREF), KGRE (for example KGREG), KDRE (for example KDREV) are possible. Some other possible, but less preferred sequences include for example DECKL and NVCEL.
Often as KP or EP at positions 83-84 of naturally occurring V_{HH} domains.
In particular, but not exclusively, in combination with GLEW at positions 44-47.
With the proviso that when positions 44-47 are GLEW, position 108 is always Q in (non-humanized) V_{HH} sequences that also contain a W at 103.
The GLEW group also contains GLEW-like sequences at positions 44-47, such as for example GVEW, EPEW, GLER, DQEW, DLEW, GIEW, ELEW, GPEW, EWLP, GPER, GLER and ELEW.

The terms “microRNA,” “miRNA” and “miR” are used interchangeably herein and refer to a single stranded non-coding RNA that functions in RNA silencing and post-transcriptional regulation of gene expression. miRNAs function by complementary base pairing with mRNA molecules, that silences the mRNA, by, inter alia, one or more of the following: (a) cleavage of the mRNA strand into two pieces, (b) destabilization of the mRNA through shortening of its poly(A) tail, and (c) less efficient translation of the mRNA into proteins by ribosomes.

The terms “subject,” “patient” and “individual” are used interchangeably and refer to mammals including, but not limited to, human patients and non-human primates, as well as experimental animals such as rabbits, dogs, cats, rats, mice, and other animals. Accordingly, the term “subject” or “patient” as used herein means any mammalian patient or subject to which the compositions of the disclosure can be administered. Subjects include those with a disorder. In one embodiment, the subject is a human.

The term “genetic disease” as used herein refers to a disease or disorder that is treatable with a polynucleotide therapeutic. Examples of genetic diseases include, but are not limited to any disease or disorder caused by a genetic mutation, cancers, and viral infections, diseases or disorders caused by a mutation that may be corrected using gene editing (e.g., CRISPR/Cas9, or Zinc Finger Nucleases), diseases or disorders caused by overexpression of a gene, and diseases or disorders caused by decreased or lack of expression of a gene.

The term “targeting molecule” as used herein refers to a molecule that binds or localizes at a particular target or location. The molecule may be for example, be an antibody or an antigen-binding fragment thereof, or a binding protein. The targeting molecule may be, for example, an immunoglobulin single variable domain (ISV) such as a NANOBODY® molecule.

A pharmaceutical composition can comprise a first active ingredient. The pharmaceutical composition can be formulated in unit dose form. The pharmaceutical composition can comprise a pharmaceutically acceptable excipient, diluent, or carrier. The pharmaceutical composition can comprise a second, third, or fourth active ingredient.

A composition described herein can compromise an excipient. An excipient can comprise a pH agent (to minimize oxidation or degradation of a component of the composition), a stabilizing agent (to prevent modification or degradation of a component of the composition), a buffering agent (to enhance temperature stability), a solubilizing agent (to increase protein solubility), or any combination thereof. An excipient can comprise a surfactant, a sugar, an amino acid, an antioxidant, a salt, a non-ionic surfactant, a solubilizer, a triglyceride, an alcohol, or any combination thereof. An excipient can comprise sodium carbonate, acetate, citrate, phosphate, polyethylene glycol (PEG), human serum albumin (HSA), sorbitol, sucrose, trehalose, polysorbate 80, sodium phosphate, sucrose, disodium phosphate, mannitol, polysorbate 20, histidine, citrate, albumin, sodium hydroxide, glycine, sodium citrate, trehalose, arginine, sodium acetate, acetate, HCl, disodium edetate, lecithin, glycerin, xanthan rubber, soy isoflavones, polysorbate 80, ethyl alcohol, water, teprenone, or any combination thereof. An excipient can be an excipient described in the Handbook of Pharmaceutical Excipients, American Pharmaceutical Association (1986).

The terms “pharmaceutically effective amount,” “therapeutically effective amount,” or “therapeutically effective dose” refer to an amount effective to treat a disease in a patient, e.g., effecting a beneficial and/or desirable alteration in the general health of a patient suffering from a disease (e.g., a genetic disease as described herein), treatment, healing, inhibition or amelioration of a physiological response or condition, etc. The full therapeutic effect does not necessarily occur by administration of one dose, and may occur only after administration of a series of doses. Thus, a therapeutically effective amount may be administered in one or more administrations. The precise effective amount needed for a subject will depend upon, for example, the subject's size, health and age, the nature and extent of disease, the therapeutics or combination of therapeutics selected for administration, and the mode of administration. The skilled worker can readily determine the effective amount for a given situation by routine experimentation. The skilled worker will recognize that, for example, treating cancer includes, but is not limited to, killing cancer cells, preventing the growth of new cancer cells, causing tumor regression (a decrease in tumor size), causing a decrease in metastasis, improving vital functions of a patient, improving the well-being of the patient, decreasing pain, improving appetite, improving the patient's weight, and any combination thereof. The terms “pharmaceutically effective amount,” “therapeutically effective amount,” or (therapeutically effective dose” also refer to the amount required to improve the clinical symptoms of a patient. The therapeutic methods or methods of treating described herein are not to be interpreted or otherwise limited to “curing” the disease.

As used herein, the term “treating” or “treatment” includes reversing, reducing, or arresting the symptoms, clinical signs, and underlying pathology of a condition in manner to improve or stabilize a subject's condition. As used herein, and as well understood in the art, “treatment” is an approach for obtaining beneficial or desired results, including clinical results. Beneficial or desired clinical results can include, but are not limited to, alleviation, amelioration, or slowing the progression, of one or more symptoms or conditions associated with a condition, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Exemplary beneficial clinical results are described herein. The terms “treating” and “treatment” may also relate to reduction in severity and/or frequency of symptoms, elimination of symptoms and/or underlying cause, prevention of the occurrence of symptoms and/or their underlying cause, and improvement or remediation of damage.

“Administering” or “administration of” a composition as disclosed herein to a subject can be carried out using one of a variety of methods known to those skilled in the art. Administering can also be performed, for example, once, a plurality of times, and/or over one or more extended periods. In some aspects, the administration includes both direct administration, including self-administration, and indirect administration, including the act of prescribing a drug. For example, as used herein, a physician who instructs a patient to self-administer a drug, or to have the drug administered by another and/or who provides a patient with a prescription for a drug is administering the drug to the patient. When a method is part of a therapeutic regimen involving more than one pharmaceutical composition or treatment modality, the disclosure contemplates that the pharmaceutical compositions may be administered at the same or differing times and via the same or differing routes of administration.

Administration or application of a composition disclosed herein can be performed for a treatment duration of at least about at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 days consecutive or nonconsecutive days. In some cases, a treatment duration can be from about 1 to about 30 days, from about 2 to about 30 days, from about 3 to about 30 days, from about 4 to about 30 days, from about 5 to about 30 days, from about 6 to about 30 days, from about 7 to about 30 days, from about 8 to about 30 days, from about 9 to about 30 days, from about 10 to about 30 days, from about 11 to about 30 days, from about 12 to about 30 days, from about 13 to about 30 days, from about 14 to about 30 days, from about 15 to about 30 days, from about 16 to about 30 days, from about 17 to about 30 days, from about 18 to about 30 days, from about 19 to about 30 days, from about 20 to about 30 days, from about 21 to about 30 days, from about 22 to about 30 days, from about 23 to about 30 days, from about 24 to about 30 days, from about 25 to about 30 days, from about 26 to about 30 days, from about 27 to about 30 days, from about 28 to about 30 days, or from about 29 to about 30 days.

Administration or application of a composition disclosed herein can be performed for a treatment duration of at least about 1 week, at least about 1 month, at least about 1 year, at least about 2 years, at least about 3 years, at least about 4 years, at least about 5 years, at least about 6 years, at least about 7 years, at least about 8 years, at least about 9 years, at least about 10 years, at least about 15 years, at least about 20 years, or more. Administration can be performed repeatedly over a lifetime of a subject, such as once a month or once a year for the lifetime of a subject. Administration can be performed repeatedly over a substantial portion of a subject's life, such as once a month or once a year for at least about 1 year, 5 years, 10 years, 15 years, 20 years, 25 years, 30 years, or more.

Administration or application of composition disclosed herein can be performed at least 1, 2, 3, 4, 5, 6, 7, 8, 9, 10, 11, 12, 13, 14, 15, 16, 17, 18, 19, 20, 21, 22, 23, or 24 times a day. In some cases, administration or application of composition disclosed herein can be performed at least 1, 2, 3, 4, 5, 6, 7, 8, 9, 10, 11, 12, 13, 14, 15, 16, 17, 18, 19, 20, or 21 times a week. In some cases, administration or application of composition disclosed herein can be performed at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 times a month.

In some cases, a composition can be administered/applied as a single dose or as divided doses. In some cases, the compositions described herein can be administered at a first time point and a second time point. In some cases, a composition can be administered such that a first administration is administered before the other with a difference in administration time of 1 hour, 2 hours, 4 hours, 8 hours, 12 hours, 16 hours, 20 hours, 1 day, 2 days, 4 days, 7 days, 2 weeks, 4 weeks, 2 months, 3 months, 4 months, 5 months, 6 months, 7 months, 8 months, 9 months, 10 months, 11 months, 1 year or more.

As used herein, a second therapy can include immunotherapy, hormone therapy, cryotherapy, surgical procedure or any combination thereof. A second therapy can include administration of a pharmaceutical composition, such as a small molecule. A second therapy can include administration of a pharmaceutical composition, such as one or more antibiotics. A second therapy can comprise administration of a muscle relaxant, an anti-depressant, a steroid, an opioid, a cannabis-based therapeutic, acetaminophen, a non-steroidal anti-inflammatory, a neuropathic agent, a cannabis, a progestin, a progesterone, or any combination thereof. A neuropathic agent may comprise gabapentin. A non-steroidal anti-inflammatory can comprise naproxen, ibuprofen, a COX-2 inhibitor, or any combination thereof. A second therapy can comprise administration of a biologic agent, cellular therapy, regenerative medicine therapy, a tissue engineering approach, a stem cell transplantation or any combination thereof. A second therapy can comprise a medical procedure. A medical procedure can comprise an epidural injection (such as a steroid injection), acupuncture, exercise, physical therapy, an ultrasound, a surgical therapy, a chiropractic manipulation, an osteopathic manipulation, a chemonucleolysis, or any combination thereof. A second therapy can comprise use of a breathing assist device or a ventilator. A second therapy can comprise administration of a regenerative therapy or an immunotherapy such as a protein, a stem cell, a cord blood cell, an umbilical cord tissue, a tissue, or any combination thereof. A second therapy can comprise a biosimilar.

A diagnostic test can comprise an imaging procedure, a blood count analysis, a tissue pathology analysis, a biomarker analysis, a biopsy, a magnetic resonance image procedure, a physical examination, a urine test, an ultrasonography procedure, a genetic test, a liver function test, a positron emission tomography procedure, a X-ray, serology, an angiography procedure, an electrocardiography procedure, an endoscopy, a diagnostic polymerase chain reaction test (PCR), a pap smear, a hematocrit test, a skin allergy test, a urine test, a colonoscopy, an enzyme-linked immunosorbent assay (ELISA), microscopy analysis, bone marrow examination, rapid diagnostic test, pregnancy test, organ function test, toxicology test, infectious disease test, bodily fluids test, or any combination thereof.

The term “tissue” as used herein, can be any tissue sample. A tissue can be a tissue suspected or confirmed of having a disease or condition. A tissue can be a sample that may be substantially healthy, substantially benign, or otherwise substantially free of a disease or a condition. A tissue can be a tissue removed from a subject, such as a tissue biopsy, a tissue resection, an aspirate (such as a fine needle aspirate), a tissue washing, a cytology specimen, a bodily fluid, or any combination thereof. A tissue can comprise cancerous cells, tumor cells, non-cancerous cells, or a combination thereof. A tissue can comprise a blood sample (such as a cell-free DNA sample). A tissue can be a sample that may be genetically modified.

In some cases, a disease or condition can comprise a neuromuscular disorder.

In some embodiments, a polynucleotide is conjugated to a (therapeutic) targeting molecule/agent. In some embodiments, the targeting moiety comprises amino acids, peptides, polypeptides, proteins, antibodies, antigens, toxins, hormones, lipids, nucleotides, nucleosides, sugars, carbohydrates, polymers such as polyethylene glycol and polypropylene glycol, as well as analogs or derivatives of all of these classes of substances. Additional examples of targeting moiety also include steroids, such as cholesterol, phospholipids, di- and triacylglycerols, fatty acids, hydrocarbons (e.g., saturated, unsaturated, or contains substitutions), enzyme substrates, biotin, digoxigenin, and polysaccharides. In some embodiments, the targeting moiety is an antibody or binding fragment thereof. Targeting moiety is used interchangeably with targeting agent or targeting molecule herein.

The targeting molecule may be an antibody or an antigen-binding fragment thereof, or a binding protein. In some embodiments, the targeting molecule is an antibody or an antigen binding fragment thereof (e.g. a polynucleotide-antibody conjugate). In some embodiments, the antibody or binding fragment thereof is a human antibody or an antigen-binding fragment thereof, a humanized antibody or an antigen-binding fragment thereof, a murine antibody or an antigen-binding fragment thereof, a chimeric antibody or an antigen-binding fragment thereof, a monoclonal antibody or an antigen-binding fragment thereof, a monovalent Fab′, a divalent Fab2, a F(ab)′3 fragment, a single-chain variable fragment (scFv), a bis-scFv, a (scFv)2, a diabody, a minibody, a immunoglobulin single variable domain (ISV) such as an NANOBODY® molecule, a triabody, a tetrabody, a disulfide stabilized Fv protein (dsFv), a single-domain antibody (sdAb), an Ig NAR, a vNAR, a mutein based on Tenascin C (also known as Centyrin molecule), a camelid antibody or an antigen-binding fragment thereof, a bispecific antibody or an antigen binding fragment thereof, or a chemically modified derivative thereof. In some embodiments, the antibody or antigen-binding fragment thereof is selected from the group consisting of a monoclonal antibody, a bispecific antibody, a Fab, a Fab-Fc, a Fv, a single chain Fv (scFv), a diabody, a minibody, and a immunoglobulin single variable domain (ISV) such as an NANOBODY® molecule. In some embodiments, the antibody or antigen-binding fragment thereof is a monoclonal antibody. In some embodiments, the antibody or antigen-binding fragment thereof is a bispecific antibody. Non-limiting examples of bispecific antibodies in bispecific T-cell engagers (BiTEs) and a dual-affinity retargeting antibodies (DARTs). In some embodiments, the bispecific antibody is a trifunctional antibody or a bispecific mini-antibody. In some embodiments, the bispecific antibody is a trifunctional antibody. In some embodiments, the trifunctional antibody is a full-length monoclonal antibody comprising binding sites for two different antigens. In some embodiments, the bispecific antibody is a bispecific mini-antibody. In some embodiments, the bispecific mini-antibody comprises divalent Fab2, F(ab)′3 fragments, bis-scFv, (scFv)2, diabody, minibody, triabody, tetrabody or a bi-specific T-cell engager (BiTE). In some embodiments, the bi-specific T-cell engager is a fusion protein that contains two single-chain variable fragments (scFvs) in which the two scFvs target epitopes of two different antigens.

In some embodiments, the antibody or antigen-binding fragment thereof is a Fab. In some embodiments, the antibody or antigen-binding fragment thereof is a Fab-Fc. In some embodiments, the antibody or antigen-binding fragment thereof is a Fv. In some embodiments, the antibody or antigen-binding fragment thereof is a single chain Fv (scFv). In some embodiments, when the antibody or antigen-binding portion is a scFv, the polynucleotide does not comprise a cross-linking residue. In some embodiments, when the antibody or antigen-binding portion is a scFv, the polynucleotide does not comprise a cysteine. In some embodiments, the antibody or antigen-binding fragment thereof is a diabody. In some embodiments, the antibody or antigen-binding fragment thereof is a minibody. In some embodiments, the antibody or antigen-binding fragment thereof is a immunoglobulin single variable domain (ISV) such as an NANOBODY® molecule. The NANOBODY® molecule may be a NANOBODY® molecule-HSA.

In some embodiments, the antibody or antigen-binding fragment thereof is an IgG molecule or is derived from an IgG molecule. The IgG molecule may be an IgG1 or an IgG4 molecule. The antibody or antigen-binding fragment thereof may be an IgG1 molecule or derived therefrom. The antibody or antigen-binding fragment thereof may be an IgG2 molecule or derived therefrom. The antibody or antigen-binding fragment thereof may be an IgG3 molecule or derived therefrom. The antibody or antigen-binding fragment thereof may be an IgG4 molecule or derived therefrom.

In some embodiments, the targeting molecule is a binding protein. The binding protein may be a soluble receptor or a soluble ligand. In some embodiments, the soluble receptor comprises the extracellular domain of a receptor. In some embodiments, the soluble receptor is a Fc fusion protein.

In some embodiments, the targeting molecule is a plasma protein. In some embodiments, the plasma protein comprises albumin. In some embodiments, the albumin is conjugated by one or more of the conjugation chemistries disclosed herein to a polynucleotide. In some instances, the albumin is conjugated by native ligation chemistry to a polynucleotide. In some instances, albumin is conjugated by lysine conjugation to a polynucleotide.

In some instances, the targeting molecule is a steroid. Non-limiting exemplary steroids include cholesterol, phospholipids, di- and triacylglycerols, fatty acids, hydrocarbons that are saturated, unsaturated, comprise substitutions, or combinations thereof. In some embodiments, the steroid is cholesterol or a cholesterol derivative. In some embodiments, the targeting molecule is cholesterol. In some embodiments, the steroid is conjugated by one or more of the conjugation chemistries disclosed herein to a polynucleotide. In some embodiments, the steroid is conjugated by native ligation chemistry to a polynucleotide.

In some embodiments, the targeting molecule is a polymer, including but not limited to polynucleotide aptamers that bind to specific surface markers on cells. In some embodiments, the targeting molecule is a polynucleotide that does not hybridize to a target gene or mRNA, but instead is capable of selectively binding to a cell surface marker similarly to an antibody binding to its specific epitope of a cell surface marker.

In some embodiments, the targeting molecule comprises or is a polypeptide. In some embodiments, the polypeptide has a size between about 1 and about 3 kDa. In some embodiments, the polypeptide has a size between about 1.2 and about 2.8 kDa, between about 1.5 and about 2.5 kDa, or between about 1.5 and about 2 kDa. In some embodiments, the targeting molecule is a polypeptide. In some embodiments, the polypeptide has a size between 1 and 3 kDa. In some embodiments, the polypeptide has a size between 1.2 and 2.8 kDa, between 1.5 and 2.5 kDa, or between 1.5 and 2 kDa. In some embodiments, the polypeptide is a bicyclic polypeptide. In some embodiments, the bicyclic polypeptide is a constrained bicyclic polypeptide. In some embodiments, the targeting molecule is a bicyclic polypeptide (e.g., bicycles from Bicycle Therapeutics).

In additional embodiments, the targeting molecule comprises or is a small molecule. In some embodiments, the small molecule comprises or is an antibody-recruiting small molecule. In some embodiments, the antibody-recruiting small molecule comprises a target-binding terminus and an antibody-binding terminus, in which the target-binding terminus is capable of recognizing and interacting with a cell surface receptor.

In some embodiments, the targeting molecule comprises or is a therapeutically active molecule or a biologically active molecule.

In some embodiment, the agent (such as a therapeutic agent or therapeutic targeting agent described herein) comprises a polynucleotide.

In some embodiments, the polynucleotide is from about 5 to about 100 nucleotides in length. In some embodiments, the polynucleotide is from about 5 to about 50 nucleotides in length. In some embodiments, the polynucleotide is from about 10 to about 30, from about 15 to about 30, from about 18 to about 25, from about 18 to about 24, from about 19 to about 23, or from about 20 to about 22 nucleotides in length. In some embodiments, the polynucleotide is about 50 nucleotides in length. In some embodiments, the polynucleotide is about 49 nucleotides in length. In some embodiments, the polynucleotide is about 48 nucleotides in length. In some embodiments, the polynucleotide is about 47 nucleotides in length. In some embodiments, the polynucleotide is about 46 nucleotides in length. In some embodiments, the polynucleotide is about 45 nucleotides in length. In some embodiments, the polynucleotide is about 44 nucleotides in length. In some embodiments, the polynucleotide is about 43 nucleotides in length. In some embodiments, the polynucleotide is about 42 nucleotides in length. In some embodiments, the polynucleotide is about 41 nucleotides in length. In some embodiments, the polynucleotide is about 40 nucleotides in length. In some embodiments, the polynucleotide is about 39 nucleotides in length. In some embodiments, the polynucleotide is about 38 nucleotides in length. In some embodiments, the polynucleotide is about 37 nucleotides in length. In some embodiments, the polynucleotide is about 36 nucleotides in length. In some embodiments, the polynucleotide is about 35 nucleotides in length. In some embodiments, the polynucleotide is about 34 nucleotides in length. In some embodiments, the polynucleotide is about 33 nucleotides in length. In some embodiments, the polynucleotide is about 32 nucleotides in length. In some embodiments, the polynucleotide is about 31 nucleotides in length. In some embodiments, the polynucleotide is about 30 nucleotides in length. In some embodiments, the polynucleotide is about 29 nucleotides in length. In some embodiments, the polynucleotide is about 28 nucleotides in length. In some embodiments, the polynucleotide is about 27 nucleotides in length. In some embodiments, the polynucleotide is about 26 nucleotides in length. In some embodiments, the polynucleotide is about 25 nucleotides in length. In some embodiments, the polynucleotide is about 24 nucleotides in length. In some embodiments, the polynucleotide is about 23 nucleotides in length. In some embodiments, the polynucleotide is about 22 nucleotides in length. In some embodiments, the polynucleotide is about 21 nucleotides in length. In some embodiments, the polynucleotide is about 20 nucleotides in length. In some embodiments, the polynucleotide is about 19 nucleotides in length. In some embodiments, the polynucleotide is about 18 nucleotides in length. In some embodiments, the polynucleotide is about 17 nucleotides in length. In some embodiments, the polynucleotide is about 16 nucleotides in length. In some embodiments, the polynucleotide is about 15 nucleotides in length. In some embodiments, the polynucleotide is about 14 nucleotides in length. In some embodiments, the polynucleotide is about 13 nucleotides in length. In some embodiments, the polynucleotide is about 12 nucleotides in length. In some embodiments, the polynucleotide is about 11 nucleotides in length. In some embodiments, the polynucleotide is about 10 nucleotides in length. In some embodiments, the polynucleotide is about 9 nucleotides in length. In some embodiments, the polynucleotide is about 8 nucleotides in length. In some embodiments, the polynucleotide is about 7 nucleotides in length. In some embodiments, the polynucleotide is about 6 nucleotides in length. In some embodiments, the polynucleotide is about 5 nucleotides in length. In some embodiments, the polynucleotide is from about 10 to about 50 nucleotides in length. In some embodiments, the polynucleotide is from about 10 to about 45 nucleotides in length. In some embodiments, the polynucleotide is from about 10 to about 40 nucleotides in length. In some embodiments, the polynucleotide is from about 10 to about 35 nucleotides in length. In some embodiments, the polynucleotide is from about 10 to about 30 nucleotides in length. In some embodiments, the polynucleotide is from about 10 to about 25 nucleotides in length. In some embodiments, the polynucleotide is from about 10 to about 20 nucleotides in length. In some embodiments, the polynucleotide is from about 15 to about 25 nucleotides in length. In some embodiments, the polynucleotide is from about 15 to about 30 nucleotides in length. In some embodiments, the polynucleotide is from about 12 to about 30 nucleotides in length.

In some embodiments, the polynucleotide is from 5 to 100 nucleotides in length. In some embodiments, the polynucleotide is from 5 to 50 nucleotides in length. In some embodiments, the polynucleotide is from 10 to 30, from 15 to 30, from 18 to 25, from 18 to 24, from 19 to 23, or from 20 to 22 nucleotides in length. In some embodiments, the polynucleotide is 50 nucleotides in length. In some embodiments, the polynucleotide is 49 nucleotides in length. In some embodiments, the polynucleotide is 48 nucleotides in length. In some embodiments, the polynucleotide is 47 nucleotides in length. In some embodiments, the polynucleotide 46 nucleotides in length. In some embodiments, the polynucleotide is 45 nucleotides in length. In some embodiments, the polynucleotide is 44 nucleotides in length. In some embodiments, the polynucleotide is 43 nucleotides in length. In some embodiments, the polynucleotide is 42 nucleotides in length. In some embodiments, the polynucleotide is 41 nucleotides in length. In some embodiments, the polynucleotide is 40 nucleotides in length. In some embodiments, the polynucleotide is 39 nucleotides in length. In some embodiments, the polynucleotide is 38 nucleotides in length. In some embodiments, the polynucleotide is 37 nucleotides in length. In some embodiments, the polynucleotide is 36 nucleotides in length. In some embodiments, the polynucleotide is 35 nucleotides in length. In some embodiments, the polynucleotide is 34 nucleotides in length. In some embodiments, the polynucleotide is 33 nucleotides in length. In some embodiments, the polynucleotide is 32 nucleotides in length. In some embodiments, the polynucleotide is 31 nucleotides in length. In some embodiments, the polynucleotide is 30 nucleotides in length. In some embodiments, the polynucleotide is 29 nucleotides in length. In some embodiments, the polynucleotide is 28 nucleotides in length. In some embodiments, the polynucleotide is 27 nucleotides in length. In some embodiments, the polynucleotide is 26 nucleotides in length. In some embodiments, the polynucleotide is 25 nucleotides in length. In some embodiments, the polynucleotide is 24 nucleotides in length. In some embodiments, the polynucleotide is 23 nucleotides in length. In some embodiments, the polynucleotide is 22 nucleotides in length. In some embodiments, the polynucleotide is 21 nucleotides in length. In some embodiments, the polynucleotide is 20 nucleotides in length. In some embodiments, the polynucleotide is 19 nucleotides in length. In some embodiments, the polynucleotide is 18 nucleotides in length. In some embodiments, the polynucleotide is 17 nucleotides in length. In some embodiments, the polynucleotide is 16 nucleotides in length. In some embodiments, the polynucleotide is 15 nucleotides in length. In some embodiments, the polynucleotide is 14 nucleotides in length. In some embodiments, the polynucleotide is 13 nucleotides in length. In some embodiments, the polynucleotide is 12 nucleotides in length. In some embodiments, the polynucleotide is 11 nucleotides in length. In some embodiments, the polynucleotide is 10 nucleotides in length. In some embodiments, the polynucleotide is 9 nucleotides in length. In some embodiments, the polynucleotide is 8 nucleotides in length. In some embodiments, the polynucleotide is 7 nucleotides in length. In some embodiments, the polynucleotide is 6 nucleotides in length. In some embodiments, the polynucleotide is 5 nucleotides in length. In some embodiments, the polynucleotide is from 10 to 50 nucleotides in length. In some embodiments, the polynucleotide is from 10 to 45 nucleotides in length. In some embodiments, the polynucleotide is from 10 to 40 nucleotides in length. In some embodiments, the polynucleotide is from 10 to 35 nucleotides in length. In some embodiments, the polynucleotide is from 10 to 30 nucleotides in length. In some embodiments, the polynucleotide is from 10 to 25 nucleotides in length. In some embodiments, the polynucleotide is from 10 to 20 nucleotides in length. In some embodiments, the polynucleotide is from 15 to 25 nucleotides in length. In some embodiments, the polynucleotide is from 15 to 30 nucleotides in length. In some embodiments, the polynucleotide is from 12 to 30 nucleotides in length.

In some embodiments, the polynucleotide comprises RNA, DNA or a combination thereof. In some cases, the polynucleotide comprises RNA. In some cases, the polynucleotide comprises DNA. In some cases, the polynucleotide comprises RNA and DNA. In some embodiments, the polynucleotide comprises combinations of DNA, RNA and/or artificial nucleotide analogues. In some embodiments, the polynucleotide is a regulatory non-coding RNA (ncRNA). In some embodiments, the ncRNA comprises short non-coding RNA sequences expressed in a genome that regulates expression or function of other biomolecules in mammalian cells. An ncRNA is generally <200 nucleotides in length and can be single stranded or double stranded and may form non-linear secondary or tertiary structures. An ncRNA can comprise exogenously derived small interfering RNA (siRNA), MicroRNA (miRNA), small nuclear RNA (U-RNA), Small nucleolar RNA (snoRNA), Piwi-interacting RNA (piRNA), repeat associated small interfering RNA (rasiRNA), small rDNA-derived RNA (srRNA), transfer RNA derived small RNA (tsRNA), ribosomal RNA derived small RNA (rsRNA), large non-coding RNA derived small RNA (lncsRNA), or a messenger RNA derived small RNA (msRNA). In some embodiments, the polynucleotide is an engineered polynucleotide. The engineered polynucleotide may comprise DNA or RNA. In some embodiments, the engineered polynucleotide comprises a plurality of nucleotides. In some embodiments, the engineered polynucleotide comprises an artificial nucleotide analogue. In some embodiments, the engineered polynucleotide comprises DNA. In some embodiments, the DNA is genomic DNA, cell-free DNA, cDNA, fetal DNA, viral DNA, or maternal DNA. In some embodiments, the engineered polynucleotide comprises RNA. In some embodiments, the RNA is an siRNA, an ncRNA mimic, a short-harpin RNA (shRNA), a dicer-dependent siRNA (di-siRNA), an antisense oligonucleotide (ASO), a gapmer, a mixmer, double-stranded RNAs (dsRNA), single stranded RNAi, (ssRNAi), DNA-directed RNA interference (ddRNAi), an RNA activating oligonucleotide (RNAa), a transfer RNA (tRNA), a ribosomal RNA (rRNA), a heterogeneous nuclear RNA (hnRNA), promoter-associated RNAs (pRNAs), non-coding RNA element which regulates ribosomal RNA transcription by interacting with TIP5 (NoRC RNA), a ribozyme, anti-microRNA (antimiR), an aptamer, or an exon skipping oligonucleotide. In some embodiments, the engineered polynucleotide comprises a completely synthetic miRNA. A completely synthetic miRNA is one that is not derived or based upon an ncRNA. Instead, a completely synthetic miRNA may be based upon an analysis of multiple potential target sequences or may be based upon isolated natural non-coding sequences that are not ncRNAs. In some embodiments, the polynucleotide is selected from the group consisting of a siRNA, a miRNA, a miRNA mimic, an antisense oligonucleotide (ASO), an mRNA, and a guide RNA. The polynucleotide may be a siRNA. In some embodiments, the polynucleotide is a miRNA. In some embodiments, the polynucleotide is a miRNA mimic.

In some embodiments, the polynucleotide is an ASO. In some embodiments, the ASO is an DMPK ASO. In some embodiments, the ASO is a CAPN3 ASO. The ASO may be a DUX4-targeted ASO. DUX4-targeted ASOs are known in the art. See, e.g., WO 2021/203043 and U.S. Provisional Patent Application No. 63/221,568, each of which is incorporated herein by reference in its entirety. Additional non-limiting examples of DUX4-targeted ASOs are provided in Table 1, infra. In some embodiments, the DUX4-targeted ASO is selected from the group consisting of ASDX2, ASDX4, ASDX23, ASDX26, and ASDX32. In some embodiments, the DUX4-targeted ASO is ASDX2. In some embodiments, the DUX4-targeted ASO is ASDX4. In some embodiments, the DUX4-targeted ASO is ASDX23. In some embodiments, the DUX4-targeted ASO is ASDX26. In some embodiments, the DUX4-targeted ASO is ASDX32.

TABLE 1
Sequences of DUX4-targeted Antisense oligonucleotides

| Oligo | Sequence (5′-3′)² |
|---|---|
| ASDX1 | {G}*{C}*c*a*t*c*g*c*g*g*g*t*a*g*{C}*{C} |
| ASDX2 | Amino C6-{G}*{C}*c*a*t*c*g*c*g*g*g*t*a*g*{C}*{C} |
| ASDX3 | Amino C6-{T}*{G}*t*c*g*g*g*a*g*g*g*c*c*a*{T}*{C} |
| ASDX4 | Amino C6-{T}{G}t*c*g*g*g*a*g*g*g*c*c*a{T}{C} |
| ASDX5 | Amino C6-{T}mp{G}t*c*g*g*g*a*g*g*g*c*c*a{T}mp{C} |
| ASDX6 | Amino C6-{T}*{G}*t*c*g*g*g*a*g*g*g*c*c*a*{T}*{C} |
| ASDX7 | {G}*{C}*c*a*t*c*g*c*g*g*g*t*a*g*{C}*{C} |
| ASDX8 | {T}*{C}*c*a*a*a*c*g*a*g*t*c*t*c*{C}*{G} |
| ASDX9 | Amino C6-{T}*{C}*c*a*a*a*c*g*a*g*t*c*t*c*{C}*{G} |
| ASDX10 | {G}*{A}*t*t*c*t*g*a*a*a*c*c*a*g*{A}*{T} |
| ASDX11 | Amino C6-{G}*{A}*t*t*c*t*g*a*a*a*c*c*a*g*{A}*{T} |
| ASDX12 | {G}*{C}*g*g*g*c*g*c*c*c*t*g*c*c*{A}*{C} |
| ASDX13 | Amino C6-{G}*{C}*g*g*g*c*g*c*c*c*t*g*c*c*{A}*{C} |
| ASDX14 | {T}*{C}*a*t*c*c*a*g*c*a*g*c*a*g*{G}*{C} |
| ASDX15 | Amino C6-{T}*{C}*a*t*c*c*a*g*c*a*g*c*a*g*{G}*{C} |
| ASDX16 | {T}*{A}*g*c*c*a*g*c*c*a*g*g*t*g*{T}*{T} |
| ASDX17 | Amino C6-{T}*{A}*g*c*c*a*g*c*c*a*g*g*t*g*{T}*{T} |
| ASDX18 | {C}*{A}*g*c*g*t*c*g*g*a*a*g*g*t*{G}*{G} |
| ASDX19 | Amino C6-{C}*{A}*g*c*g*t*c*g*g*a*a*g*g*t*{G}*{G} |
| ASDX20 | {T}*{A}*g*a*c*a*g*c*g*t*c*g*g*a*{A}*{G} |
| ASDX21 | Amino C6-{T}*{A}*g*a*c*a*g*c*g*t*c*g*g*a*{A}*{G} |
| ASDX22 | {A}*{T}*a*g*g*a*t*c*c*a*c*a*g*g*{G}*{A} |
| ASDX23 | Amino C6-{A}*{T}*a*g*g*a*t*c*c*a*c*a*g*g*{G}*{A} |
| ASDX24 | Amino C6-{A}*{T}*a*g*g*a*t*c*c*a*c*a*g*g*{G}*{A} |
| ASDX25 | {T}*{C}*t*a*t*a*g*g*a*t*c*c*a*c*{A}*{G} |
| ASDX26 | Amino C6-{T}*{C}*t*a*t*a*g*g*a*t*c*c*a*c*{A}*{G} |
| ASDX27 | {G}*{C}*a*c*t*a*a*t*c*a*t*c*c*a*{G}*{G} |
| ASDX28 | Amino C6-{G)*{C}*a*c*t*a*a*t*c*a*t*c*c*a*{G}*{G} |
| ASDX29 | {C}*{A}*{G}*c*g*t*c*g*g*a*a*g*{G}*{T}*{G} |
| ASDX30 | Amino C6-{C}*{A}*{G}*c*g*t*c*g*g*a*a*g*{G}*{T}*{G} |
| ASDX31 | (C)*(C)*(A)*(G)*c*g*t*c*g*g*a*a*g*(G)*(T)*(G)*(T) |
| ASDX32 | Amino C6-(C)*(C)*(A)*(G)*c*g*t*c*g*g*a*a*g*(G)*(T)*(G)*(T) |
| ASDX33 | {C}*{C}*t*{A}*g*a*c*a*g*c*g*t*c*g*{G}*{A}*a*g*{G}*{T} |
| ASDX34 | {A}*{T}*{A}*g*g*a*t*c*c*a*c*a*g*g*g*{A}*{G}*{G} |
| ASDX35 | {C}*{G}*{G}*c*t*c*t*g*g*g*a*t*c*c*c*{C}*{G}*{G} |
| ASDX36 | {G}*{G}*{G}*g*c*g*g*a*g*a*c*a*c*g*{C}*{C}*{C} |
| ASDX37 | {A}*{G}*{A}*a*g*g*c*a*g*g*a*a*t*c*c*{C}*{A}*{G} |
| ASDX38 | {G}*{C}*{A}*g*g*a*a*t*c*c*c*g*g*g*c*{C}*{G}*{G} |
| ASDX39 | {G}*{G}*{A}*g*t*c*t*c*t*c*a*c*c*g*g*{G}*{C}*{C} |
| ASDX40 | {A}*{G}*{A}*g*g*c*c*a*g*c*g*a*g*c*t*{C}*{C}*{C} |
| ASDX41 | {G}*{G}*{C}*t*c*t*g*g*g*a*t*c*c*c*c*{G)*{G}*{G} |
| ASDX42 | {C}*{A}*{G}*a*g*a*g*g*c*c*a*g*c*g*a*{G}*{C}*{T} |
| ASDX43 | {G}*{A}*{C}*a*g*c*g*t*c*g*g*a*a*g*g*{T}*{G}*{G} |
| ASDX44 | {G}*{T}*{A}*a*c*t*c*t*a*a*t*c*c*a*g*{G}*{T}*{T} |
| ASDX45 | {A}*{C}*{A}*a*g*g*g*c*a*c*a*g*a*g*a*{G}*{G}*{C} |
| ASDX46 | {G}*{A}*{G}*c*t*c*c*c*t*t*g*c*a*c*g*{T}*{C}*{A} |
| ASDX47 | {C}*{C}*{G}*t*c*c*a*a*c*c*c*c*g*c*{G}*{T}*{C} |
| ASDX48 | {C}*{C}*{T}*a*a*a*g*c*t*c*c*t*c*c*a*{G}*{C}*{A} |
| ASDX49 | {G}*{C}*{G}*a*g*g*c*g*g*c*c*t*c*t*t*{C}*{C}*{G} |
| ASDX50 | {G}*{C}*{C}*t*c*c*a*g*c*t*c*c*c*c*c*{G}*{G}*{G} |
| ASDX51 | {G}*{G}*{T}*g*t*c*g*g*g*a*g*g*g*c*{C}*{A}*{T} |
| ASDX52 | {C}*{T}*{G}*g*g*c*c*a*g*c*c*g*t*t*c*{T}*{C}*{T} |
| ASDX53 | {G}*{G}*{G)*c*c*a*g*c*c*g*t*t*c*t*c*{T}*{G}*{G} |
| ASDX54 | {C}*{A}*a*t*t*t*c*a*g*g*c*t*t*t*{T}*t*{C}*{T} |
| ASDX55 | {T}*{G}*{C}*c*t*a*c*a*g*a*a*g*g*c*t*{T}*{T}*{G} |
| ASDX56 | {A}*{T}*c*t*c*t*g*c*a*c*t*c*a*t*{C}*a*{C}*{A} |
| ASDX57 | {C}*{T}*g*a*t*c*a*c*c*g*a*a*g*t*{T}*c*{T}*{G} |
| ASDX58 | {A}*{T}*a*g*g*a*t*c*c*a*c*a*g*g*{G}*a*{G}*{G} |
| ASDX59 | {C}*{C}*a*g*g*a*g*a*t*g*t*a*a*c*{T}*c*{T}*{A} |
| ASDX60 | {G}*{A}*{A}*a*g*a*g*a*g*g*c*c*a*c*c*{G}*{C}*{C} |
| ASDX61 | {G}*{T}*{A}*g*c*c*a*g*c*c*a*g*g*t*g*{T}*{T}*{C} |
| ASDX62 | {G}*{C}*{C}*c*c*t*c*c*g*t*a*g*c*c*a*{G}*{C}*{C} |
| ASDX63 | {T}*{G}*{C}*t*g*t*c*c*g*a*g*g*g*t*g*{T}*{C}*{G} |
| ASDX64 | {A}*{G}*{G}*g*g*t*g*c*t*t*c*c*a*g*c*{G}*{A}*{G} |
| ASDX65 | {T}*{T}*{C}*t*t*c*c*t*c*g*c*t*g*a*g*{G}*{G}*{G} |
| ASDX66 | {C}*{G}*{G)*t*a*t*t*c*t*t*c*c*t*c*g*{C}*{T}*{G} |
| ASDX67 | {T}*{C}*{C}*t*c*c*a*g*c*a*g*a*g*c*c*{C}*{G}*{G} |
| ASDX68 | {C}*{C}*{T}*g*g*g*c*c*g*g*c*t*c*t*g*{G}*{G}*{A} |
| ASDX69 | {T}*{G}*{C}*t*g*g*t*a*c*c*t*g*g*g*c*{C}*{G}*{G} |
| ASDX70 | {T}*{C}*{T}*a*t*a*g*g*a*t*c*c*a*c*a*{G}*{G}*{G} |

²{N} is a locked nucleic acid (LNA); (C) is a 2′ methoxy ethyl; [N] is a branched nucleic acid (BNA); * is a phosphothionate-modified backbone. (m)p is a methylphosphonate

In some embodiments, the polynucleotide comprises a siRNA, a miRNA, a miRNA mimic, an ASO, or a guide RNA that targets Dystrophin, DUX4, DMPK or CAPN3. In some embodiments, the polynucleotide comprises a siRNA that targets DUX4. In some embodiments, the polynucleotide comprises a miRNA that targets DUX4. In some embodiments, the polynucleotide comprises a miRNA mimic that targets DUX4. In some embodiments, the polynucleotide comprises an ASO that targets DUX4. In some embodiments, the polynucleotide comprises a guide RNA that targets DUX4. In some embodiments, the polynucleotide comprises a siRNA that targets DMPK. In some embodiments, the polynucleotide comprises a miRNA that targets DMPK. In some embodiments, the polynucleotide comprises a miRNA mimic that targets DMPK. In some embodiments, the polynucleotide comprises an ASO that targets DMPK. In some embodiments, the polynucleotide comprises a siRNA that targets CAPN3. In some embodiments, the polynucleotide comprises a miRNA that targets CAPN3. In some embodiments, the polynucleotide comprises a miRNA mimic that targets CAPN3. In some embodiments, the polynucleotide comprises an ASO that targets CAPN3.

In some embodiments, the polynucleotide is a coding RNA. In some embodiments, the polynucleotide is a mRNA. In some embodiments, the polynucleotide is a non-coding RNA. In some embodiments, the polynucleotide is a long non-coding RNA. In some embodiments, the polynucleotide is a guide RNA.

In some embodiments, the polynucleotide comprises one or more artificial nucleotide analogues. In some instances, the artificial nucleotide analogues comprise modifications at one or more of ribose moiety, phosphate moiety, nucleoside moiety, or a combination thereof. In some embodiments, one or more of the artificial nucleotide analogues are resistant toward nucleases such as for example ribonuclease such as RNase, deoxyribonuclease such as DNase, or exonuclease such as 5′-3′ exonuclease and 3′-5′ exonuclease when compared to natural polynucleotides. In some embodiments, artificial nucleotide analogues comprising 2′-O-methyl, 2′-O-methoxyethyl (2′-O-MOE), 2′-O-aminopropyl, 2′-deoxy, T-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), T-O-dimethylaminoethyloxyethyl (2′-O-DMAEOE), or 2′-O—N-methylacetamido (2′-O-NMA) modified, LNA, ENA, PNA, HNA, BNA, morpholino, methylphosphonate nucleotides, thiolphosphonate nucleotides, 2′-fluoro N3-P5′-phosphoramidites, or combinations thereof are resistant toward nucleases such as for example ribonuclease such as RNase, deoxyribonuclease such as DNase, or exonuclease such as 5′-3′ exonuclease and 3′-5′ exonuclease. In some embodiments, 2′-O-methyl modified polynucleotide is nuclease resistant (e.g., RNase, DNase, 5′-3′ exonuclease or 3′-5′ exonuclease resistant). In some embodiments, 2′O-methoxyethyl (2′-O-MOE) modified polynucleotide is nuclease resistant (e.g., RNase, DNase, 5′-3′ exonuclease or 3′-5′ exonuclease resistant). In some embodiments, 2′-O-aminopropyl modified polynucleotide is nuclease resistant (e.g., RNase, DNase, 5′-3′ exonuclease or 3′-5′ exonuclease resistant). In some embodiments, 2′-deoxy modified polynucleotide is nuclease resistant (e.g., RNase, DNase, 5′-3′ exonuclease or 3′-5′ exonuclease resistant). In some embodiments, T-deoxy-2′-fluoro modified polynucleotide is nuclease resistant (e.g., RNase, DNase, 5′-3′ exonuclease or 3′-5′ exonuclease resistant). In some embodiments, 2′-O-aminopropyl (2′-O-AP) modified polynucleotide is nuclease resistant (e.g., RNase, DNase, 5′-3′ exonuclease or 3′-5′ exonuclease resistant). In some embodiments, 2′-O-dimethylaminoethyl (2′-O-DMAOE) modified polynucleotide is nuclease resistant (e.g., RNase, DNase, 5′-3′ exonuclease or 3′-5′ exonuclease resistant). In some embodiments, 2′-O-dimethylaminopropyl (2′-O-DMAP) modified polynucleotide is nuclease resistant (e.g., RNase, DNase, 5′-3′ exonuclease or 3′-5′ exonuclease resistant). In some embodiments, T-O-dimethylaminoethyloxyethyl (2′-O-DMAEOE) modified polynucleotide is nuclease resistant (e.g., RNase, DNase, 5′-3′ exonuclease or 3′-5′ exonuclease resistant). In some embodiments, 2′-O—N-methylacetamido (2′-O-NMA) modified polynucleotide is nuclease resistant (e.g., RNase, DNase, 5′-3′ exonuclease or 3′-5′ exonuclease resistant). In some embodiments, LNA-modified polynucleotide is nuclease resistant (e.g., RNase, DNase, 5′-3′ exonuclease or 3′-5′ exonuclease resistant). In some embodiments, ENA-modified polynucleotide is nuclease resistant (e.g., RNase, DNase, 5′-3′ exonuclease or 3′-5′ exonuclease resistant). In some embodiments, HNA-modified polynucleotide is nuclease resistant (e.g., RNase, DNase, 5′-3′ exonuclease or 3′-5′ exonuclease resistant). Morpholinos may be nuclease resistant (e.g., RNase, DNase, 5′-3′ exonuclease or 3′-5′ exonuclease resistant). In some embodiments, PNA-modified polynucleotide is resistant to nucleases (e.g., RNase, DNase, 5′-3′ exonuclease or 3′-5′ exonuclease resistant). In some embodiments, methylphosphonate nucleotide-modified polynucleotide is nuclease resistant (e.g., RNase, DNase, 5′-3′ exonuclease or 3′-5′ exonuclease resistant). In some embodiments, thiolphosphonate nucleotide-modified polynucleotide is nuclease resistant (e.g., RNase, DNase, 5′-3′ exonuclease or 3′-5′ exonuclease resistant). In some embodiments, polynucleotide comprising 2′-fluoro N3-P5′-phosphoramidites is nuclease resistant (e.g., RNase, DNase, 5′-3′ exonuclease or 3′-5′ exonuclease resistant). In some embodiments, the 5′ conjugates described herein inhibit 5′-3′ exonucleolytic cleavage. In some embodiments, the 3′ conjugates described herein inhibit 3′-5′ exonucleolytic cleavage.

In some embodiments, one or more of the artificial nucleotide analogues described herein have increased binding affinity toward their mRNA target relative to an equivalent natural polynucleotide. In some embodiments, the artificial nucleotide analogue comprises a nucleic acid with a modification at a 2′ hydroxyl group of the ribose moiety. In some embodiments, the modification includes an H, OR, R, halo, SH, SR, NH₂, NHR, NR₂, or CN, wherein R is an alkyl moiety. Exemplary alkyl moieties include, but are not limited to, halogens, sulfurs, thiols, thioethers, thioesters, amines (primary, secondary, or tertiary), amides, ethers, esters, alcohols and oxygen. In some embodiments, the alkyl moiety further comprises a modification. In some embodiments, the modification comprises an azo group, a keto group, an aldehyde group, a carboxyl group, a nitro group, a nitroso, group, a nitrile group, a heterocycle (e.g., imidazole, hydrazino or hydroxylamino) group, an isocyanate or cyanate group, or a sulfur containing group (e.g., sulfoxide, sulfone, sulfide, or disulfide). In some embodiments, the alkyl moiety further comprises a hetero substitution. In some embodiments, the carbon of the heterocyclic group is substituted by a nitrogen, oxygen or sulfur. In some embodiments, the heterocyclic substitution includes but is not limited to, morpholino, imidazole, and pyrrolidino. The one or more of the artificial nucleotide analogues comprising 2′-O-methyl, 2′-O-methoxyethyl (2′-O-MOE), 2′-O-aminopropyl, 2′-deoxy, T-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), T-O-dimethylaminoethyloxyethyl (2′-O-DMAEOE), or 2′-O—N-methylacetamido (2′-O-NMA) modified, LNA, ENA, PNA, HNA, morpholino, methylphosphonate nucleotides, thiolphosphonate nucleotides, or 2′-fluoro N3-P5′-phosphoramidites can have increased binding affinity toward their mRNA target relative to an equivalent natural polynucleotide. In some embodiments, 2′-O-methyl modified polynucleotide has increased binding affinity toward their mRNA target relative to an equivalent natural polynucleotide. In some embodiments, 2′-O-methoxyethyl (2′-O-MOE) modified polynucleotide has increased binding affinity toward their mRNA target relative to an equivalent natural polynucleotide. In some embodiments, 2′-O-aminopropyl modified polynucleotide has increased binding affinity toward their mRNA target relative to an equivalent natural polynucleotide. In some embodiments, 2′-deoxy modified polynucleotide has increased binding affinity toward their mRNA target relative to an equivalent natural polynucleotide. In some embodiments, T-deoxy-2′-fluoro modified polynucleotide has increased binding affinity toward their mRNA target relative to an equivalent natural polynucleotide. In some embodiments, 2′-O-aminopropyl (2′-O-AP) modified polynucleotide has increased binding affinity toward their mRNA target relative to an equivalent natural polynucleotide. In some embodiments, 2′-O-dimethylaminoethyl (2′-O-DMAOE) modified polynucleotide has increased binding affinity toward their mRNA target relative to an equivalent natural polynucleotide. In some embodiments, 2′-O-dimethylaminopropyl (2′-O-DMAP) modified polynucleotide has increased binding affinity toward their mRNA target relative to an equivalent natural polynucleotide. In some embodiments, T-O-dimethylaminoethyloxyethyl (2′-O-DMAEOE) modified polynucleotide has increased binding affinity toward their mRNA target relative to an equivalent natural polynucleotide. In some embodiments, 2′-O—N-methylacetamido (2′-O-NMA) modified polynucleotide has increased binding affinity toward their mRNA target relative to an equivalent natural polynucleotide. In some embodiments, LNA-modified polynucleotide has increased binding affinity toward their mRNA target relative to an equivalent natural polynucleotide. In some embodiments, ENA-modified polynucleotide has increased binding affinity toward their mRNA target relative to an equivalent natural polynucleotide. In some embodiments, PNA-modified polynucleotide has increased binding affinity toward their mRNA target relative to an equivalent natural polynucleotide. In some embodiments, HNA-modified polynucleotide has increased binding affinity toward their mRNA target relative to an equivalent natural polynucleotide. In some embodiments, morpholino-modified polynucleotide has increased binding affinity toward their mRNA target relative to an equivalent natural polynucleotide. In some embodiments, methylphosphonate nucleotide-modified polynucleotide has increased binding affinity toward their mRNA target relative to an equivalent natural polynucleotide. In some embodiments, thiolphosphonate nucleotide-modified polynucleotide has increased binding affinity toward their mRNA target relative to an equivalent natural polynucleotide. In some embodiments, polynucleotide comprising 2′-fluoro N3-P5′-phosphoramidites has increased binding affinity toward their mRNA target relative to an equivalent natural polynucleotide. In some embodiments, the increased affinity is illustrated with a lower Kd, a higher melt temperature (Tm), or a combination thereof.

In some embodiments, the artificial nucleotide analogues include 2′-O-methyl, 2′-O-methoxyethyl (2′-O-MOE), 2′-O-aminopropyl, 2′-deoxy, T-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), T-O-dimethylaminoethyloxyethyl (2′-O-DMAEOE), or 2′-O—N-methylacetamido (2′-O-NMA) modified, LNA, ENA, PNA, HNA, morpholino, methylphosphonate nucleotides, thiolphosphonate nucleotides, 2′-fluoro N3-P5′-phosphoramidites, or a combination thereof.

In some embodiments, the artificial nucleotide analogue comprises a modified base such as, but not limited to, 5-propynyluridine, 5-propynylcytidine, 6-methyladenine, 6-methylguanine, N,N,-dimethyladenine, 2-propyladenine, 2propylguanine, 2-aminoadenine, 1-methylinosine, 3-methyluridine, 5-methylcytidine, 5-methyluridine and other nucleotides having a modification at the 5 position, 5-(2-amino) propyl uridine, 5-halocytidine, 5-halouridine, 4-acetylcytidine, 1-methyladenosine, 2-methyladenosine, 3-methylcytidine, 6-methyluridine, 2-methylguanosine, 7-methylguanosine, 2, 2-dimethylguanosine, 5-methylaminoethyluridine, 5-methyloxyuridine, deazanucleotides (such as 7-deaza-adenosine, 6-azouridine, 6-azocytidine, or 6-azothymidine), 5-methyl-2-thiouridine, other thio bases (such as 2-thiouridine, 4-thiouridine, and 2-thiocytidine), dihydrouridine, pseudouridine, queuosine, archaeosine, naphthyl and substituted naphthyl groups, any O- and N-alkylated purines and pyrimidines (such as N6-methyladenosine, 5-methylcarbonylmethyluridine, uridine 5-oxyacetic acid, pyridine-4-one, or pyridine-2-one), phenyl and modified phenyl groups such as aminophenol or 2,4,6-trimethoxy benzene, modified cytosines that act as G-clamp nucleotides, 8-substituted adenines and guanines, 5-substituted uracils and thymines, azapyrimidines, carboxyhydroxyalkyl nucleotides, carboxyalkylaminoalkyl nucleotides, and alkylcarbonylalkylated nucleotides. Modified nucleotides also include those nucleotides that are modified with respect to the sugar moiety, as well as nucleotides having sugars or analogs thereof that are not ribosyl. For example, the sugar moieties, In some embodiments are or are based on, mannoses, arabinoses, glucopyranoses, galactopyranoses, 4′-thioribose, and other sugars, heterocycles, or carbocycles. The term nucleotide also includes what are known in the art as universal bases. By way of example, universal bases include but are not limited to 3-nitropyrrole, 5-nitroindole, or nebularine.

In some embodiments, the polynucleotide comprises one or more phosphorothioate internucleotide linkages. In some embodiments, the polynucleotide comprises 2′-5′ internucleotide linkages. In some embodiments, the 2′-5′ internucleotide linkage(s) is at the 3′-end, the 5′-end, or both of the 3′- and 5′-ends of one or both sequence strands. In some embodiments, the 2′-5′ internucleotide linkage(s) is present at various other positions within one or both sequence strands. In some embodiments, the polynucleotide comprises a terminal cap molecule at the 3′-end, the 5′-end, or both of the 3′- and 5′-ends.

In some embodiments, the targeting molecule and the polynucleotide combined to provide a synergistic therapeutic or biological effect.

In some embodiments, the polynucleotide is conjugated directly to the targeting molecule. The polynucleotide may be conjugated to the targeting molecule via a linker. Suitable linkers for conjugating polynucleotides to targeting molecules are known in the art. See, e.g., WO 2017/173408, incorporated herein by reference in its entirety. In some embodiments, the linker is a hydrophobic linker. The linker may be a peptide linker. In some embodiments, the linker is a chemical linker. The chemical linker may be a polymeric linker. In some embodiments, the chemical linker is linear. In some embodiments, the chemical linker is cyclic.

In some embodiments, the polymeric linker comprises PEG, a sugar, a fatty acid, a phosphate, a pyrophosphate or a polysarcosine. In some embodiments, the polymeric linker comprises PEG. In some embodiments, the polymeric linker comprises a sugar. In some embodiments, the polymeric linker comprises a fatty acid. In some embodiments, the polymeric linker comprises a phosphate. In some embodiments, the polymeric linker comprises a pyrophosphate. In some embodiments, the polymeric linker comprises a polysarcosine. The linker may be a high molecular weight PEG linker. In some embodiments, the high molecular weight PEG linker comprises between 1,000 and 5,000 PEG monomers (i.e. is between PEG1k and PEG5k). In some embodiments, the high molecular weight PEG linker is PEG1k. In some embodiments, the high molecular weight PEG linker is PEG1.5k. In some embodiments, the high molecular weight PEG linker is PEG2k. In some embodiments, the high molecular weight PEG linker is PEG3k. In some embodiments, the high molecular weight PEG linker is PEG4k. In some embodiments, the high molecular weight PEG linker is PEG5k.

In some embodiments, the linker is a low molecular weight PEG linker. In some embodiments, the low molecular weight PEG linker comprises between 4 and 100 PEG monomers (i.e. is between PEG4 and PEG100). In some embodiments, the low molecular PEG linker is between PEG12 and PEG48. In some embodiments, the low molecular PEG linker is between PEG12 and PEG24. In some embodiments, the low molecular PEG linker is between PEG12 and PEG18. In some embodiments, the low molecular PEG linker is between PEG6 and PEG18. In some embodiments, the low molecular weight PEG linker is PEG4. In some embodiments, the low molecular weight PEG linker is PEG5. In some embodiments, the low molecular weight PEG linker is PEG6. In some embodiments, the low molecular weight PEG linker is PEG7. In some embodiments, the low molecular weight PEG linker is PEG8. In some embodiments, the low molecular weight PEG linker is PEG9. In some embodiments, the low molecular weight PEG linker is PEG10. In some embodiments, the low molecular weight PEG linker is PEG11. In some embodiments, the low molecular weight PEG linker is PEG12. In some embodiments, the low molecular weight PEG linker is PEG13. In some embodiments, the low molecular weight PEG linker is PEG14. In some embodiments, the low molecular weight PEG linker is PEG15. In some embodiments, the low molecular weight PEG linker is PEG16. In some embodiments, the low molecular weight PEG linker is PEG17. In some embodiments, the low molecular weight PEG linker is PEG18. In some embodiments, the low molecular weight PEG linker is PEG19. In some embodiments, the low molecular weight PEG linker is PEG20. In some embodiments, the low molecular weight PEG linker is PEG21. In some embodiments, the low molecular weight PEG linker is PEG22. In some embodiments, the low molecular weight PEG linker is PEG23. In some embodiments, the low molecular weight PEG linker is PEG24. In some embodiments, the low molecular weight PEG linker is PEG25. In some embodiments, the low molecular weight PEG linker is PEG26. In some embodiments, the low molecular weight PEG linker is PEG27. In some embodiments, the low molecular weight PEG linker is PEG28. In some embodiments, the low molecular weight PEG linker is PEG29. In some embodiments, the low molecular weight PEG linker is PEG30. In some embodiments, the low molecular weight PEG linker is PEG31. In some embodiments, the low molecular weight PEG linker is PEG32. In some embodiments, the low molecular weight PEG linker is PEG33. In some embodiments, the low molecular weight PEG linker is PEG34. In some embodiments, the low molecular weight PEG linker is PEG35. In some embodiments, the low molecular weight PEG linker is PEG36. In some embodiments, the low molecular weight PEG linker is PEG37. In some embodiments, the low molecular weight PEG linker is PEG38. In some embodiments, the low molecular weight PEG linker is PEG39. In some embodiments, the low molecular weight PEG linker is PEG40. In some embodiments, the low molecular weight PEG linker is PEG41. In some embodiments, the low molecular weight PEG linker is PEG42. In some embodiments, the low molecular weight PEG linker is PEG43. In some embodiments, the low molecular weight PEG linker is PEG44. In some embodiments, the low molecular weight PEG linker is PEG45. In some embodiments, the low molecular weight PEG linker is PEG46. In some embodiments, the low molecular weight PEG linker is PEG47. In some embodiments, the low molecular weight PEG linker is PEG48. In some embodiments, the low molecular weight PEG linker is PEG49. In some embodiments, the low molecular weight PEG linker is PEG50. In some embodiments, the low molecular weight PEG linker is PEG51. In some embodiments, the low molecular weight PEG linker is PEG52. In some embodiments, the low molecular weight PEG linker is PEG53. In some embodiments, the low molecular weight PEG linker is PEG54. In some embodiments, the low molecular weight PEG linker is PEG55. In some embodiments, the low molecular weight PEG linker is PEG56. In some embodiments, the low molecular weight PEG linker is PEG57. In some embodiments, the low molecular weight PEG linker is PEG58. In some embodiments, the low molecular weight PEG linker is PEG59. In some embodiments, the low molecular weight PEG linker is PEG60. In some embodiments, the low molecular weight PEG linker is PEG61. In some embodiments, the low molecular weight PEG linker is PEG62. In some embodiments, the low molecular weight PEG linker is PEG63. In some embodiments, the low molecular weight PEG linker is PEG64. In some embodiments, the low molecular weight PEG linker is PEG65. In some embodiments, the low molecular weight PEG linker is PEG66. In some embodiments, the low molecular weight PEG linker is PEG67. In some embodiments, the low molecular weight PEG linker is PEG68. In some embodiments, the low molecular weight PEG linker is PEG69. In some embodiments, the low molecular weight PEG linker is PEG70. In some embodiments, the low molecular weight PEG linker is PEG71. In some embodiments, the low molecular weight PEG linker is PEG72. In some embodiments, the low molecular weight PEG linker is PEG73. In some embodiments, the low molecular weight PEG linker is PEG74. In some embodiments, the low molecular weight PEG linker is PEG75. In some embodiments, the low molecular weight PEG linker is PEG76. In some embodiments, the low molecular weight PEG linker is PEG77. In some embodiments, the low molecular weight PEG linker is PEG78. In some embodiments, the low molecular weight PEG linker is PEG79. In some embodiments, the low molecular weight PEG linker is PEG80. In some embodiments, the low molecular weight PEG linker is PEG81. In some embodiments, the low molecular weight PEG linker is PEG82. In some embodiments, the low molecular weight PEG linker is PEG83. In some embodiments, the low molecular weight PEG linker is PEG84. In some embodiments, the low molecular weight PEG linker is PEG85. In some embodiments, the low molecular weight PEG linker is PEG86. In some embodiments, the low molecular weight PEG linker is PEG87. In some embodiments, the low molecular weight PEG linker is PEG88. In some embodiments, the low molecular weight PEG linker is PEG89. In some embodiments, the low molecular weight PEG linker is PEG90. In some embodiments, the low molecular weight PEG linker is PEG91. In some embodiments, the low molecular weight PEG linker is PEG92. In some embodiments, the low molecular weight PEG linker is PEG93. In some embodiments, the low molecular weight PEG linker is PEG94. In some embodiments, the low molecular weight PEG linker is PEG95. In some embodiments, the low molecular weight PEG linker is PEG96. In some embodiments, the low molecular weight PEG linker is PEG97. In some embodiments, the low molecular weight PEG linker is PEG98. In some embodiments, the low molecular weight PEG linker is PEG99. In some embodiments, the low molecular weight PEG linker is PEG100.

In some embodiments, the linker is non-cleavable. In some embodiments, the linker is cleavable. The linker may be cleavable in vivo. In some embodiments, the cleavable linker is selected from the group consisting of a disulfide linker, a self-immolative peptide polymer hybrid, and a sulfatase-promoted arylsulfate linker. In some embodiments, the cleavable linker is a disulfide linker. The cleavable linker may be a self-immolative peptide polymer hybrid. In some embodiments, the cleavable linker is a sulfatase-promoted arylsulfate linker. In some embodiments, the self-immolative peptide polymer hybrid comprises glucuronic acid, para-amino-benzoyloxy (PAB), 7-amino-3-hydroxyethyl-coumarin (7-AHC), or Fe(II)-reactive 1,2,4-trioxolane scaffold (TRX). In some embodiments, the self-immolative peptide polymer hybrid comprises glucuronic acid. In some embodiments, the self-immolative peptide polymer hybrid comprises para-amino-benzoyloxy (PAB). In some embodiments, the self-immolative peptide polymer hybrid comprises 7-amino-3-hydroxyethyl-coumarin (7-AHC). In some embodiments, the self-immolative peptide polymer hybrid comprises Fe(II)-reactive 1,2,4-trioxolane scaffold (TRX).

In some embodiments, the cleavable linker is cleaved through reduction, hydrolysis, proteolysis, photo cleavage, chemical cleavage, enzymatic cleavage, or bio-orthogonal-cleavage. In some embodiments, the cleavable linker is cleaved through reduction. In some embodiments, the cleavable linker is cleaved through hydrolysis. In some embodiments, the cleavable linker is cleaved through proteolysis. In some embodiments, the cleavable linker is cleaved through photo cleavage. In some embodiments, the cleavable linker is cleaved through chemical cleavage. The chemical cleavage may be by Fe II mediated R elimination of TRX. In some embodiments, the cleavable linker is cleaved through enzymatic cleavage. The enzymatic cleavage may be by non-proteolytic sulfatase, β-galactosidase/glucuronidase or pyrophosphatase. In some embodiments, the enzymatic cleavage is by non-proteolytic sulfatase. In some embodiments, the enzymatic cleavage is by β-galactosidase/glucuronidase. In some embodiments, the enzymatic cleavage is by pyrophosphatase. In some embodiments, the cleavable linker is cleaved through bio-orthogonal-cleavage. The bio-orthogonal cleavage may be by Cu I-BTTAA or free copper ion mediated cleavage. In some embodiments, the linker is an acid cleavable linker.

In some embodiments, the linker includes a C₁-C₆ alkyl group (e.g., a C₅, C₄, C₃, C₂, or C₁ alkyl group). In some embodiments, the linker includes homobifunctional cross linkers, heterobifunctional cross linkers, and the like. In some embodiments, the liker is a traceless linker (or a zero-length linker). In some embodiments, the linker is a non-polymeric linker. In some embodiments, the linker is a non-peptide linker or a linker that does not contain an amino acid residue.

In some embodiments, the linker comprises a homobifunctional linker. Exemplary homobifunctional linkers include, but are not limited to, Lomant's reagent dithiobis (succinimidylpropionate) DSP, 3,3′-dithiobis(sulfosuccinimidyl proprionate (DTSSP), disuccinimidyl suberate (DSS), bis(sulfosuccinimidyl)suberate (BS), disuccinimidyl tartrate (DST), disulfosuccinimidyl tartrate (sulfo DST), ethylene glycobis(succinimidylsuccinate) (EGS), disuccinimidyl glutarate (DSG), N,N′-disuccinimidyl carbonate (DSC), dimethyl adipimidate (DMA), dimethyl pimelimidate (DMP), dimethyl suberimidate (DMS), dimethyl-3,3′-dithiobispropionimidate (DTBP), 1,4-di-3′-(2′-pyridyldithio)propionamido)butane (DPDPB), bismaleimidohexane (BMH), aryl halide-containing compound (DFDNB), such as e.g. 1,5-difluoro-2,4-dinitrobenzene or 1,3-difluoro-4,6-dinitrobenzene, 4,4′-difluoro-3,3′-dinitrophenylsulfone (DFDNPS), bis-[2-(4-azidosalicylamido)ethyl]disulfide (BASED), formaldehyde, glutaraldehyde, 1,4-butanediol diglycidyl ether, adipic acid dihydrazide, carbohydrazide, o-toluidine, 3,3′-dimethylbenzidine, benzidine, α,α′-ρ-diaminodiphenyl, diiodo-p-xylene sulfonic acid, N,N′-ethylene-bis(iodoacetamide), or N,N′-hexamethylene-bis(iodoacetamide).

In some embodiments, the linker comprises a heterobifunctional linker. Exemplary heterobifunctional linker include, but are not limited to, amine-reactive and sulfhydryl cross-linkers such as N-succinimidyl 3-(2-pyridyldithio)propionate (sPDP), long-chain N-succinimidyl 3-(2-pyridyldithio)propionate (LC-sPDP), water-soluble-long-chain N-succinimidyl 3-(2-pyridyldithio) propionate (sulfo-LC-sPDP), succinimidyloxycarbonyl-a-methyl-a-(2-pyridyldithio)toluene (sMPT), sulfosuccinimidyl-6-[a-methyl-a-(2-pyridyldithio)toluamido]hexanoate (sulfo-LC-sMPT), succinimidyl-4-(N-maleimidomethyl)cyclohexane-1-carboxylate (sMCC), sulfosuccinimidyl-4-(N-maleimidomethyl)cyclohexane-1-carboxylate (sulfo-sMCC), m-maleimidobenzoyl-N-hydroxysuccinimide ester (MBs), m-maleimidobenzoyl-N-hydroxysulfosuccinimide ester (sulfo-MBs), N-succinimidyl(4-iodoacteyl)aminobenzoate (sIAB), sulfosuccinimidyl(4-iodoacteyl)aminobenzoate (sulfo-sIAB), succinimidyl-4-(p-maleimidophenyl)butyrate (sMPB), sulfosuccinimidyl-4-(p-maleimidophenyl)butyrate (sulfo-sMPB), N-(y-maleimidobutyryloxy)succinimide ester (GMBs), N-(γ-maleimidobutyryloxy)sulfosuccinimide ester (sulfo-GMBs), succinimidyl 6-((iodoacetyl)amino)hexanoate (sIAX), succinimidyl 6-[6-(((iodoacetyl)amino)hexanoyl)amino]hexanoate (sIAXX), succinimidyl 4-(((iodoacetyl)amino)methyl)cyclohexane-1-carboxylate (sIAC), succinimidyl 6-((((4-iodoacetyl)amino)methyl)cyclohexane-1-carbonyl)amino) hexanoate (sIACX), p-nitrophenyl iodoacetate (NPIA), carbonyl-reactive and sulfhydryl-reactive cross-linkers such as 4-(4-N-maleimidophenyl)butyric acid hydrazide (MPBH), 4-(N-maleimidomethyl)cyclohexane-1-carboxyl-hydrazide-8 (M2C2H), 3-(2-pyridyldithio)propionyl hydrazide (PDPH), amine-reactive and photoreactive cross-linkers such as N-hydroxysuccinimidyl-4-azidosalicylic acid (NHs-AsA), N-hydroxysulfosuccinimidyl-4-azidosalicylic acid (sulfo-NHs-AsA), sulfosuccinimidyl-(4-azidosalicylamido)hexanoate (sulfo-NHs-LC-AsA), sulfosuccinimidyl-2-(p-azidosalicylamido)ethyl-1,3′-dithiopropionate (sAsD), N-hydroxysuccinimidyl-4-azidobenzoate (HsAB), N-hydroxysulfosuccinimidyl-4-azidobenzoate (sulfo-HsAB), N-succinimidyl-6-(4′-azido-2′-nitrophenylamino)hexanoate (sANPAH), sulfosuccinimidyl-6-(4′-azido-2′-nitrophenylamino)hexanoate (sulfo-sANPAH), N-5-azido-2-nitrobenzoyloxysuccinimide (ANB-NOs), sulfosuccinimidyl-2-(m-azido-o-nitrobenzamido)-ethyl-1,3′-dithiopropionate (sAND), N-succinimidyl-4(4-azidophenyl) 1,3′-dithiopropionate (sADP), N-sulfosuccinimidyl(4-azidophenyl)-1,3′-dithiopropionate (sulfo-sADP), sulfosuccinimidyl 4-(p-azidophenyl)butyrate (sulfo-sAPB), sulfosuccinimidyl 2-(7-azido-4-methylcoumarin-3-acetamide)ethyl-1,3′-dithiopropionate (sAED), sulfosuccinimidyl 7-azido-4-methylcoumain-3-acetate (sulfo-sAMCA), p-nitrophenyl diazopyruvate (pNPDP), p-nitrophenyl-2-diazo-3,3,3-trifluoropropionate (PNP-DTP), sulfhydryl-reactive and photoreactive cross-linkers such as 1-(p-Azidosalicylamido)-4-(iodoacetamido)butane (AsIB), N-[4-(p-azidosalicylamido)butyl]-3′-(2′-pyridyldithio)propionamide (APDP), benzophenone-4-iodoacetamide, benzophenone-4-maleimide carbonyl-reactive and photoreactive cross-linkers such as p-azidobenzoyl hydrazide (ABH), carboxylate-reactive and photoreactive cross-linkers such as 4-(p-azidosalicylamido)butylamine (AsBA), and arginine-reactive and photoreactive cross-linkers such as p-azidophenyl glyoxal (APG).

In some embodiments, the linker comprises a reactive functional group. In some embodiments, the reactive functional group comprises a nucleophilic group that is reactive to an electrophilic group present on a binding moiety. Exemplary electrophilic groups include carbonyl groups—such as aldehyde, ketone, carboxylic acid, ester, amide, enone, acyl halide or acid anhydride. In some embodiments, the reactive functional group is aldehyde. Exemplary nucleophilic groups include hydrazide, oxime, amino, hydrazine, thiosemicarbazone, hydrazine carboxylate, and arylhydrazide.

In some embodiments, the linker comprises a maleimide group. In some embodiments, the maleimide group is also referred to as a maleimide spacer. In some embodiments, the maleimide group further encompasses a caproic acid, forming maleimidocaproyl (me). In some embodiments, the linker comprises maleimidocaproyl (me). In some embodiments, the linker is maleimidocaproyl (me). In other instances, the maleimide group comprises a maleimidomethyl group, such as succinimidyl-4-(N-maleimidomethyl)cyclohexane-1-carboxylate (sMCC) or sulfosuccinimidyl-4-(N-maleimidomethyl)cyclohexane-1-carboxylate (sulfo-sMCC) described above.

In some embodiments, the maleimide group is a self-stabilizing maleimide. In some embodiments, the self-stabilizing maleimide utilizes diaminopropionic acid (DPR) to incorporate a basic amino group adjacent to the maleimide to provide intramolecular catalysis of thiosuccinimide ring hydrolysis, thereby eliminating maleimide from undergoing an elimination reaction through a retro-Michael reaction. In some embodiments, the self-stabilizing maleimide is a maleimide group described in Lyon, et al, “Self-hydrolyzing maleimides improve the stability and pharmacological properties of antibody-drug conjugates,” Nat. Biotechnol. 32(10): 1059-1062 (2014). In some embodiments, the linker comprises a self-stabilizing maleimide. In some embodiments, the linker is a self-stabilizing maleimide.

In some embodiments, the linker comprises a peptide moiety. In some embodiments, the peptide moiety comprises at least 2, 3, 4, 5, 6, 7, 8, or more amino acid residues. In some embodiments, the peptide moiety is a cleavable peptide moiety (e.g., either enzymatically or chemically). In some embodiments, the peptide moiety is a non-cleavable peptide moiety. In some embodiments, the peptide moiety comprises Val-Cit (valine-citrulline), Gly-Gly-Phe-Gly, Phe-Lys, Val-Lys, Gly-Phe-Lys, Phe-Phe-Lys, Ala-Lys, Val-Arg, Phe-Cit, Phe-Arg, Leu-Cit, Ile-Cit, Tφ-Cit, Phe-Ala, Ala-Leu-Ala-Leu, or Gly-Phe-Leu-Gly. In some embodiments, the linker comprises a peptide moiety such as: Val-Cit (valine-citrulline), Gly-Gly-Phe-Gly, Phe-Lys, Val-Lys, Gly-Phe-Lys, Phe-Phe-Lys, Ala-Lys, Val-Arg, Phe-Cit, Phe-Arg, Leu-Cit, Ile-Cit, Trp-Cit, Phe-Ala, Ala-Leu-Ala-Leu, or Gly-Phe-Leu-Gly. In some embodiments, the linker comprises Val-Cit. In some embodiments, the linker is Val-Cit.

In some embodiments, the linker comprises a benzoic acid group, or its derivatives thereof. In some embodiments, the benzoic acid group or its derivatives thereof comprise paraaminobenzoic acid (PABA). In some embodiments, the benzoic acid group or its derivatives thereof comprise gamma-aminobutyric acid (GABA).

In some embodiments, the linker comprises one or more of a maleimide group, a peptide moiety, and/or a benzoic acid group, in any combination. In some embodiments, the linker comprises a combination of a maleimide group, a peptide moiety, and/or a benzoic acid group. In some embodiments, the maleimide group is maleimidocaproyl (me). In some embodiments, the peptide group is val-cit. In some embodiments, the benzoic acid group is PABA. In some embodiments, the linker comprises a mc-val-cit group. In some embodiments, the linker comprises a val-cit-PABA group. In additional cases, the linker comprises a mc-val-cit-PABA group.

In some embodiments, the linker is a self-immolative linker or a self-elimination linker. In some embodiments, the linker is a self-immolative linker. In other cases, the linker is a self-elimination linker (e.g., a cyclization self-elimination linker). In some embodiments, the linker comprises a linker described in U.S. Pat. No. 9,089,614 or PCT Publication No. WO2015038426, each of which is incorporated herein by reference in its entirety.

In some embodiments, the linker is a dendritic type linker. In some embodiments, the dendritic type linker comprises a branching, multifunctional linker moiety. In some embodiments, the dendritic type linker is used to increase the molar ratio of polynucleotide B to the binding moiety A. In some embodiments, the dendritic type linker comprises PAMAM dendrimers.

In some embodiments, the linker is a traceless linker or a linker in which after cleavage does not leave behind a linker moiety (e.g., an atom or a linker group) to a polynucleotide or a targeting molecule. Exemplary traceless linkers include, but are not limited to, germanium linkers, silicium linkers, sulfur linkers, selenium linkers, nitrogen linkers, phosphorus linkers, boron linkers, chromium linkers, or phenylhydrazide linker. In some embodiments, the linker is a traceless aryl-triazene linker as described in Hejesen, et al., “A traceless aryl-triazene linker for DNA-directed chemistry,” Org Biomol Chem 11(15): 2493-2497 (2013). In some embodiments, the linker is a traceless linker described in Blaney, et al., ‘Traceless solid-phase organic synthesis,” Chem. Rev. 102: 2607-2024 (2002). In some embodiments, a linker is a traceless linker as described in U.S. Pat. No. 6,821,783, incorporated herein by reference in its entirety.

In some embodiments, the linker comprises a functional group that exerts steric hinderance at the site of bonding between the linker and a conjugating moiety (e.g., a polynucleotide or a targeting molecule disclosed herein). In some embodiments, the steric hinderance is a steric hindrance around a disulfide bond. Exemplary linkers that exhibit steric hinderance comprises a heterobifunctional linker, such as a heterobifunctional linker described above. In some embodiments, a linker that exhibits steric hinderance comprises SMCC and SPDB.

In some embodiments, the linker is an acid cleavable linker. In some embodiments, the acid cleavable linker comprises a hydrazone linkage, which is susceptible to hydrolytic cleavage. In some embodiments, the acid cleavable linker comprises a thiomaleamic acid linker. In some embodiments, the acid cleavable linker is a thiomaleamic acid linker as described in Castaneda, et al, “Acid-cleavable thiomaleamic acid linker for homogeneous antibody-drug conjugation,” Chem. Commun. 49: 8187-8189 (2013).

In some embodiments, the linker is a linker described in U.S. Pat. Nos. 6,884,869; 7,498,298; 8,288,352; 8,609,105; or 8,697,688; U.S. Patent Publication Nos. 2014/0127239; 2013/028919; 2014/286970; 2013/0309256; 2015/037360; or 2014/0294851; or PCT Publication Nos. WO2015057699; WO2014080251; WO2014197854; WO2014145090; or WO2014177042, each of which is incorporated herein by reference in its entirety.

In some embodiments, the linker is conjugated to a lysine residue, a cysteine residue, a histidine residue, or a non-natural amino acid residue in the targeting molecule. In some embodiments, the linker is conjugated to a lysine residue in the targeting molecule. In some embodiments, the linker is conjugated to a cysteine residue in the targeting molecule. In some embodiments, the linker is conjugated to a histidine residue in the targeting molecule. In some embodiments, the linker is conjugated to a non-natural amino acid residue in the targeting molecule.

In some embodiments, the linker is conjugated to the targeting molecule by a chemical conjugation or an enzymatic conjugation. In some embodiments, the linker is conjugated to the targeting molecule by a chemical conjugation. The chemical conjugation may comprise acylation and click chemistry. In some embodiments, the linker is conjugated to the targeting molecule by an enzymatic conjugation. The enzymatic conjugation may be via a sortase or a transferase enzyme.

In some embodiments, the polynucleotide is conjugated to the targeting molecule by a chemical ligation process. In some embodiments, the polynucleotide is conjugated to the targeting molecule by a native ligation. In some embodiments, the conjugation is as described in: Dawson, et al. “Synthesis of proteins by native chemical ligation,” Science 1994, 266, 776-779; Dawson, et al. “Modulation of Reactivity in Native Chemical Ligation through the Use of Thiol Additives,” J. Am. Chem. Soc. 1997, 119, 4325-4329; Hackeng, et al. “Protein synthesis by native chemical ligation: Expanded scope by using straightforward methodology,” Proc. Natl. Acad. Sci. USA 1999, 96, 10068-10073; or Wu, et al. “Building complex glycopeptides: Development of a cysteine-free native chemical ligation protocol,” Angew. Chem. Int. Ed. 2006, 45, 4116-4125. In some embodiments, the conjugation is as described in U.S. Pat. No. 8,936,910. In some embodiments, the polynucleotide is conjugated to the targeting molecule either site-specifically or non-specifically via native ligation chemistry.

In some embodiments, the polynucleotide is conjugated to the targeting molecule by a site-directed method utilizing a “traceless” coupling technology (Philochem). In some embodiments, the “traceless” coupling technology utilizes an N-terminal 1,2-aminothiol group on the targeting molecule which is then conjugate with a polynucleotide containing an aldehyde group. (see Casi et al., “Site-specific traceless coupling of potent cytotoxic drugs to recombinant antibodies for pharmacodelivery,” JACS 134(13): 5887-5892 (2012))

In some embodiments, the polynucleotide is conjugated to the targeting molecule by a site-directed method utilizing an unnatural amino acid incorporated into the targeting molecule. In some embodiments, the unnatural amino acid comprises p-acetylphenylalanine (pAcPhe). In some embodiments, the keto group of pAcPhe is selectively coupled to an alkoxy-amine derivatived conjugating moiety to form an oxime bond. (see Axup et al., “Synthesis of site-specific antibody-drug conjugates using unnatural amino acids,” PNAS 109(40): 16101-16106 (2012)).

In some embodiments, the polynucleotide is conjugated to the targeting molecule by a site-directed method utilizing an enzyme-catalyzed process. In some embodiments, the site-directed method utilizes SMARTag™ technology (Redwood). In some embodiments, the SMARTag™ technology comprises generation of a formylglycine (FGly) residue from cysteine by formylglycine-generating enzyme (FGE) through an oxidation process under the presence of an aldehyde tag and the subsequent conjugation of FGly to an alkylhydraine-functionalized polynucleotide via hydrazino-Pictet-Spengler (HIPS) ligation. (see Wu et al., “Site-specific chemical modification of recombinant proteins produced in mammalian cells by using the genetically encoded aldehyde tag,” PNAS 106(9): 3000-3005 (2009); Agarwal, et al., “A Pictet-Spengler ligation for protein chemical modification,” PNAS 110(1): 46-51 (2013))

In some embodiments, the enzyme-catalyzed process comprises microbial transglutaminase (mTG). In some embodiments, the polynucleotide is conjugated to the targeting molecule utilizing a microbial transglutaminase catalyzed process. In some embodiments, mTG catalyzes the formation of a covalent bond between the amide side chain of a glutamine within the recognition sequence and a primary amine of a functionalized polynucleotide. In some embodiments, mTG is produced from Streptomyces mobarensis. (see Strop et al., “Location matters: site of conjugation modulates stability and pharmacokinetics of antibody drug conjugates,” Chemistry and Biology 20(2) 161-167 (2013))

In some embodiments, the polynucleotide is conjugated to the targeting molecule by a method as described in PCT Publication No. WO2014/140317 (incorporated herein by reference in its entirety), which utilizes a sequence-specific transpeptidase. In some embodiments, the polynucleotide is conjugated to the targeting molecule by a method as described in U.S. Patent Publication Nos. 2015/0105539 and 2015/0105540, each of which is incorporated herein by reference in its entirety.

In some embodiments, each targeting molecule is conjugated to between one and eight polynucleotide molecules (i.e. a Drug:Antibody Ratio (DAR) between 1 and 8). In some embodiments, each targeting molecule is conjugated to one polynucleotide molecule (DAR of 1). In some embodiments, each targeting molecule is conjugated to two polynucleotide molecules (DAR of 2). In some embodiments, each targeting molecule is conjugated to three polynucleotide molecules (DAR of 3). In some embodiments, each targeting molecule is conjugated to four polynucleotide molecules (DAR of 4). In some embodiments, each targeting molecule is conjugated to five polynucleotide molecules (DAR of 5). In some embodiments, each targeting molecule is conjugated to six polynucleotide molecules (DAR of 6). In some embodiments, each targeting molecule is conjugated to seven polynucleotide molecules (DAR of 7). In some embodiments, each targeting molecule is conjugated to eight polynucleotide molecules (DAR of 8).

In some embodiments, the polynucleotide-conjugated targeting molecule has a molecular weight greater than about 30 kDa. In some embodiments, the polynucleotide-conjugated targeting molecule has a molecular weight greater than about 40 kDa. The polynucleotide-conjugated targeting molecule may have a molecular weight greater than about 50 kDa. In some embodiments, the polynucleotide-conjugated targeting molecule has a molecular weight greater than about 60 kDa. In some embodiments, the polynucleotide-conjugated targeting molecule has a molecular weight no greater than about 7,500 kDa.

In some embodiments, the polynucleotide-conjugated targeting molecule has a molecular weight greater than 30 kDa. In some embodiments, the polynucleotide-conjugated targeting molecule has a molecular weight greater than 40 kDa. The polynucleotide-conjugated targeting molecule may have a molecular weight greater than 50 kDa. In some embodiments, the polynucleotide-conjugated targeting molecule has a molecular weight greater than 60 kDa. In some embodiments, the polynucleotide-conjugated targeting molecule has a molecular weight no greater than 7,500 kDa.

### Polynucleotide Conjugates

Another aspect of this disclosure provides polynucleotide conjugates. In some embodiments, the polynucleotide conjugate comprise a polynucleotide conjugated to a targeting molecule. In such aspects, the targeting molecule may also be referred to as targeting agent component, whereas the polynucleotide may be referred to as an example of an active agent component. I.e., disclosed herein are (therapeutic) targeting agents comprising an active agent component and a targeting agent component.

In some embodiments, the targeting moiety (in aspects also referred to as targeting agent component) comprises amino acids, peptides, polypeptides, proteins, antibodies, antigens, toxins, hormones, lipids, nucleotides, nucleosides, sugars, carbohydrates, polymers such as polyethylene glycol and polypropylene glycol, as well as analogs or derivatives of all of these classes of substances. Additional examples of targeting moiety also include steroids, such as cholesterol, phospholipids, di- and triacylglycerols, fatty acids, hydrocarbons (e.g., saturated, unsaturated, or contains substitutions), enzyme substrates, biotin, digoxigenin, and polysaccharides. In some embodiments, the targeting moiety is an antibody or binding fragment thereof.

The targeting molecule (in aspects also referred to as targeting agent component) may be an antibody or an antigen-binding fragment thereof, or a binding protein. In some embodiments, the targeting molecule is an antibody or an antigen binding fragment thereof (e.g. a polynucleotide-antibody conjugate). In some embodiments, the antibody or binding fragment thereof is a human antibody or an antigen-binding fragment thereof, a humanized antibody or an antigen-binding fragment thereof, a murine antibody or an antigen-binding fragment thereof, a chimeric antibody or an antigen-binding fragment thereof, a monoclonal antibody or an antigen-binding fragment thereof, a monovalent Fab′, a divalent Fab2, a F(ab)′3 fragment, a single-chain variable fragment (scFv), a bis-scFv, a (scFv)2, a diabody, a minibody, a immunoglobulin single variable domain (ISV) such as an NANOBODY® molecule, a triabody, a tetrabody, a disulfide stabilized Fv protein (dsFv), a single-domain antibody (sdAb), an Ig NAR, a vNAR, a mutein based on Tenascin C (also known as a Centyrin molecule), a camelid antibody or an antigen-binding fragment thereof, a bispecific antibody or an antigen binding fragment thereof, or a chemically modified derivative thereof. In some embodiments, the antibody or antigen-binding fragment thereof is selected from the group consisting of a monoclonal antibody, a bispecific antibody, a Fab, a Fab-Fc, a Fv, a single chain Fv (scFv), a diabody, a minibody, and a immunoglobulin single variable domain (ISV) such as an NANOBODY® molecule. In some embodiments, the antibody or antigen-binding fragment thereof is a monoclonal antibody. In some embodiments, the antibody or antigen-binding fragment thereof is a bispecific antibody. Non-limiting examples of bispecific antibodies in bispecific T-cell engagers (BiTEs) and a dual-affinity retargeting antibodies (DARTs). In some embodiments, the bispecific antibody is a trifunctional antibody or a bispecific mini-antibody. In some embodiments, the bispecific antibody is a trifunctional antibody. In some embodiments, the trifunctional antibody is a full-length monoclonal antibody comprising binding sites for two different antigens. In some embodiments, the bispecific antibody is a bispecific mini-antibody. In some embodiments, the bispecific mini-antibody comprises divalent Fab2, F(ab)′3 fragments, bis-scFv, (scFv)2, diabody, minibody, triabody, tetrabody or a bi-specific T-cell engager (BiTE). In some embodiments, the bi-specific T-cell engager is a fusion protein that contains two single-chain variable fragments (scFvs) in which the two scFvs target epitopes of two different antigens.

In some embodiments, the antibody or antigen-binding fragment thereof is a Fab. In some embodiments, the antibody or antigen-binding fragment thereof is a Fab-Fc. In some embodiments, the antibody or antigen-binding fragment thereof is a Fv. In some embodiments, the antibody or antigen-binding fragment thereof is a single chain Fv (scFv). In some embodiments, when the antibody or antigen-binding portion is a scFv, the polynucleotide does not comprise a cross-linking residue. In some embodiments, when the antibody or antigen-binding portion is a scFv, the polynucleotide does not comprise a cysteine. In some embodiments, the antibody or antigen-binding fragment thereof is a diabody. In some embodiments, the antibody or antigen-binding fragment thereof is a minibody. In some embodiments, the antibody or antigen-binding fragment thereof is a immunoglobulin single variable domain (ISV) such as an NANOBODY® molecule. The NANOBODY® molecule may be a NANOBODY® molecule-HSA.

In some embodiments, the antibody or antigen-binding fragment thereof is an IgG molecule or is derived from an IgG molecule. The IgG molecule may be an IgG1 or an IgG4 molecule. The antibody or antigen-binding fragment thereof may be an IgG1 molecule or derived therefrom. The antibody or antigen-binding fragment thereof may be an IgG2 molecule or derived therefrom. The antibody or antigen-binding fragment thereof may be an IgG3 molecule or derived therefrom. The antibody or antigen-binding fragment thereof may be an IgG4 molecule or derived therefrom.

In some embodiments, the targeting molecule is a binding protein. The binding protein may be a soluble receptor or a soluble ligand. In some embodiments, the soluble receptor comprises the extracellular domain of a receptor. In some embodiments, the soluble receptor is a Fc fusion protein.

In some embodiments, the targeting molecule is a plasma protein. In some embodiments, the plasma protein comprises albumin. In some embodiments, the albumin is conjugated by one or more of the conjugation chemistries disclosed herein to a polynucleotide. In some instances, the albumin is conjugated by native ligation chemistry to a polynucleotide. In some instances, albumin is conjugated by lysine conjugation to a polynucleotide.

In some instances, the targeting molecule is a steroid. Non-limiting exemplary steroids include cholesterol, phospholipids, di- and triacylglycerols, fatty acids, hydrocarbons that are saturated, unsaturated, comprise substitutions, or combinations thereof. In some embodiments, the steroid is cholesterol or a cholesterol derivative. In some embodiments, the targeting molecule is cholesterol. In some embodiments, the steroid is conjugated by one or more of the conjugation chemistries disclosed herein to a polynucleotide. In some embodiments, the steroid is conjugated by native ligation chemistry to a polynucleotide.

In some embodiments, the targeting molecule is a polymer, including but not limited to polynucleotide aptamers that bind to specific surface markers on cells. In some embodiments, the targeting molecule is a polynucleotide that does not hybridize to a target gene or mRNA, but instead is capable of selectively binding to a cell surface marker similarly to an antibody binding to its specific epitope of a cell surface marker.

In some embodiments, the targeting molecule is a polypeptide. In some embodiments, the polypeptide has a size between about 1 and about 3 kDa. In some embodiments, the polypeptide has a size between about 1.2 and about 2.8 kDa, about 1.5 and about 2.5 kDa, or about 1.5 and about 2 kDa. In some embodiments, the polypeptide is a bicyclic polypeptide. In some embodiments, the bicyclic polypeptide is a constrained bicyclic polypeptide. In some embodiments, the targeting molecule is a bicyclic polypeptide (e.g., bicycles from Bicycle Therapeutics).

In additional embodiments, the targeting molecule is a small molecule. In some embodiments, the small molecule is an antibody-recruiting small molecule. In some embodiments, the antibody-recruiting small molecule comprises a target-binding terminus and an antibody-binding terminus, in which the target-binding terminus is capable of recognizing and interacting with a cell surface receptor.

In some embodiments, the targeting molecule is a therapeutically active molecule or a biologically active molecule.

In some embodiments, the active agent component is a polynucleotide.

In some embodiments, the polynucleotide comprises RNA, DNA or a combination thereof. In some cases, the polynucleotide comprises RNA. In some cases, the polynucleotide comprises DNA. In some cases, the polynucleotide comprises RNA and DNA. In some embodiments, the polynucleotide comprises combinations of DNA, RNA and/or artificial nucleotide analogues. In some embodiments, the polynucleotide is a regulatory non-coding RNA (ncRNA). In some embodiments, the ncRNA comprises short non-coding RNA sequences expressed in a genome that regulates expression or function of other biomolecules in mammalian cells. An ncRNA is generally <200 nucleotides in length and can be single stranded or double stranded and may form non-linear secondary or tertiary structures. An ncRNA can comprise exogenously derived small interfering RNA (siRNA), MicroRNA (miRNA), small nuclear RNA (U-RNA), Small nucleolar RNA (snoRNA), Piwi-interacting RNA (piRNA), repeat associated small interfering RNA (rasiRNA), small rDNA-derived RNA (srRNA), transfer RNA derived small RNA (tsRNA), ribosomal RNA derived small RNA (rsRNA), large non-coding RNA derived small RNA (lncsRNA), or a messenger RNA derived small RNA (msRNA). In some embodiments, the polynucleotide is an engineered polynucleotide. The engineered polynucleotide may comprise DNA or RNA. In some embodiments, the engineered polynucleotide comprises a plurality of nucleotides. In some embodiments, the engineered polynucleotide comprises an artificial nucleotide analogue. In some embodiments, the engineered polynucleotide comprises DNA. In some embodiments, the DNA is genomic DNA, cell-free DNA, cDNA, fetal DNA, viral DNA, or maternal DNA. In some embodiments, the engineered polynucleotide comprises RNA. In some embodiments, the RNA is an siRNA, an ncRNA mimic, a short-harpin RNA (shRNA), a dicer-dependent siRNA (di-siRNA), an antisense oligonucleotide (ASO), a gapmer, a mixmer, double-stranded RNAs (dsRNA), single stranded RNAi, (ssRNAi), DNA-directed RNA interference (ddRNAi), an RNA activating oligonucleotide (RNAa), an aptamer, or an exon skipping oligonucleotide. In some embodiments, the engineered polynucleotide comprises a completely synthetic miRNA. A completely synthetic miRNA is one that is not derived or based upon an ncRNA. Instead, a completely synthetic miRNA may be based upon an analysis of multiple potential target sequences or may be based upon isolated natural non-coding sequences that are not ncRNAs. In some embodiments, the polynucleotide is selected from the group consisting of a siRNA, a miRNA, a miRNA mimic, an antisense oligonucleotide (ASO), an mRNA, and a guide RNA. The polynucleotide may be a siRNA. In some embodiments, the polynucleotide is a miRNA. In some embodiments, the polynucleotide is a miRNA mimic.

In some embodiments, the active agent component is an ASO. In some embodiment the ASO can target and repress multiple genes related to a disorder. In some embodiments, the ASO targets an autosomal dominant mutant gene that causes a genetic disorder. In some embodiments, the ASO targets DMPK. In some embodiments, the ASO targets CAPN3. The ASO may target DUX4. DUX4-targeted ASOs are known in the art. See, e.g., WO 2021/203043 and U.S. Provisional Patent Application No. 63/221,568, each of which is incorporated herein by reference in its entirety. Additional non-limiting examples of DUX4-targeted ASOs are provided in Table 1, supra. In some embodiments, the DUX4-targeted ASO is selected from the group consisting of ASDX2, ASDX4, ASDX23, ASDX26, and ASDX32. In some embodiments, the DUX4-targeted ASO is ASDX2. In some embodiments, the DUX4-targeted ASO is ASDX4. In some embodiments, the DUX4-targeted ASO is ASDX23. In some embodiments, the DUX4-targeted ASO is ASDX26. In some embodiments, the DUX4-targeted ASO is ASDX32.

In some embodiments, the polynucleotide comprises a siRNA, a miRNA, a miRNA mimic, an ASO, or a guide RNA that targets DUX4, DMPK or CAPN3. In some embodiments, the polynucleotide comprises a siRNA that targets DUX4. In some embodiments, the polynucleotide comprises a miRNA that targets DUX4. In some embodiments, the polynucleotide comprises a miRNA mimic that targets DUX4. In some embodiments, the polynucleotide comprises an ASO that targets DUX4. In some embodiments, the polynucleotide comprises a guide RNA that targets DUX4. In some embodiments, the polynucleotide comprises a siRNA that targets DMPK. In some embodiments, the polynucleotide comprises a miRNA that targets DMPK. In some embodiments, the polynucleotide comprises a miRNA mimic that targets DMPK. In some embodiments, the polynucleotide comprises an ASO that targets DMPK. In some embodiments, the polynucleotide comprises a siRNA that targets CAPN3. In some embodiments, the polynucleotide comprises a miRNA that targets CAPN3. In some embodiments, the polynucleotide comprises a miRNA mimic that targets CAPN3. In some embodiments, the polynucleotide comprises an ASO that targets CAPN3.

In some embodiments, the polynucleotide is a coding RNA. In some embodiments, the polynucleotide is a mRNA. In some embodiments, the polynucleotide is a non-coding RNA. In some embodiments, the polynucleotide is a long non-coding RNA. In some embodiments, the polynucleotide is a guide RNA.

In some embodiments, the polynucleotide comprises one or more artificial nucleotide analogues. In some embodiments, one or more of the artificial nucleotide analogues described herein are resistant toward nucleases such as for example ribonuclease such as RNase, deoxyribonuclease such as DNase, or exonuclease such as 5′-3′ exonuclease and 3′-5′ exonuclease when compared to natural polynucleotides. In some embodiments, artificial nucleotide analogues comprising 2′-O-methyl, 2′-O-methoxyethyl (2′-O-MOE), 2′-O-aminopropyl, 2′-deoxy, T-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), T-O-dimethylaminoethyloxyethyl (2′-O-DMAEOE), or 2′-O—N-methylacetamido (2′-O-NMA) modified, LNA, ENA, PNA, HNA, morpholino, methylphosphonate nucleotides, thiolphosphonate nucleotides, 2′-fluoro N3-P5′-phosphoramidites, or combinations thereof are resistant toward nucleases such as for example ribonuclease such as RNase, deoxyribonuclease such as DNase, or exonuclease such as 5′-3′ exonuclease and 3′-5′ exonuclease. In some embodiments, 2′-O-methyl modified polynucleotide is nuclease resistant (e.g., RNase, DNase, 5′-3′ exonuclease or 3′-5′ exonuclease resistant). In some embodiments, 2′O-methoxyethyl (2′-O-MOE) modified polynucleotide is nuclease resistant (e.g., RNase, DNase, 5′-3′ exonuclease or 3′-5′ exonuclease resistant). In some embodiments, 2′-O-aminopropyl modified polynucleotide is nuclease resistant (e.g., RNase, DNase, 5′-3′ exonuclease or 3′-5′ exonuclease resistant). In some embodiments, 2′-deoxy modified polynucleotide is nuclease resistant (e.g., RNase, DNase, 5′-3′ exonuclease or 3′-5′ exonuclease resistant). In some embodiments, T-deoxy-2′-fluoro modified polynucleotide is nuclease resistant (e.g., RNase, DNase, 5′-3′ exonuclease or 3′-5′ exonuclease resistant). In some embodiments, 2′-O-aminopropyl (2′-O-AP) modified polynucleotide is nuclease resistant (e.g., RNase, DNase, 5′-3′ exonuclease or 3′-5′ exonuclease resistant). In some embodiments, 2′-O-dimethylaminoethyl (2′-O-DMAOE) modified polynucleotide is nuclease resistant (e.g., RNase, DNase, 5′-3′ exonuclease or 3′-5′ exonuclease resistant). In some embodiments, 2′-O-dimethylaminopropyl (2′-O-DMAP) modified polynucleotide is nuclease resistant (e.g., RNase, DNase, 5′-3′ exonuclease or 3′-5′ exonuclease resistant). In some embodiments, T-O-dimethylaminoethyloxyethyl (2′-O-DMAEOE) modified polynucleotide is nuclease resistant (e.g., RNase, DNase, 5′-3′ exonuclease or 3′-5′ exonuclease resistant). In some embodiments, 2′-O—N-methylacetamido (2′-O-NMA) modified polynucleotide is nuclease resistant (e.g., RNase, DNase, 5′-3′ exonuclease or 3′-5′ exonuclease resistant). In some embodiments, LNA-modified polynucleotide is nuclease resistant (e.g., RNase, DNase, 5′-3′ exonuclease or 3′-5′ exonuclease resistant). In some embodiments, ENA-modified polynucleotide is nuclease resistant (e.g., RNase, DNase, 5′-3′ exonuclease or 3′-5′ exonuclease resistant). In some embodiments, HNA-modified polynucleotide is nuclease resistant (e.g., RNase, DNase, 5′-3′ exonuclease or 3′-5′ exonuclease resistant). Morpholinos may be nuclease resistant (e.g., RNase, DNase, 5′-3′ exonuclease or 3′-5′ exonuclease resistant). In some embodiments, PNA-modified polynucleotide is resistant to nucleases (e.g., RNase, DNase, 5′-3′ exonuclease or 3′-5′ exonuclease resistant). In some embodiments, methylphosphonate nucleotide-modified polynucleotide is nuclease resistant (e.g., RNase, DNase, 5′-3′ exonuclease or 3′-5′ exonuclease resistant). In some embodiments, thiolphosphonate nucleotide-modified polynucleotide is nuclease resistant (e.g., RNase, DNase, 5′-3′ exonuclease or 3′-5′ exonuclease resistant). In some embodiments, polynucleotide comprising 2′-fluoro N3-P5′-phosphoramidites is nuclease resistant (e.g., RNase, DNase, 5′-3′ exonuclease or 3′-5′ exonuclease resistant). In some embodiments, the 5′ conjugates described herein inhibit 5′-3′ exonucleolytic cleavage. In some embodiments, the 3′ conjugates described herein inhibit 3′-5′ exonucleolytic cleavage.

In some embodiments, one or more of the artificial nucleotide analogues described herein have increased binding affinity toward their mRNA target relative to an equivalent natural polynucleotide. In some embodiments, the artificial nucleotide analogue comprises a nucleic acid with a modification at a 2′ hydroxyl group of the ribose moiety. In some embodiments, the modification includes an H, OR, R, halo, SH, SR, NH2, NHR, NR2, or CN, wherein R is an alkyl moiety. Exemplary alkyl moieties include, but are not limited to, halogens, sulfurs, thiols, thioethers, thioesters, amines (primary, secondary, or tertiary), amides, ethers, esters, alcohols and oxygen. In some embodiments, the alkyl moiety further comprises a modification. In some embodiments, the modification comprises an azo group, a keto group, an aldehyde group, a carboxyl group, a nitro group, a nitroso, group, a nitrile group, a heterocycle (e.g., imidazole, hydrazino or hydroxylamino) group, an isocyanate or cyanate group, or a sulfur containing group (e.g., sulfoxide, sulfone, sulfide, or disulfide). In some embodiments, the alkyl moiety further comprises a hetero substitution. In some embodiments, the carbon of the heterocyclic group is substituted by a nitrogen, oxygen or sulfur. In some embodiments, the heterocyclic substitution includes but is not limited to, morpholino, imidazole, and pyrrolidino. The one or more of the artificial nucleotide analogues comprising 2′-O-methyl, 2′-O-methoxyethyl (2′-O-MOE), 2′-O-aminopropyl, 2′-deoxy, T-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), T-O-dimethylaminoethyloxyethyl (2′-O-DMAEOE), or 2′-O—N-methylacetamido (2′-O-NMA) modified, LNA, ENA, PNA, HNA, morpholino, methylphosphonate nucleotides, thiolphosphonate nucleotides, or 2′-fluoro N3-P5′-phosphoramidites can have increased binding affinity toward their mRNA target relative to an equivalent natural polynucleotide. In some embodiments, 2′-O-methyl modified polynucleotide has increased binding affinity toward their mRNA target relative to an equivalent natural polynucleotide. In some embodiments, 2′-O-methoxyethyl (2′-O-MOE) modified polynucleotide has increased binding affinity toward their mRNA target relative to an equivalent natural polynucleotide. In some embodiments, 2′-O-aminopropyl modified polynucleotide has increased binding affinity toward their mRNA target relative to an equivalent natural polynucleotide. In some embodiments, 2′-deoxy modified polynucleotide has increased binding affinity toward their mRNA target relative to an equivalent natural polynucleotide. In some embodiments, T-deoxy-2′-fluoro modified polynucleotide has increased binding affinity toward their mRNA target relative to an equivalent natural polynucleotide. In some embodiments, 2′-O-aminopropyl (2′-O-AP) modified polynucleotide has increased binding affinity toward their mRNA target relative to an equivalent natural polynucleotide. In some embodiments, 2′-O-dimethylaminoethyl (2′-O-DMAOE) modified polynucleotide has increased binding affinity toward their mRNA target relative to an equivalent natural polynucleotide. In some embodiments, 2′-O-dimethylaminopropyl (2′-O-DMAP) modified polynucleotide has increased binding affinity toward their mRNA target relative to an equivalent natural polynucleotide. In some embodiments, T-O-dimethylaminoethyloxyethyl (2′-O-DMAEOE) modified polynucleotide has increased binding affinity toward their mRNA target relative to an equivalent natural polynucleotide. In some embodiments, 2′-O—N-methylacetamido (2′-O-NMA) modified polynucleotide has increased binding affinity toward their mRNA target relative to an equivalent natural polynucleotide. In some embodiments, LNA-modified polynucleotide has increased binding affinity toward their mRNA target relative to an equivalent natural polynucleotide. In some embodiments, ENA-modified polynucleotide has increased binding affinity toward their mRNA target relative to an equivalent natural polynucleotide. In some embodiments, PNA-modified polynucleotide has increased binding affinity toward their mRNA target relative to an equivalent natural polynucleotide. In some embodiments, HNA-modified polynucleotide has increased binding affinity toward their mRNA target relative to an equivalent natural polynucleotide. In some embodiments, morpholino-modified polynucleotide has increased binding affinity toward their mRNA target relative to an equivalent natural polynucleotide. In some embodiments, methylphosphonate nucleotide-modified polynucleotide has increased binding affinity toward their mRNA target relative to an equivalent natural polynucleotide. In some embodiments, thiolphosphonate nucleotide-modified polynucleotide has increased binding affinity toward their mRNA target relative to an equivalent natural polynucleotide. In some embodiments, polynucleotide comprising 2′-fluoro N3-P5′-phosphoramidites has increased binding affinity toward their mRNA target relative to an equivalent natural polynucleotide. In some embodiments, the increased affinity is illustrated with a lower Kd, a higher melt temperature (Tm), or a combination thereof.

In some embodiments, the artificial nucleotide analogues include 2′-O-methyl, 2′-O-methoxyethyl (2′-O-MOE), 2′-O-aminopropyl, 2′-deoxy, T-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), T-O-dimethylaminoethyloxyethyl (2′-O-DMAEOE), or 2′-O—N-methylacetamido (2′-O-NMA) modified, LNA, ENA, PNA, HNA, morpholino, methylphosphonate nucleotides, thiolphosphonate nucleotides, 2′-fluoro N3-P5′-phosphoramidites, or a combination thereof.

In some embodiments, the artificial nucleotide analogue comprises a modified base such as, but not limited to, 5-propynyluridine, 5-propynylcytidine, 6-methyladenine, 6-methylguanine, N,N,-dimethyladenine, 2-propyladenine, 2propylguanine, 2-aminoadenine, 1-methylinosine, 3-methyluridine, 5-methylcytidine, 5-methyluridine and other nucleotides having a modification at the 5 position, 5-(2-amino) propyl uridine, 5-halocytidine, 5-halouridine, 4-acetylcytidine, 1-methyladenosine, 2-methyladenosine, 3-methylcytidine, 6-methyluridine, 2-methylguanosine, 7-methylguanosine, 2, 2-dimethylguanosine, 5-methylaminoethyluridine, 5-methyloxyuridine, deazanucleotides (such as 7-deaza-adenosine, 6-azouridine, 6-azocytidine, or 6-azothymidine), 5-methyl-2-thiouridine, other thio bases (such as 2-thiouridine, 4-thiouridine, and 2-thiocytidine), dihydrouridine, pseudouridine, queuosine, archaeosine, naphthyl and substituted naphthyl groups, any O- and N-alkylated purines and pyrimidines (such as N6-methyladenosine, 5-methylcarbonylmethyluridine, uridine 5-oxyacetic acid, pyridine-4-one, or pyridine-2-one), phenyl and modified phenyl groups such as aminophenol or 2,4,6-trimethoxy benzene, modified cytosines that act as G-clamp nucleotides, 8-substituted adenines and guanines, 5-substituted uracils and thymines, azapyrimidines, carboxyhydroxyalkyl nucleotides, carboxyalkylaminoalkyl nucleotides, and alkylcarbonylalkylated nucleotides. Modified nucleotides also include those nucleotides that are modified with respect to the sugar moiety, as well as nucleotides having sugars or analogs thereof that are not ribosyl. For example, the sugar moieties, In some embodiments are or are based on, mannoses, arabinoses, glucopyranoses, galactopyranoses, 4′-thioribose, and other sugars, heterocycles, or carbocycles. The term nucleotide also includes what are known in the art as universal bases. By way of example, universal bases include but are not limited to 3-nitropyrrole, 5-nitroindole, or nebularine.

In some embodiments, the polynucleotide comprises one or more phosphorothioate internucleotide linkages. In some embodiments, the polynucleotide comprises 2′-5′ internucleotide linkages. In some embodiments, the 2′-5′ internucleotide linkage(s) is at the 3′-end, the 5′-end, or both of the 3′- and 5′-ends of one or both sequence strands. In some embodiments, the 2′-5′ internucleotide linkage(s) is present at various other positions within one or both sequence strands. In some embodiments, the polynucleotide comprises a terminal cap molecule at the 3′-end, the 5′-end, or both of the 3′- and 5′-ends.

In some embodiments, the targeting molecule and the polynucleotide combined to provide a synergistic therapeutic or biological effect.

In some embodiments, the polynucleotide is conjugated directly to the targeting molecule (such as the targeting agent component described herein). The polynucleotide may be conjugated to the targeting molecule via a linker. Suitable linkers for conjugating polynucleotides to targeting molecules are known in the art. See, e.g., WO 2017/173408, incorporated herein by reference in its entirety. In some embodiments, the linker is a hydrophobic linker. The linker may be a peptide linker. In some embodiments, the linker is a chemical linker. The chemical linker may be a polymeric linker. In some embodiments, the chemical linker is linear. In some embodiments, the chemical linker is cyclic.

In some embodiments, the polymeric linker comprises PEG, a sugar, a fatty acid, a phosphate, a pyrophosphate or a polysarcosine. In some embodiments, the polymeric linker comprises PEG. In some embodiments, the polymeric linker comprises a sugar. In some embodiments, the polymeric linker comprises a fatty acid. In some embodiments, the polymeric linker comprises a phosphate. In some embodiments, the polymeric linker comprises a pyrophosphate. In some embodiments, the polymeric linker comprises a polysarcosine. The linker may be a high molecular weight PEG linker. In some embodiments, the high molecular weight PEG linker comprises between 1,000 and 5,000 PEG monomers (i.e. is between PEG1k and PEG5k). In some embodiments, the high molecular weight PEG linker is PEG1k. In some embodiments, the high molecular weight PEG linker is PEG1.5k. In some embodiments, the high molecular weight PEG linker is PEG2k. In some embodiments, the high molecular weight PEG linker is PEG3k. In some embodiments, the high molecular weight PEG linker is PEG4k. In some embodiments, the high molecular weight PEG linker is PEG5k.

In some embodiments, the linker is a low molecular weight PEG linker. In some embodiments, the low molecular weight PEG linker comprises between 4 and 100 PEG monomers (i.e. is between PEG4 and PEG100). In some embodiments, the low molecular PEG linker is between PEG12 and PEG48. In some embodiments, the low molecular PEG linker is between PEG12 and PEG24. In some embodiments, the low molecular PEG linker is between PEG12 and PEG18. In some embodiments, the low molecular PEG linker is between PEG6 and PEG18. In some embodiments, the low molecular weight PEG linker is PEG4. In some embodiments, the low molecular weight PEG linker is PEG5. In some embodiments, the low molecular weight PEG linker is PEG6. In some embodiments, the low molecular weight PEG linker is PEG7. In some embodiments, the low molecular weight PEG linker is PEG8. In some embodiments, the low molecular weight PEG linker is PEG9. In some embodiments, the low molecular weight PEG linker is PEG10. In some embodiments, the low molecular weight PEG linker is PEG11. In some embodiments, the low molecular weight PEG linker is PEG12. In some embodiments, the low molecular weight PEG linker is PEG13. In some embodiments, the low molecular weight PEG linker is PEG14. In some embodiments, the low molecular weight PEG linker is PEG15. In some embodiments, the low molecular weight PEG linker is PEG16. In some embodiments, the low molecular weight PEG linker is PEG17. In some embodiments, the low molecular weight PEG linker is PEG18. In some embodiments, the low molecular weight PEG linker is PEG19. In some embodiments, the low molecular weight PEG linker is PEG20. In some embodiments, the low molecular weight PEG linker is PEG21. In some embodiments, the low molecular weight PEG linker is PEG22. In some embodiments, the low molecular weight PEG linker is PEG23. In some embodiments, the low molecular weight PEG linker is PEG24. In some embodiments, the low molecular weight PEG linker is PEG25. In some embodiments, the low molecular weight PEG linker is PEG26. In some embodiments, the low molecular weight PEG linker is PEG27. In some embodiments, the low molecular weight PEG linker is PEG28. In some embodiments, the low molecular weight PEG linker is PEG29. In some embodiments, the low molecular weight PEG linker is PEG30. In some embodiments, the low molecular weight PEG linker is PEG31. In some embodiments, the low molecular weight PEG linker is PEG32. In some embodiments, the low molecular weight PEG linker is PEG33. In some embodiments, the low molecular weight PEG linker is PEG34. In some embodiments, the low molecular weight PEG linker is PEG35. In some embodiments, the low molecular weight PEG linker is PEG36. In some embodiments, the low molecular weight PEG linker is PEG37. In some embodiments, the low molecular weight PEG linker is PEG38. In some embodiments, the low molecular weight PEG linker is PEG39. In some embodiments, the low molecular weight PEG linker is PEG40. In some embodiments, the low molecular weight PEG linker is PEG41. In some embodiments, the low molecular weight PEG linker is PEG42. In some embodiments, the low molecular weight PEG linker is PEG43. In some embodiments, the low molecular weight PEG linker is PEG44. In some embodiments, the low molecular weight PEG linker is PEG45. In some embodiments, the low molecular weight PEG linker is PEG46. In some embodiments, the low molecular weight PEG linker is PEG47. In some embodiments, the low molecular weight PEG linker is PEG48. In some embodiments, the low molecular weight PEG linker is PEG49. In some embodiments, the low molecular weight PEG linker is PEG50. In some embodiments, the low molecular weight PEG linker is PEG51. In some embodiments, the low molecular weight PEG linker is PEG52. In some embodiments, the low molecular weight PEG linker is PEG53. In some embodiments, the low molecular weight PEG linker is PEG54. In some embodiments, the low molecular weight PEG linker is PEG55. In some embodiments, the low molecular weight PEG linker is PEG56. In some embodiments, the low molecular weight PEG linker is PEG57. In some embodiments, the low molecular weight PEG linker is PEG58. In some embodiments, the low molecular weight PEG linker is PEG59. In some embodiments, the low molecular weight PEG linker is PEG60. In some embodiments, the low molecular weight PEG linker is PEG61. In some embodiments, the low molecular weight PEG linker is PEG62. In some embodiments, the low molecular weight PEG linker is PEG63. In some embodiments, the low molecular weight PEG linker is PEG64. In some embodiments, the low molecular weight PEG linker is PEG65. In some embodiments, the low molecular weight PEG linker is PEG66. In some embodiments, the low molecular weight PEG linker is PEG67. In some embodiments, the low molecular weight PEG linker is PEG68. In some embodiments, the low molecular weight PEG linker is PEG69. In some embodiments, the low molecular weight PEG linker is PEG70. In some embodiments, the low molecular weight PEG linker is PEG71. In some embodiments, the low molecular weight PEG linker is PEG72. In some embodiments, the low molecular weight PEG linker is PEG73. In some embodiments, the low molecular weight PEG linker is PEG74. In some embodiments, the low molecular weight PEG linker is PEG75. In some embodiments, the low molecular weight PEG linker is PEG76. In some embodiments, the low molecular weight PEG linker is PEG77. In some embodiments, the low molecular weight PEG linker is PEG78. In some embodiments, the low molecular weight PEG linker is PEG79. In some embodiments, the low molecular weight PEG linker is PEG80. In some embodiments, the low molecular weight PEG linker is PEG81. In some embodiments, the low molecular weight PEG linker is PEG82. In some embodiments, the low molecular weight PEG linker is PEG83. In some embodiments, the low molecular weight PEG linker is PEG84. In some embodiments, the low molecular weight PEG linker is PEG85. In some embodiments, the low molecular weight PEG linker is PEG86. In some embodiments, the low molecular weight PEG linker is PEG87. In some embodiments, the low molecular weight PEG linker is PEG88. In some embodiments, the low molecular weight PEG linker is PEG89. In some embodiments, the low molecular weight PEG linker is PEG90. In some embodiments, the low molecular weight PEG linker is PEG91. In some embodiments, the low molecular weight PEG linker is PEG92. In some embodiments, the low molecular weight PEG linker is PEG93. In some embodiments, the low molecular weight PEG linker is PEG94. In some embodiments, the low molecular weight PEG linker is PEG95. In some embodiments, the low molecular weight PEG linker is PEG96. In some embodiments, the low molecular weight PEG linker is PEG97. In some embodiments, the low molecular weight PEG linker is PEG98. In some embodiments, the low molecular weight PEG linker is PEG99. In some embodiments, the low molecular weight PEG linker is PEG100.

In some embodiments, the linker is non-cleavable. In some embodiments, the linker is cleavable. The linker may be cleavable in vivo. In some embodiments, the cleavable linker is selected from the group consisting of a disulfide linker, a self-immolative peptide polymer hybrid, and a sulfatase-promoted arylsulfate linker. In some embodiments, the cleavable linker is a disulfide linker. The cleavable linker may be a self-immolative peptide polymer hybrid. In some embodiments, the cleavable linker is a sulfatase-promoted arylsulfate linker. In some embodiments, the self-immolative peptide polymer hybrid comprises glucuronic acid, para-amino-benzoyloxy (PAB), 7-amino-3-hydroxyethyl-coumarin (7-AHC), or Fe(II)-reactive 1,2,4-trioxolane scaffold (TRX). In some embodiments, the self-immolative peptide polymer hybrid comprises glucuronic acid. In some embodiments, the self-immolative peptide polymer hybrid comprises para-amino-benzoyloxy (PAB). In some embodiments, the self-immolative peptide polymer hybrid comprises 7-amino-3-hydroxyethyl-coumarin (7-AHC). In some embodiments, the self-immolative peptide polymer hybrid comprises Fe(II)-reactive 1,2,4-trioxolane scaffold (TRX).

In some embodiments, the cleavable linker is cleaved through reduction, hydrolysis, proteolysis, photo cleavage, chemical cleavage, enzymatic cleavage, or bio-orthogonal-cleavage. In some embodiments, the cleavable linker is cleaved through reduction. In some embodiments, the cleavable linker is cleaved through hydrolysis. In some embodiments, the cleavable linker is cleaved through proteolysis. In some embodiments, the cleavable linker is cleaved through photo cleavage. In some embodiments, the cleavable linker is cleaved through chemical cleavage. The chemical cleavage may be by Fe II mediated β elimination of TRX. In some embodiments, the cleavable linker is cleaved through enzymatic cleavage. The enzymatic cleavage may be by non-proteolytic sulfatase, β-galactosidase/glucuronidase or pyrophosphatase. In some embodiments, the enzymatic cleavage is by non-proteolytic sulfatase. In some embodiments, the enzymatic cleavage is by β-galactosidase/glucuronidase. In some embodiments, the enzymatic cleavage is by pyrophosphatase. In some embodiments, the cleavable linker is cleaved through bio-orthogonal-cleavage. The bio-orthogonal cleavage may be by Cu I-BTTAA or free copper ion mediated cleavage. In some embodiments, the linker is an acid cleavable linker.

In some embodiments, the linker includes a C1-C6 alkyl group (e.g., a C5, C4, C3, C2, or C1 alkyl group). In some embodiments, the linker includes homobifunctional cross linkers, heterobifunctional cross linkers, and the like. In some embodiments, the liker is a traceless linker (or a zero-length linker). In some embodiments, the linker is a non-polymeric linker. In some embodiments, the linker is a non-peptide linker or a linker that does not contain an amino acid residue.

In some embodiments, the linker comprises a homobifunctional linker. Exemplary homobifunctional linkers include, but are not limited to, Lomant's reagent dithiobis (succinimidylpropionate) DSP, 3,3′-dithiobis(sulfosuccinimidyl proprionate (DTSSP), disuccinimidyl suberate (DSS), bis(sulfosuccinimidyl)suberate (BS), disuccinimidyl tartrate (DST), disulfosuccinimidyl tartrate (sulfo DST), ethylene glycobis(succinimidylsuccinate) (EGS), disuccinimidyl glutarate (DSG), N,N′-disuccinimidyl carbonate (DSC), dimethyl adipimidate (DMA), dimethyl pimelimidate (DMP), dimethyl suberimidate (DMS), dimethyl-3,3′-dithiobispropionimidate (DTBP), 1,4-di-3′-(2′-pyridyldithio)propionamido)butane (DPDPB), bismaleimidohexane (BMH), aryl halide-containing compound (DFDNB), such as e.g. 1,5-difluoro-2,4-dinitrobenzene or 1,3-difluoro-4,6-dinitrobenzene, 4,4′-difluoro-3,3′-dinitrophenylsulfone (DFDNPS), bis-[2-(4-azidosalicylamido)ethyl]disulfide (BASED), formaldehyde, glutaraldehyde, 1,4-butanediol diglycidyl ether, adipic acid dihydrazide, carbohydrazide, o-toluidine, 3,3′-dimethylbenzidine, benzidine, α,α′-ρ-diaminodiphenyl, diiodo-p-xylene sulfonic acid, N,N′-ethylene-bis(iodoacetamide), or N,N′-hexamethylene-bis(iodoacetamide).

In some embodiments, the linker comprises a heterobifunctional linker. Exemplary heterobifunctional linker include, but are not limited to, amine-reactive and sulfhydryl cross-linkers such as N-succinimidyl 3-(2-pyridyldithio)propionate (sPDP), long-chain N-succinimidyl 3-(2-pyridyldithio)propionate (LC-sPDP), water-soluble-long-chain N-succinimidyl 3-(2-pyridyldithio) propionate (sulfo-LC-sPDP), succinimidyloxycarbonyl-a-methyl-a-(2-pyridyldithio)toluene (sMPT), sulfosuccinimidyl-6-[a-methyl-a-(2-pyridyldithio)toluamido]hexanoate (sulfo-LC-sMPT), succinimidyl-4-(N-maleimidomethyl)cyclohexane-1-carboxylate (sMCC), sulfosuccinimidyl-4-(N-maleimidomethyl)cyclohexane-1-carboxylate (sulfo-sMCC), m-maleimidobenzoyl-N-hydroxysuccinimide ester (MBs), m-maleimidobenzoyl-N-hydroxysulfosuccinimide ester (sulfo-MBs), N-succinimidyl(4-iodoacteyl)aminobenzoate (sIAB), sulfosuccinimidyl(4-iodoacteyl)aminobenzoate (sulfo-sIAB), succinimidyl-4-(p-maleimidophenyl)butyrate (sMPB), sulfosuccinimidyl-4-(p-maleimidophenyl)butyrate (sulfo-sMPB), N-(y-maleimidobutyryloxy)succinimide ester (GMBs), N-(y-maleimidobutyryloxy)sulfosuccinimide ester (sulfo-GMBs), succinimidyl 6-((iodoacetyl)amino)hexanoate (sIAX), succinimidyl 6-[6-(((iodoacetyl)amino)hexanoyl)amino]hexanoate (sIAXX), succinimidyl 4-(((iodoacetyl)amino)methyl)cyclohexane-1-carboxylate (sIAC), succinimidyl 6-((((4-iodoacetyl)amino)methyl)cyclohexane-1-carbonyl)amino) hexanoate (sIACX), p-nitrophenyl iodoacetate (NPIA), carbonyl-reactive and sulfhydryl-reactive cross-linkers such as 4-(4-N-maleimidophenyl)butyric acid hydrazide (MPBH), 4-(N-maleimidomethyl)cyclohexane-1-carboxyl-hydrazide-8 (M2C2H), 3-(2-pyridyldithio)propionyl hydrazide (PDPH), amine-reactive and photoreactive cross-linkers such as N-hydroxysuccinimidyl-4-azidosalicylic acid (NHs-AsA), N-hydroxysulfosuccinimidyl-4-azidosalicylic acid (sulfo-NHs-AsA), sulfosuccinimidyl-(4-azidosalicylamido)hexanoate (sulfo-NHs-LC-AsA), sulfosuccinimidyl-2-(p-azidosalicylamido)ethyl-1,3′-dithiopropionate (sAsD), N-hydroxysuccinimidyl-4-azidobenzoate (HsAB), N-hydroxysulfosuccinimidyl-4-azidobenzoate (sulfo-HsAB), N-succinimidyl-6-(4′-azido-2′-nitrophenylamino)hexanoate (sANPAH), sulfosuccinimidyl-6-(4′-azido-2′-nitrophenylamino)hexanoate (sulfo-sANPAH), N-5-azido-2-nitrobenzoyloxysuccinimide (ANB-NOs), sulfosuccinimidyl-2-(m-azido-o-nitrobenzamido)-ethyl-1,3′-dithiopropionate (sAND), N-succinimidyl-4(4-azidophenyl) 1,3′-dithiopropionate (sADP), N-sulfosuccinimidyl(4-azidophenyl)-1,3′-dithiopropionate (sulfo-sADP), sulfosuccinimidyl 4-(p-azidophenyl)butyrate (sulfo-sAPB), sulfosuccinimidyl 2-(7-azido-4-methylcoumarin-3-acetamide)ethyl-1,3′-dithiopropionate (sAED), sulfosuccinimidyl 7-azido-4-methylcoumain-3-acetate (sulfo-sAMCA), p-nitrophenyl diazopyruvate (pNPDP), p-nitrophenyl-2-diazo-3,3,3-trifluoropropionate (PNP-DTP), sulfhydryl-reactive and photoreactive cross-linkers such as 1-(p-Azidosalicylamido)-4-(iodoacetamido)butane (AsIB), N-[4-(p-azidosalicylamido)butyl]-3′-(2′-pyridyldithio)propionamide (APDP), benzophenone-4-iodoacetamide, benzophenone-4-maleimide carbonyl-reactive and photoreactive cross-linkers such as p-azidobenzoyl hydrazide (ABH), carboxylate-reactive and photoreactive cross-linkers such as 4-(p-azidosalicylamido)butylamine (AsBA), and arginine-reactive and photoreactive cross-linkers such as p-azidophenyl glyoxal (APG).

In some embodiments, the linker comprises a reactive functional group. In some embodiments, the reactive functional group comprises a nucleophilic group that is reactive to an electrophilic group present on a binding moiety. Exemplary electrophilic groups include carbonyl groups—such as aldehyde, ketone, carboxylic acid, ester, amide, enone, acyl halide or acid anhydride. In some embodiments, the reactive functional group is aldehyde. Exemplary nucleophilic groups include hydrazide, oxime, amino, hydrazine, thiosemicarbazone, hydrazine carboxylate, and arylhydrazide.

In some embodiments, the linker comprises a maleimide group. In some embodiments, the maleimide group is also referred to as a maleimide spacer. In some embodiments, the maleimide group further encompasses a caproic acid, forming maleimidocaproyl (me). In some embodiments, the linker comprises maleimidocaproyl (me). In some embodiments, the linker is maleimidocaproyl (me). In other instances, the maleimide group comprises a maleimidomethyl group, such as succinimidyl-4-(N-maleimidomethyl)cyclohexane-1-carboxylate (sMCC) or sulfosuccinimidyl-4-(N-maleimidomethyl)cyclohexane-1-carboxylate (sulfo-sMCC) described above.

In some embodiments, the maleimide group is a self-stabilizing maleimide. In some embodiments, the self-stabilizing maleimide utilizes diaminopropionic acid (DPR) to incorporate a basic amino group adjacent to the maleimide to provide intramolecular catalysis of thiosuccinimide ring hydrolysis, thereby eliminating maleimide from undergoing an elimination reaction through a retro-Michael reaction. In some embodiments, the self-stabilizing maleimide is a maleimide group described in Lyon, et al, “Self-hydrolyzing maleimides improve the stability and pharmacological properties of antibody-drug conjugates,” Nat. Biotechnol. 32(10): 1059-1062 (2014). In some embodiments, the linker comprises a self-stabilizing maleimide. In some embodiments, the linker is a self-stabilizing maleimide.

In some embodiments, the linker comprises a peptide moiety. In some embodiments, the peptide moiety comprises at least 2, 3, 4, 5, 6, 7, 8, or more amino acid residues. In some embodiments, the peptide moiety is a cleavable peptide moiety (e.g., either enzymatically or chemically). In some embodiments, the peptide moiety is a non-cleavable peptide moiety. In some embodiments, the peptide moiety comprises Val-Cit (valine-citrulline), Gly-Gly-Phe-Gly, Phe-Lys, Val-Lys, Gly-Phe-Lys, Phe-Phe-Lys, Ala-Lys, Val-Arg, Phe-Cit, Phe-Arg, Leu-Cit, Ile-Cit, Tφ-Cit, Phe-Ala, Ala-Leu-Ala-Leu, or Gly-Phe-Leu-Gly. In some embodiments, the linker comprises a peptide moiety such as: Val-Cit (valine-citrulline), Gly-Gly-Phe-Gly, Phe-Lys, Val-Lys, Gly-Phe-Lys, Phe-Phe-Lys, Ala-Lys, Val-Arg, Phe-Cit, Phe-Arg, Leu-Cit, Ile-Cit, Trp-Cit, Phe-Ala, Ala-Leu-Ala-Leu, or Gly-Phe-Leu-Gly. In some embodiments, the linker comprises Val-Cit. In some embodiments, the linker is Val-Cit.

In some embodiments, the linker comprises a benzoic acid group, or its derivatives thereof. In some embodiments, the benzoic acid group or its derivatives thereof comprise paraaminobenzoic acid (PABA). In some embodiments, the benzoic acid group or its derivatives thereof comprise gamma-aminobutyric acid (GABA).

In some embodiments, the linker comprises one or more of a maleimide group, a peptide moiety, and/or a benzoic acid group, in any combination. In some embodiments, the linker comprises a combination of a maleimide group, a peptide moiety, and/or a benzoic acid group. In some embodiments, the maleimide group is maleimidocaproyl (me). In some embodiments, the peptide group is val-cit. In some embodiments, the benzoic acid group is PABA. In some embodiments, the linker comprises a mc-val-cit group. In some embodiments, the linker comprises a val-cit-PABA group. In additional cases, the linker comprises a mc-val-cit-PABA group.

In some embodiments, the linker is a self-immolative linker or a self-elimination linker. In some embodiments, the linker is a self-immolative linker. In other cases, the linker is a self-elimination linker (e.g., a cyclization self-elimination linker). In some embodiments, the linker comprises a linker described in U.S. Pat. No. 9,089,614 or PCT Publication No. WO2015038426, each of which is incorporated herein by reference in its entirety.

In some embodiments, the linker is a dendritic type linker. In some embodiments, the dendritic type linker comprises a branching, multifunctional linker moiety. In some embodiments, the dendritic type linker is used to increase the molar ratio of polynucleotide B to the binding moiety A. In some embodiments, the dendritic type linker comprises PAMAM dendrimers.

In some embodiments, the linker is a traceless linker or a linker in which after cleavage does not leave behind a linker moiety (e.g., an atom or a linker group) to a polynucleotide or a targeting molecule. Exemplary traceless linkers include, but are not limited to, germanium linkers, silicium linkers, sulfur linkers, selenium linkers, nitrogen linkers, phosphorus linkers, boron linkers, chromium linkers, or phenylhydrazide linker. In some embodiments, the linker is a traceless aryl-triazene linker as described in Hejesen, et al., “A traceless aryl-triazene linker for DNA-directed chemistry,” Org Biomol Chem 11(15): 2493-2497 (2013). In some embodiments, the linker is a traceless linker described in Blaney, et al., ‘Traceless solid-phase organic synthesis,” Chem. Rev. 102: 2607-2024 (2002). In some embodiments, a linker is a traceless linker as described in U.S. Pat. No. 6,821,783, incorporated herein by reference in its entirety.

In some embodiments, the linker comprises a functional group that exerts steric hinderance at the site of bonding between the linker and a conjugating moiety (e.g., a polynucleotide or a targeting molecule disclosed herein). In some embodiments, the steric hinderance is a steric hindrance around a disulfide bond. Exemplary linkers that exhibit steric hinderance comprises a heterobifunctional linker, such as a heterobifunctional linker described above. In some embodiments, a linker that exhibits steric hinderance comprises SMCC and SPDB.

In some embodiments, the linker is an acid cleavable linker. In some embodiments, the acid cleavable linker comprises a hydrazone linkage, which is susceptible to hydrolytic cleavage. In some embodiments, the acid cleavable linker comprises a thiomaleamic acid linker. In some embodiments, the acid cleavable linker is a thiomaleamic acid linker as described in Castaneda, et al, “Acid-cleavable thiomaleamic acid linker for homogeneous antibody-drug conjugation,” Chem. Commun. 49: 8187-8189 (2013).

In some embodiments, the linker is a linker described in U.S. Pat. Nos. 6,884,869; 7,498,298; 8,288,352; 8,609,105; or 8,697,688; U.S. Patent Publication Nos. 2014/0127239; 2013/028919; 2014/286970; 2013/0309256; 2015/037360; or 2014/0294851; or PCT Publication Nos. WO2015057699; WO2014080251; WO2014197854; WO2014145090; or WO2014177042, each of which is incorporated herein by reference in its entirety.

In some embodiments, the linker is conjugated to a lysine residue, a cysteine residue, a histidine residue, or a non-natural amino acid residue in the targeting molecule. In some embodiments, the linker is conjugated to a lysine residue in the targeting molecule. In some embodiments, the linker is conjugated to a cysteine residue in the targeting molecule. In some embodiments, the linker is conjugated to a histidine residue in the targeting molecule. In some embodiments, the linker is conjugated to a non-natural amino acid residue in the targeting molecule.

In some embodiments, the linker is conjugated to the targeting molecule by a chemical conjugation or an enzymatic conjugation. In some embodiments, the linker is conjugated to the targeting molecule by a chemical conjugation. The chemical conjugation may comprise acylation and click chemistry. In some embodiments, the linker is conjugated to the targeting molecule by an enzymatic conjugation. The enzymatic conjugation may be via a sortase or a transferase enzyme.

In some embodiments, the polynucleotide is conjugated to the targeting molecule by a chemical ligation process. In some embodiments, the polynucleotide is conjugated to the targeting molecule by a native ligation. In some embodiments, the conjugation is as described in: Dawson, et al. “Synthesis of proteins by native chemical ligation,” Science 1994, 266, 776-779; Dawson, et al. “Modulation of Reactivity in Native Chemical Ligation through the Use of Thiol Additives,” J. Am. Chem. Soc. 1997, 119, 4325-4329; Hackeng, et al. “Protein synthesis by native chemical ligation: Expanded scope by using straightforward methodology,” Proc. Natl. Acad. Sci. USA 1999, 96, 10068-10073; or Wu, et al. “Building complex glycopeptides: Development of a cysteine-free native chemical ligation protocol,” Angew. Chem. Int. Ed. 2006, 45, 4116-4125. In some embodiments, the conjugation is as described in U.S. Pat. No. 8,936,910. In some embodiments, the polynucleotide is conjugated to the targeting molecule either site-specifically or non-specifically via native ligation chemistry.

In some embodiments, the polynucleotide is conjugated to the targeting molecule by a site-directed method utilizing a “traceless” coupling technology (Philochem). In some embodiments, the “traceless” coupling technology utilizes an N-terminal 1,2-aminothiol group on the targeting molecule which is then conjugate with a polynucleotide containing an aldehyde group. (see Casi et al., “Site-specific traceless coupling of potent cytotoxic drugs to recombinant antibodies for pharmacodelivery,” JACS 134(13): 5887-5892 (2012))

In some embodiments, the polynucleotide is conjugated to the targeting molecule by a site-directed method utilizing an unnatural amino acid incorporated into the targeting molecule. In some embodiments, the unnatural amino acid comprises p-acetylphenylalanine (pAcPhe). In some embodiments, the keto group of pAcPhe is selectively coupled to an alkoxy-amine derivatized conjugating moiety to form an oxime bond. (see Axup et al., “Synthesis of site-specific antibody-drug conjugates using unnatural amino acids,” PNAS 109(40): 16101-16106 (2012)).

In some embodiments, the polynucleotide is conjugated to the targeting molecule by a site-directed method utilizing an enzyme-catalyzed process. In some embodiments, the site-directed method utilizes SMARTag™ technology (Redwood). In some embodiments, the SMARTag™ technology comprises generation of a formylglycine (FGly) residue from cysteine by formylglycine-generating enzyme (FGE) through an oxidation process under the presence of an aldehyde tag and the subsequent conjugation of FGly to an alkylhydraine-functionalized polynucleotide via hydrazino-Pictet-Spengler (HIPS) ligation. (see Wu et al., “Site-specific chemical modification of recombinant proteins produced in mammalian cells by using the genetically encoded aldehyde tag,” PNAS 106(9): 3000-3005 (2009); Agarwal, et al., “A Pictet-Spengler ligation for protein chemical modification,” PNAS 110(1): 46-51 (2013))

In some embodiments, the enzyme-catalyzed process comprises microbial transglutaminase (mTG). In some embodiments, the polynucleotide is conjugated to the targeting molecule utilizing a microbial transglutaminase catalyzed process. In some embodiments, mTG catalyzes the formation of a covalent bond between the amide side chain of a glutamine within the recognition sequence and a primary amine of a functionalized polynucleotide. In some embodiments, mTG is produced from Streptomyces mobarensis. (see Strop et al., “Location matters: site of conjugation modulates stability and pharmacokinetics of antibody drug conjugates,” Chemistry and Biology 20(2) 161-167 (2013))

In some embodiments, the polynucleotide is conjugated to the targeting molecule by a method as described in PCT Publication No. WO2014/140317 (incorporated herein by reference in its entirety), which utilizes a sequence-specific transpeptidase. In some embodiments, the polynucleotide is conjugated to the targeting molecule by a method as described in U.S. Patent Publication Nos. 2015/0105539 and 2015/0105540, each of which is incorporated herein by reference in its entirety.

In some embodiments, each targeting molecule is conjugated to between one and eight polynucleotide molecules (i.e. a Drug:Antibody Ratio (DAR) between 1 and 8). In some embodiments, each targeting molecule is conjugated to one polynucleotide molecule (DAR of 1). In some embodiments, each targeting molecule is conjugated to two polynucleotide molecules (DAR of 2). In some embodiments, each targeting molecule is conjugated to three polynucleotide molecules (DAR of 3). In some embodiments, each targeting molecule is conjugated to four polynucleotide molecules (DAR of 4). In some embodiments, each targeting molecule is conjugated to five polynucleotide molecules (DAR of 5). In some embodiments, each targeting molecule is conjugated to six polynucleotide molecules (DAR of 6). In some embodiments, each targeting molecule is conjugated to seven polynucleotide molecules (DAR of 7). In some embodiments, each targeting molecule is conjugated to eight polynucleotide molecules (DAR of 8).

In some embodiments, the polynucleotide-conjugated targeting molecule has a molecular weight greater than about 30 kDa. In some embodiments, the polynucleotide-conjugated targeting molecule has a molecular weight greater than about 40 kDa. The polynucleotide-conjugated targeting molecule may have a molecular weight greater than about 50 kDa. In some embodiments, the polynucleotide-conjugated targeting molecule has a molecular weight greater than about 60 kDa. In some embodiments, the polynucleotide-conjugated targeting molecule has a molecular weight no greater than about 7,500 kDa.

In some embodiments, the polynucleotide-conjugated targeting molecule has a molecular weight greater than 30 kDa. In some embodiments, the polynucleotide-conjugated targeting molecule has a molecular weight greater than 40 kDa. The polynucleotide-conjugated targeting molecule may have a molecular weight greater than 50 kDa. In some embodiments, the polynucleotide-conjugated targeting molecule has a molecular weight greater than 60 kDa. In some embodiments, the polynucleotide-conjugated targeting molecule has a molecular weight no greater than 7,500 kDa.

The present disclosure further relates to a method of treating a pathology in an individual, comprising administering to the individual a therapeutic targeting agent that specifically binds a targeted protein expressed on muscle tissue cell surface. “Pathology” and “disease” are used interchangeably herein. The pathology may be a genetic disease. Another aspect of this disclosure provides methods for treating genetic diseases in a subject in need thereof. In some embodiments, the method comprises administering to the subject a therapeutically effective amount of any of the compositions for delivering polynucleotides disclosed herein. In some embodiments, the method comprises administering to the subject a therapeutically effective amount of any of the polynucleotide conjugates disclosed herein.

A genetic disease, as disclosed herein, may be a cancer, a neurological disorder, a fibrosis disease, a scarring disease, an autoimmune disease, or an inherited genetic disorder.

In some embodiments, the genetic disease is a neurological disorder. In some embodiments, the neurological disorder is selected from the group consisting of Acquired Epileptiform Aphasia, Acute Disseminated Encephalomyelitis, Adrenoleukodystrophy, Agenesis of the corpus callosum, Agnosia, Aicardi syndrome, Alexander disease, Alpers' disease, Alternating hemiplegia, Alzheimer's disease, Amyotrophic lateral sclerosis (see Motor Neuron Disease), Anencephaly, Angelman syndrome, Angiomatosis, Anoxia, Aphasia, Apraxia, Arachnoid cysts, Arachnoiditis, Arnold-Chiari malformation, Arteriovenous mal-formation, Asperger's syndrome, Ataxia Telangiectasia, Attention Deficit Hyperactivity Disorder, Autism, Auditory processing disorder, Autonomic Dysfunction, Back Pain, Batten disease, Bechet's disease, Bell's palsy, Benign Essential Blepharospasm, Benign Focal Amyotrophy, Benign Intracranial Hypertension, Bilateral frontoparietal polymicrogyria, Binswanger's disease, Blepharospasm, Bloch-Sulzberger syndrome, Brachial plexus injury, Brain abscess, Brain damage, Brain injury, Brain tumor, Brown-Sequard syndrome, Canavan disease, Carpal tunnel syndrome (CTS), Causalgia, Central pain syndrome, Central pontine myelinolysis, Centronuclear myopathy, Cephalic disorder, Cerebral aneurysm, Cerebral arteriosclerosis, Cerebral atrophy, Cerebral gigantism, Cerebral palsy, Charcot-Marie-Tooth disease, Chiari malformation, Chorea, Chronic inflammatory de-myelinating polyneuropathy (CIDP), Chronic pain, Chronic regional pain syndrome, Coffin Lowry syndrome, Coma, including Persistent Vegetative State, Congenital facial diplegia, Corticobasal degeneration, Cranial arteritis, Craniosynostosis, Creutzfeldt-Jakob disease, Cumulative trauma disorders, Cushing's syndrome, Cytomegalic inclusion body disease (CIBD), Cytomegalovirus Infection, Dandy-Walker syndrome, Dawson disease, De Morsier's syndrome, Dejerine-Klumpke palsy, Dejerine-Sottas disease, Delayed sleep phase syndrome, Dementia, Dermatomyositis, Neurological Dyspraxia, Diabetic neuropathy, Diffuse sclerosis, Dysautonomia, Dyscalculia, Dysgraphia, Dyslexia, Dystonia, Early infantile epileptic encephalopathy, Empty sella syndrome, Encephalitis, Encephalocele, Encephalotrigeminal angiomatosis, Encopresis, Epilepsy, Erb's palsy, Erythromelalgia, Essential tremor, Fabry's disease, Fahr's syndrome, Fainting, Familial spastic paralysis, Febrile seizures, Fisher syndrome, Friedreich's ataxia, FART Syndrome, Gaucher's disease, Gerstmann's syndrome, Giant cell arteritis, Giant cell inclusion disease, Globoid cell Leukodystrophy, Gray matter heterotopia, Guillain-Barre syndrome, HTLV-1 associated myelopathy, Hallervorden-Spatz disease, Head injury, Headache, Hemifacial Spasm, Hereditary Spastic Paraplegia, Heredopathia atactica polyneuritiformis, Herpes zoster oticus, Herpes zoster, Hirayama syndrome, Holoprosencephaly, Huntington's disease, Hydranencephaly, Hydrocephalus, Hypercortisolism, hypertrophic cardiomyopathy, Hypoxia, Immune-Mediated encephalomyelitis, Inclusion body myositis, Incontinentia pigmenti, Infantile phytanic acid storage disease, Infantile Refsum disease, Infantile spasms, Inflammatory myopathy, Intracranial cyst, Intracranial hypertension, Joubert syndrome, Kearns-Sayre syndrome, Kennedy disease, Kinsbourne syndrome, Klippel Feil syndrome, Krabbe disease, Kugelberg-Welander disease, Kuru, Lafora disease, Lambert-Eaton myasthenic syndrome, Landau-Kleffner syndrome, Lateral medullary (Wallenberg) syndrome, Learning disabilities, Leigh's disease, Lennox-Gastaut syndrome, Lesch-Nyhan syndrome, Leukodystrophy, Lewy body dementia, Lissencephaly, Locked-In syndrome, Lou Gehrig's disease, Lumbar disc disease, Lyme disease—Neurological Sequelae, Macha-do-Joseph disease (Spinocerebellar ataxia type 3), Macrencephaly, Maple Syrup Urine Disease, Marfan syndrome, Megalencephaly, Melkersson-Rosenthal syndrome, Menieres disease, Meningitis, Menkes disease, Metachromatic leukodystrophy, Microcephaly, Migraine, Miller Fisher syndrome, Mini-Strokes, Mitochondrial Myopathies, Mobius syndrome, Monomelic amyotrophy, Motor Neuron Disease, Motor skills disorder, Moyamoya disease, Mucopolysaccharidoses, Multi-Infarct Dementia, Multifocal motor neuropathy, Multiple sclerosis, Multiple system atrophy, Muscular dystrophy, Myalgic encephalomyelitis, Myasthenia gravis, Myelinoclastic diffuse sclerosis, Myoclonic Encephalopathy of infants, Myoclonus, Myopathy, Myotubular myopathy, Myotonia congenita, Narcolepsy, Neurofibromatosis, Neuroleptic malignant syndrome, Neurological manifestations of AIDS, Neurological sequelae of lupus, Neuromyotonia, Neuronal ceroid lipofuscinosis, Neuronal migration disorders, Niemann-Pick disease, Non 24-hour sleep-wake syndrome, Nonverbal learning disorder, O'Sullivan-McLeod syndrome, Occipital Neuralgia, Occult Spinal Dysraphism Sequence, Ohtahara syndrome, Olivopontocerebellar atrophy, Opsoclonus myoclonus syndrome, Optic neuritis, Orthostatic Hypotension, Overuse syndrome, Palinopsia, Paresthesia, Parkinson's disease, Paramyotonia Congenita, Paraneoplastic diseases, Paroxysmal attacks, Parry-Romberg syndrome, Rombergs Syndrome, Pelizaeus-Merzbacher disease, Periodic Paralyses, Peripheral neuropathy, Persistent Vegetative State, Pervasive neurological disorders, Photic sneeze reflex, Phytanic Acid Storage disease, Pick's disease, Pinched Nerve, Pituitary Tumors, PMG, Polio, Polymicrogyria, Polymyositis, Porencephaly, Post-Polio syndrome, Postherpetic Neuralgia (PHN), Postinfectious Encephalomyelitis, Postural Hypotension, Prader-Willi syndrome, Primary Lateral Sclerosis, Prion diseases, Progressive Hemifacial Atrophy also known as Rombergs Syndrome, Progressive multifocal leukoencephalopathy, Progressive Sclerosing Poliodystrophy, Progressive Supranuclear Palsy, Pseudotumor cerebri, Ramsay-Hunt syndrome (Type I and Type II), Rasmussen's encephalitis, Reflex sympathetic dystrophy syndrome, Refsum disease, Repetitive motion disorders, Repetitive stress injury, Restless legs syndrome, Retrovirus-associated myelopathy, Rett syndrome, Reye's syndrome, Rombergs Syndrome, Rabies, Saint Vitus dance, Sandhoff disease, Schytsophrenia, Schilder's disease, Schizencephaly, Sensory Integration Dysfunction, Septooptic dysplasia, Shaken baby syndrome, Shingles, Shy-Drager syndrome, Sjogren's syndrome, Sleep apnea, Sleeping sickness, Snatiation, Sotos syndrome, Spasticity, Spina bifida, Spinal cord injury, Spinal cord tumors, Spinal muscular atrophy, Spinal stenosis, Steele-Richardson-Olszewski syndrome, see Progressive Supranuclear Palsy, Spinocerebellar ataxia, Stiff person syndrome, Stroke, Sturge-Weber syndrome, Subacute sclerosing panencephalitis, Subcortical arteriosclerotic encephalopathy, Superficial siderosis, Sydenham's chorea, Syncope, Synesthesia, Syringomyelia, Tardive dyskinesia, Tay-Sachs disease, Temporal arteritis, Tethered spinal cord syndrome, Thomsen disease, Thoracic outlet syndrome, Tic Douloureux, Todd's paralysis, Tourette syndrome, Transient ischemic attack, Transmissible spongiform encephalopathies, Transverse myelitis, Traumatic brain injury, Tremor, Trigeminal neuralgia, Tropical spastic paraparesis, Trypanosomiasis, Tuberous sclerosis, Vasculitis including temporal arteritis, Von Hippel-Lindau disease (VHL), Viliuisk Encephalomyelitis (VE), Wallenberg's syndrome, Werdnig-Hoffman disease, West syndrome, Whiplash, Williams syndrome, Wilson's disease, X-Linked Spinal and Bulbar Muscular Atrophy, and Zellweger syndrome. In some embodiments, the neurological disorder is a movement disorder, for example multiple system atrophy (MSA).

In some embodiments, the genetic disease is an autoimmune disease. In some embodiments, the autoimmune disease is selected from the group consisting of acute disseminated encephalomyelitis (ADEM), acute necrotizing hemorrhagic leukoencephalitis, Addison's disease, agammaglobulinemia, allergic asthma, allergic rhinitis, alopecia areata, amyloidosis, ankylosing spondylitis, anti-GBM/anti-TBM nephritis, antiphospholipid syndrome (APS), autoimmune aplastic anemia, autoimmune dysautonomia, autoimmune hepatitis, autoimmune hyperlipidemia, autoimmune immunodeficiency, autoimmune inner ear disease (AIED), autoimmune myocarditis, autoimmune pancreatitis, autoimmune retinopathy, autoimmune thrombocytopenic purpura (ATP), autoimmune thyroid disease, axonal & neuronal neuropathies, Balo disease, Bechet's disease, bullous pemphigoid, cardiomyopathy, Castlemen disease, celiac sprue (non-tropical), Chagas disease, chronic fatigue syndrome, chronic inflammatory demyelinating polyneuropathy (CIDP), chronic recurrent multifocal ostomyelitis (CRMO), Churg-Strauss syndrome, cicatricial pemphigoid/benign mucosal pemphigoid, Crohn's disease, Cogan's syndrome, cold agglutinin disease, congenital heart block, coxsackie myocarditis, CREST disease, essential mixed cryoglobulinemia, demyelinating neuropathies, dermatomyositis, Devic's disease (neuromyelitis optica), discoid lupus, Dressler's syndrome, endometriosis, eosinophillic fasciitis, erythema nodosum, experimental allergic encephalomyelitis, Evan's syndrome, fibromyalgia, fibrosing alveolitis, giant cell arteritis (temporal arteritis), glomerulonephritis, Goodpasture's syndrome, Grave's disease, Guillain-Barre syndrome, Hashimoto's encephalitis, Hashimoto's thyroiditis, hemolytic anemia, Henock-Schoniein purpura, herpes gestationis, hypogammaglobulinemia, idiopathic thrombocytopenic purpura (ITP), IgA nephropathy, immunoregulatory lipoproteins, inclusion body myositis, insulin-dependent diabetes (type 1), interstitial cystitis, juvenile arthritis, juvenile diabetes, Kawasaki syndrome, Lambert-Eaton syndrome, leukocytoclastic vasculitis, lichen planus, lichen sclerosis, ligneous conjunctivitis, linear IgA disease (LAD), Lupus (SLE), Lyme disease, Meniere's disease, microscopic polyangitis, mixed connective tissue disease (MCTD), Mooren's ulcer, Mucha-Habermann disease, multiple sclerosis, myasthenia gravis, myositis, narcolepsy, neuromyelitis optica (Devic's), neutropenia, ocular cicatricial pemphigoid, optic neuritis, palindromic rheumatism, PANDAS (Pediatric Autoimmune Neuropsychiatric Disorders Associated with Streptococcus), paraneoplastic cerebellar degeneration, paroxysmal nocturnal hemoglobinuria (PNH), Parry Romberg syndrome, Parsonnage-Turner syndrome, pars plantis (peripheral uveitis), pemphigus, peripheral neuropathy, perivenous encephalomyelitis, pernicious anemia, POEMS syndrome, polyarteritis nodosa, type I, II & III autoimmune polyglandular syndromes, polymyalgia rheumatic, polymyositis, postmyocardial infarction syndrome, postpericardiotomy syndrome, progesterone dermatitis, primary biliary cirrhosis, primary sclerosing cholangitis, psoriasis, psoriatic arthritis, idiopathic pulmonary fibrosis, pyoderma gangrenosum, pure red cell aplasis, Raynaud's phenomena, reflex sympathetic dystrophy, Reiter's syndrome, relapsing polychondritis, restless legs syndrome, retroperitoneal fibrosis, rheumatic fever, rheumatoid arthritis, sarcoidosis, Schmidt syndrome, scleritis, scleroderma, Slogren's syndrome, sperm and testicular autoimmunity, stiff person syndrome, subacute bacterial endocarditis (SBE), sympathetic ophthalmia, Takayasu's arteritis, temporal arteritis/giant cell arteries, thrombocytopenic purpura (TPP), Tolosa-Hunt syndrome, transverse myelitis, ulcerative colitis, undifferentiated connective tissue disease (UCTD), uveitis, vasculitis, vesiculobullous dermatosis, vitiligo or Wegener's granulomatosis or, chronic active hepatitis, primary biliary cirrhosis, cadilated cardiomyopathy, myocarditis, autoimmune polyendocrine syndrome type I (APS-I), cystic fibrosis vasculitides, acquired hypoparathyroidism, coronary artery disease, pemphigus foliaceus, pemphigus vulgaris, Rasmussen encephalitis, autoimmune gastritis, insulin hypoglycemic syndrome (Hirata disease), Type B insulin resistance, acanthosis, systemic lupus erythematosus (SLE), pernicious anemia, treatment-resistant Lyme arthritis, polyneuropathy, demyelinating diseases, atopic dermatitis, autoimmune hypothyroidism, vitiligo, thyroid associated ophthalmopathy, autoimmune coeliac disease, ACTH deficiency, dermatomyositis, Sjogren syndrome, systemic sclerosis, progressive systemic sclerosis, morphea, primary antiphospholipid syndrome, chronic idiopathic urticaria, connective tissue syndromes, necrotizing and crescentic glomerulonephritis (NCGN), systemic vasculitis, Raynaud syndrome, chronic liver disease, visceral leishmaniasis, autoimmune C1 deficiency, membrane proliferative glomerulonephritis (MPGN), prolonged coagulation time, immunodeficiency, atherosclerosis, neuronopathy, paraneoplastic pemphigus, paraneoplastic stiff man syndrome, paraneoplastic encephalomyelitis, subacute autonomic neuropathy, cancer-associated retinopathy, paraneoplastic opsoclonus myoclonus ataxia, lower motor neuron syndrome and Lambert-Eaton myasthenic syndrome.

In some embodiment, the genetic disease may be selected from the group consisting of AIDS, anthrax, botulism, brucellosis, chancroid, chlamydial infection, cholera, coccidioidomycosis, cryptosporidiosis, cyclosporiasis, dipheheria, ehrlichiosis, arboviral encephalitis, enterohemorrhagic Escherichia coli, giardiasis, gonorrhea, dengue fever, haemophilus influenza, Hansen's disease (Leprosy), hantavirus pulmonary syndrome, hemolytic uremic syndrome, hepatitis A, hepatitis B, hepatitis C, human immunodeficiency virus, legionellosis, listeriosis, Lyme disease, malaria, measles. Meningococcal disease, mumps, pertussis (whooping cough), plague, paralytic poliomyelitis, psittacosis, Q fever, rabies, rocky mountain spotted fever, rubella, congenital rubella syndrome, shigellosis, smallpox, streptococcal disease (invasive group A), streptococcal toxic shock syndrome, Streptococcus pneumonia, syphilis, tetanus, toxic shock syndrome, trichinosis, tuberculosis, tularemia, typhoid fever, vancomycin intermediate resistant Staphylocossus aureus, varicella, yellow fever, variant Creutzfeldt-Jakob disease (vCJD), Ebola hemorrhagic fever, Echinococcosis, Hendra virus infection, human monkeypox, influenza A, influenza B, H5N1, lassa fever, Marburg hemorrhagic fever, Nipah virus, O'nyong fever, Rift valley fever, Herpes, HIV, HCV genotype 1, HCV genotype 2, HCV genotype 3, HCV genotype 4, HCV genotype 5, HCV genotype 6, SARS-CoV-2 (COVID-19), SARS-CoV (SARS), MERS-CoV (MERS), 229E coronavirus, NL63 coronavirus, OC43 coronavirus, CoV—HKU1(HKU1), alpha coronavirus, beta coronavirus, Venezuelan equine encephalitis and West Nile virus.

In some embodiments, the genetic disease is a fibrosis disease, a scarring disease or both. In some embodiments, the fibrosis disease or the scarring disease is selected from the group consisting of pulmonary fibrosis, cystic fibrosis, idiopathic pulmonary fibrosis, radiation induced fibrosis, myocardial fibrosis, bridging fibrosis, cirrhosis, gliosis, arterial stiffness, arthrofibrosis, Chron's disease, Dupuytren's contracture, keloid, mediastinal fibrosis, myelofibrosis, Peyronie's disease, nephrogenic systemic fibrosis, progressive massive fibrosis, retroperitoneal fibrosis, scleroderma/systemic sclerosis, and adhesive capsulitis.

In some embodiments, the genetic disease is a muscle related cancer, such as a sarcoma.

In some embodiments, the genetic disease is an inherited genetic disorder caused by abnormalities in genes or chromosomes. Inherited genetic disorders can be grouped into two categories: single gene disorders and multifactorial and polygenic (complex) disorders. A single gene disorder may be the result of a single mutated gene. Inheriting a single gene disorder can include but not be limited to autosomal dominant, autosomal recessive, X-linked dominant, X-linked recessive, Y-linked and mitochondrial inheritance. In some embodiments, one mutated copy of the gene is necessary for a person to be affected by an autosomal dominant disorder. Examples of autosomal dominant type of disorder can include but are not limited to Huntington's disease, Neurofibromatosis 1, Marfan Syndrome, Hereditary nonpolyposis colorectal cancer, or Hereditary multiple exostoses. In autosomal recessive disorders, two copies of the gene must be mutated for a subject to be affected by the autosomal recessive disorder. Examples of this type of disorder can include but may not be limited to cystic fibrosis, sickle-cell disease (also partial sickle-cell disease), Tay-Sachs disease, Niemann-Pick disease, or spinal muscular atrophy. X-linked dominant disorders are caused by mutations in genes on the X chromosome such as X-linked hypophosphatemic rickets. Some X-linked dominant conditions such as Rett syndrome, Incontinentia Pigmenti type 2 and Aicardi Syndrome can be fatal. X-linked recessive disorders are also caused by mutations in genes on the X chromosome. Examples of this type of disorder can include but are not limited to Hemophilia A, Duchenne muscular dystrophy, red-green color blindness, muscular dystrophy and Androgenetic alopecia. Y-linked disorders are caused by mutations on the Y chromosome. Examples can include but are not limited to Male Infertility and hypertrichosis pinnae. The genetic disorder of mitochondrial inheritance, also known as maternal inheritance, can apply to genes in mitochondrial DNA such as in Leber's Hereditary Optic Neuropathy.

Inherited genetic disorders may also be complex, multifactorial or polygenic. Polygenic inherited genetic disorders may be associated with the effects of multiple genes in combination with lifestyle and environmental factors. Although complex genetic disorders can cluster in families, they do not have a clear-cut pattern of inheritance. Multifactorial or polygenic disorders include, but are not limited to, heart disease, diabetes, asthma, autism, autoimmune diseases such as multiple sclerosis, cancers, ciliopathies, cleft palate, hypertension, inflammatory bowel disease, mental retardation or obesity.

Other exemplary inherited genetic disorders include but may not be limited to 1p36 deletion syndrome, 21-hydroxylase deficiency, 22q11.2 deletion syndrome, aceruloplasminemia, achondrogenesis, type II, achondroplasia, acute intermittent porphyria, adenylosuccinate lyase deficiency, Adrenoleukodystrophy, Alexander disease, alkaptonuria, alpha-1 antitrypsin deficiency, Alstrom syndrome, Alzheimer's disease (type 1, 2, 3, and 4), Amelogenesis Imperfecta, amyotrophic lateral sclerosis, Amyotrophic lateral sclerosis type 2, Amyotrophic lateral sclerosis type 4, amyotrophic lateral sclerosis type 4, androgen insensitivity syndrome, Anemia, Angelman syndrome, Apert syndrome, ataxia-telangiectasia, Beare-Stevenson cutis gyrata syndrome, Benjamin syndrome, beta thalassemia, biotimidase deficiency, Birt-Hogg-Dube syndrome, bladder cancer, Bloom syndrome, Bone diseases, breast cancer, Camptomelic dysplasia, Canavan disease, Cancer, Celiac Disease, Chronic Granulomatous Disorder (CGD), Charcot-Marie-Tooth disease, Charcot-Marie-Tooth disease Type 1, Charcot-Marie-Tooth disease Type 4, Charcot-Marie-Tooth disease Type 2, Charcot-Marie-Tooth disease Type 4, Cockayne syndrome, Coffin-Lowry syndrome, collagenopathy types II and XI, Colorectal Cancer, Congenital absence of the vas deferens, congenital bilateral absence of vas deferens, congenital diabetes, congenital erythropoietic porphyria, Congenital heart disease, congenital hypothyroidism, Connective tissue disease, Cowden syndrome, Cri du chat syndrome, Crohn's disease, fibrostenosing, Crouzon syndrome, Crouzonodermoskeletal syndrome, cystic fibrosis, De Grouchy Syndrome, Degenerative nerve diseases, Dent's disease, developmental disabilities, DiGeorge syndrome, Distal spinal muscular atrophy type V, Down syndrome, Dwarfism, Ehlers-Danlos syndrome, Ehlers-Danlos syndrome arthrochalasia type, Ehlers-Danlos syndrome classical type, Ehlers-Danlos syndrome dermatosparaxis type, Ehlers-Danlos syndrome kyphoscoliosis type, vascular type, erythropoietic protoporphyria, Fabry's disease, Facial injuries and disorders, factor V Leiden thrombophilia, familial adenomatous polyposis, familial dysautonomia, fanconi anemia, FG syndrome, fragile X syndrome, Friedreich ataxia, Friedreich's ataxia, G6PD deficiency, galactosemia, Gaucher's disease (type 1, 2, and 3), Genetic brain disorders, Glycine encephalopathy, Haemochromatosis type 2, Haemochromatosis type 4, Harlequin Ichthyosis, Head and brain malformations, Hearing disorders and deafness, Hearing problems in children, hemochromatosis (neonatal, type 2 and type 3), hemophilia, hepatoerythropoietic porphyria, hereditary coproporphyria, Hereditary Multiple Exostoses, hereditary neuropathy with liability to pressure palsies, hereditary non-polyposis colorectal cancer, homocystinuria, Huntington's disease, Hutchinson Gilford Progeria Syndrome, hyperoxaluria, primary, hyperphenylalaninemia, hypochondrogenesis, hypochondroplasia, idic15, incontinentia pigmenti, Infantile Gaucher disease, infantile-onset ascending hereditary spastic paralysis, Infertility, Jackson-Weiss syndrome, Joubert syndrome, Juvenile Primary Lateral Sclerosis, Kennedy disease, Klinefelter syndrome, Kniest dysplasia, Krabbe disease, Learning disability, Lesch-Nyhan syndrome, Leukodystrophies, Li-Fraumeni syndrome, lipoprotein lipase deficiency, familial, Male genital disorders, Marfan syndrome, McCune-Albright syndrome, McLeod syndrome, Mediterranean fever, familial, Menkes disease, Menkes syndrome, Metabolic disorders, methemoglobinemia betaglobin type, Methemoglobinemia congenital methaemoglobinaemia, methylmalonic acidemia, Micro syndrome, Microcephaly, Movement disorders, Mowat-Wilson syndrome, Mucopolysaccharidosis (MPS I), Muenke syndrome, Muscular dystrophy, Muscular dystrophy, Duchenne and Becker type, muscular dystrophy, Duchenne and Becker types, myotonic dystrophy, Myotonic dystrophy type 1 and type 2, limb girdle muscular dystrophy, Pompe disease, Neonatal hemochromatosis, neurofibromatosis, neurofibromatosis 1, neurofibromatosis 2, Neurofibromatosis type I, neurofibromatosis type II, Neurologic diseases, Neuromuscular disorders, Niemann-Pick disease, Nonketotic hyperglycinemia, nonsyndromic deafness, Nonsyndromic deafness autosomal recessive, Noonan syndrome, osteogenesis imperfecta (type I and type III), otospondylomegaepiphyseal dysplasia, pantothenate kinase-associated neurodegeneration, Patau Syndrome (Trisomy 13), Pendred syndrome, Peutz-Jeghers syndrome, Pfeiffer syndrome, phenylketonuria, porphyria, porphyria cutanea tarda, Prader-Willi syndrome, primary pulmonary hypertension, prion disease, Progeria, propionic acidemia, protein C deficiency, protein S deficiency, pseudo-Gaucher disease, pseudoxanthoma elasticum, Retinal disorders, retinoblastoma, retinoblastoma FA—Friedreich ataxia, Rett syndrome, Rubinstein-Taybi syndrome, Sandhoff disease, sensory and autonomic neuropathy type III, sickle cell anemia, skeletal muscle regeneration, Skin pigmentation disorders, Smith Lemli Opitz Syndrome, Speech and communication disorders, spinal muscular atrophy, spinal-bulbar muscular atrophy, spinocerebellar ataxia, spondyloepimetaphyseal dysplasia, Strudwick type, spondyloepiphyseal dysplasia congenita, Stickler syndrome, Stickler syndrome COL2A1, Tay-Sachs disease, tetrahydrobiopterin deficiency, thanatophoric dysplasia, thiamine-responsive megaloblastic anemia with diabetes mellitus and sensorineural deafness, Thyroid disease, Tourette's Syndrome, Treacher Collins syndrome, triple X syndrome, tuberous sclerosis, Turner syndrome, Usher syndrome, variegate porphyria, von Hippel-Lindau disease, Waardenburg syndrome, Weissenbacher-Zweymuller syndrome, Wilson disease, Wolf-Hirschhorn syndrome, Xeroderma Pigmentosum, X-linked severe combined immunodeficiency, X-linked sideroblastic anemia, or X-linked spinal-bulbar muscle atrophy.

In some embodiments, the genetic disease is a viral infection. The viral infection may be by a virus selected from the group consisting of an adenovirus, an anellovirus, an arenavirus, an astrovirus, a bunyavirus, a calicivirus, a coronavirus, a filovirus, a flavivirus, a hepadnavirus, a herpesvirus, an orthomyxovirus, a papillomavirus, a paramyxovirus, a parvovirus, a picornavirus, a pneumovirus, a polyomavirus, a poxvirus, a reovirus, a retrovirus, a rhabdovirus, and a togavirus. In some embodiments, the virus is selected from the group consisting of Adeno-associated virus, Aichi virus, Australian bat lyssavirus, BK polyomavirus, Banna virus, Barmah forest virus, Bunyamwera virus, Bunyavirus La Crosse, Bunyavirus snowshoe hare, Cercopithecine herpesvirus, Chandipura virus, Chikungunya virus, Cosavirus A, Cowpox virus, Coxsackievirus, Crimean-Congo hemorrhagic fever virus, Dengue virus, Dhori virus, Dugbe virus, Duvenhage virus, Eastern equine encephalitis virus, Ebolavirus, Echovirus, Encephalomyocarditis virus, Epstein-Barr virus, European bat lyssavirus, GB virus C/Hepatitis G virus, Hantaan virus, Hendra virus, Hepatitis A virus, Hepatitis B virus, Hepatitis C virus, Hepatitis E virus, Hepatitis delta virus, Horsepox virus, Human adenovirus, Human astrovirus, Human coronavirus, Human cytomegalovirus, Human enterovirus 68, Human enterovirus 70, Human herpesvirus 1, Human herpesvirus 2, Human herpesvirus 6, Human herpesvirus 7, Human herpesvirus 8, Human immunodeficiency virus, Human papillomavirus 1, Human papillomavirus 2, Human papillomavirus 16, Human papillomavirus 18, Human parainfluenza, Human parvovirus B19, Human respiratory syncytial virus, Human rhinovirus, Human SARS coronavirus, Human spumaretrovirus, Human T-lymphotropic virus, Human torovirus, Influenza A virus, Influenza B virus, Influenza C virus, Isfahan virus, JC polyomavirus, Japanese encephalitis virus, Junin arenavirus, KI Polyomavirus, Kunjin virus, Lagos bat virus, Lake Victoria Marburg virus, Langat virus, Lassa virus, Lordsdale virus, Louping ill virus, Lymphocytic choriomeningitis virus, Machupo virus, Mayaro virus, MERS coronavirus, Measles virus, Mengo encephalomyocarditis virus, Merkel cell polyomavirus, Mokola virus, Molluscum contagiosum virus, Monkeypox virus, Mumps virus, Murray valley encephalitis virus, New York virus, Nipah virus, Norwalk virus, O'nyong-nyong virus, Orf virus, Oropouche virus, Pichinde virus, Poliovirus, Punta toro phlebovirus, Puumala virus, Rabies virus, Rift valley fever virus, Rosavirus A, Ross river virus, Rotavirus A, Rotavirus B, Rotavirus C, Rubella virus, Sagiyama virus, Salivirus A, Sandfly fever sicilian virus, Sapporo virus, SARS coronavirus 2, Semliki forest virus, Seoul virus, Simian foamy virus, Simian virus 5, Sindbis virus, Southampton virus, St. louis encephalitis virus, Tick-borne powassan virus, Torque teno virus, Toscana virus, Uukuniemi virus, Vaccinia virus, Varicella-zoster virus, Variola virus, Venezuelan equine encephalitis virus, Vesicular stomatitis virus, Western equine encephalitis virus, WU polyomavirus, West Nile virus, Yaba monkey tumor virus, Yaba-like disease virus, Yellow fever virus, and Zika virus.

In some embodiments, the polynucleotide comprises a siRNA, a miRNA, a miRNA mimic, an ASO, or a guide RNA that targets a viral gene. In some embodiments, the polynucleotide comprises a siRNA that targets a viral gene. In some embodiments, the polynucleotide comprises a miRNA that targets a viral gene. In some embodiments, the polynucleotide comprises a miRNA mimic that targets a viral gene. In some embodiments, the polynucleotide comprises an ASO that targets a viral gene. In some embodiments, the polynucleotide comprises a guide RNA that targets a viral gene. The polynucleotide may be conjugated to a targeting molecule that specifically binds to a viral protein or a protein on the surface of a host cell for the virus. In some embodiments, the polynucleotide and the targeting molecule synergize in the treatment of the viral infection.

In some embodiments, the genetic disease is cancer. In some embodiments, the cancer is characterized by overexpression of an oncogene. In some embodiments, the polynucleotide comprises a siRNA, a miRNA, a miRNA mimic, an ASO, or a guide RNA that targets the oncogene. In some embodiments, the polynucleotide comprises a siRNA that targets the oncogene. In some embodiments, the polynucleotide comprises a miRNA that targets the oncogene. In some embodiments, the polynucleotide comprises a miRNA mimic that targets the oncogene. In some embodiments, the polynucleotide comprises an ASO that targets the oncogene. In some embodiments, the polynucleotide comprises a guide RNA that targets the oncogene.

In some embodiments, the cancer is characterized by reduced expression of a tumor suppressor gene. The polynucleotide may comprise a mRNA molecule encoding the tumor suppressor gene. In some embodiments, the polynucleotide comprises a guide RNA that that restores expression of the tumor suppressor gene.

In some embodiments, the genetic disease is a neuromuscular disorder. The neuromuscular disorder may be a muscular dystrophy. In some embodiments, the muscular dystrophy is facioscapulohumeral muscular dystrophy (FSHD). In some embodiments, the polynucleotide comprises a siRNA, a miRNA, a miRNA mimic, an ASO, or a guide RNA that targets DUX4, DMPK or CAPN3. In some embodiments, the polynucleotide comprises a siRNA that targets DUX4. In some embodiments, the polynucleotide comprises a miRNA that targets DUX4. In some embodiments, the polynucleotide comprises a miRNA mimic that targets DUX4. In some embodiments, the polynucleotide comprises an ASO that targets DUX4. In some embodiments, the polynucleotide comprises a guide RNA that targets DUX4. In some embodiments, the ASO that targets DUX is selected from the group consisting of the DUX4-targeted ASOs disclosed in Table 1. In some embodiments, the DUX4-targeted ASO is selected from the group consisting of ASDX2, ASDX4, ASDX23, ASDX26, and ASDX32. In some embodiments, the DUX4-targeted ASO is ASDX2. In some embodiments, the DUX4-targeted ASO is ASDX4. In some embodiments, the DUX4-targeted ASO is ASDX23. In some embodiments, the DUX4-targeted ASO is ASDX26. In some embodiments, the DUX4-targeted ASO is ASDX32. In some embodiments, the polynucleotide comprises a siRNA that targets DMPK. In some embodiments, the polynucleotide comprises a miRNA that targets DMPK. In some embodiments, the polynucleotide comprises a miRNA mimic that targets DMPK. In some embodiments, the polynucleotide comprises an ASO that targets DMPK. In some embodiments, the polynucleotide comprises a siRNA that targets CAPN3. In some embodiments, the polynucleotide comprises a miRNA that targets CAPN3. In some embodiments, the polynucleotide comprises a miRNA mimic that targets CAPN3. In some embodiments, the polynucleotide comprises an ASO that targets CAPN3.

In some embodiments, the muscular dystrophy is Duchenne muscular dystrophy. In some embodiments, the polynucleotide is a mRNA encoding dystrophin or utrophin. In some embodiments, the polynucleotide is a guide RNA that restores the expression of dystrophin or utrophin.

In some embodiments, the polynucleotide is conjugated to a targeting molecule that specifically binds a marker (targeted protein) on the surface of a muscle cell of the subject. The markers are selectively expressed on muscle tissue, are internalized/recycled on a time-scale that allows for drug efficacy (minutes/hours instead of days), and whose expression is not negatively impacted by disease progression. The targeting molecule may specifically bind KLHL41, LMOD2, ENO3, FABP3, CHRNA1, SEMA6C, XIRP2, XIRP1, CAVIN4, CFL2, SVIL, MUSK, ART1, CACNA1S, CDH15, CLCN1, MYMX, or ACTA1. In some embodiments, the targeting molecule specifically binds KLHL41, LMOD2, ENO3, FABP3, CHRNA1, SEMA6C, XIRP2, XIRP1, CAVIN4, CFL2, SVIL, MUSK, ART1, CACNA1S, CDH15, CLCN1, MYMX, or ACTA1; and wherein the polynucleotide is a DUX4-targeted ASO.

In some embodiments, the polynucleotide and the targeting molecule synergize in the treatment of the muscular dystrophy.

In the methods herein, an agent is delivered in a tissue-specific manner, utilizing an agent that specifically binds to a protein expressed on muscle cell surface. An agent that “specifically binds” to a targeted protein, as the term is used herein, is an agent that preferentially or selectively binds to that targeted protein. While certain degree of non-specific interaction may occur between the agent that specifically binds and the targeted protein, nevertheless, specific binding, may be distinguished as mediated through specific recognition of the targeted protein, in whole or part. Typically, specific binding results in a much stronger association between the agent and the targeted protein than between the agent and other proteins, e.g., other muscle proteins. The affinity constant (Ka, as opposed to Kd) of the agent for its cognate is at least 10⁶ or 10⁷, usually at least 10⁸, alternatively at least 10⁹, alternatively at least 10¹⁰, or alternatively at least 10¹¹ M. It should be noted, also, that “specific” binding may be binding that is sufficiently site-specific to effectively be “specific”. For example, when the degree of binding is greater by a higher degree (e.g., equal to or greater than 10-fold, equal to or greater than 20-fold, or even equal to or greater than 100-fold), the binding may become functionally equivalent to binding solely to the targeted protein at a particular location. Directed and effective binding occurs with minimal or no delivery to other tissues. Thus, the amount that is functionally equivalent to specific binding can be determined by assessing whether the goal of effective delivery of agents is met with minimal or no binding to other tissues.

The targeted protein is tissue-specific in certain embodiments, the targeted protein may be present only in one tissue, resulting in tissue-specific interaction between the agent that binds to the targeted protein and the targeted protein itself.

In some embodiments, the targeted protein is enriched in muscle tissue relative to other tissues. “Enriched in muscle tissue relative to other tissues” relates to a surface expression of the targeted protein that is higher than the surface expression on other, non-muscle tissues. In one particular example, the targeted protein is not expressed (i.e., in an amount that is not detectable by the person skilled in the art) on the cell surface of cells comprised in any non-muscle tissue. Non-muscle tissue may be characterized by a non-detectable expression of one or more of Actin alpha 1, myosin heavy chain IV (MYH4), myosin heavy chain VI (MYH6), Myosin IA (Myo1A), or Caveolin 3 (CAV3).

Muscle tissue (or muscular tissue) as used herein relates to soft tissue that makes up the different types of muscles in animals, and give the ability of muscles to contract. Muscle tissue is formed during embryonic development, in a process known as myogenesis. Muscle tissue contains contractile proteins called actin and myosin which contract and relax to cause movement. Among many other muscle proteins present are two regulatory proteins, troponin and tropomyosin. Muscle tissues vary with function and location in the body. In mammals the three types are: skeletal or striated muscle tissue; smooth muscle (non-striated) muscle; and cardiac muscle. In one embodiment muscle tissue is skeletal muscle tissue. Skeletal muscle tissue consists of elongated muscle cells called muscle fibers and is responsible for movements of the body. Other tissues in skeletal muscle include tendons and perimysium. Smooth and cardiac muscle contract involuntarily, without conscious intervention. These muscle types may be activated both through the interaction of the central nervous system as well as by receiving innervation from peripheral plexus or endocrine (hormonal) activation. Striated or skeletal muscle only contracts voluntarily, upon the influence of the central nervous system.

In some embodiments, the targeted protein has stable expression stable or increased expression in diseased tissue relative to normal tissue. A “stable expression” requires that the targeted protein cell surface expression does not decrease during each stage of disease (or pathology) progression. Normal tissue relates to (muscle) tissue not affected by the disease or pathology. Said normal tissue may originate from the subject to be treated or any other subject, which is not affected by the disease or pathology.

In some embodiments, a targeted protein is KLHL41. In some embodiments, a targeted protein is LMOD2. In some embodiments, a targeted protein is ENO3. In some embodiments, a targeted protein is FABP3. In some embodiments, a targeted protein is CHRNA1. In some embodiments, a targeted protein is SEMA6C. In some embodiments, a targeted protein is XIRP2. In some embodiments, a targeted protein is XIRP1. In some embodiments, a targeted protein is CAVIN4. In some embodiments, a targeted protein is CFL2. In some embodiments, a targeted protein is SVIL. In some embodiments, a targeted protein is MUSK. In some embodiments, a targeted protein is ART1. In some embodiments, a targeted protein is CACNA1S. In some embodiments, a targeted protein is CDH15. In some embodiments, a targeted protein is CLCN1. In some embodiments, a targeted protein is CLCN1. In some embodiments, a targeted protein is MYMX. In some embodiments, a targeted protein is ACTA1.

In some embodiments, the methods herein comprise the steps of detecting, comparing, assessing, or any combination thereof. In some cases, the detecting can comprise executing a computer program on a computer. In some cases, the comparing can comprise executing a computer program on a computer. In some cases, the assessing can comprise executing a computer program on a computer. In some embodiments, the detecting, the comparing, the assessing, or any combination thereof employs a computer processor. In some embodiments, the detecting, the comparing, the assessing, or any combination thereof employs computer readable memory. In some embodiments, the detecting, the comparing, the assessing, or any combination thereof employs computer readable instructions on a computer readable memory. In some cases, a computer program can be the same computer program. In some cases, a computer program can be a different computer program. In some cases, a computer herein can comprise a graphical user interface. In some cases, a computer herein can comprise an electronic display.

In some cases, a computer system for implementing the methods herein includes a central processing unit (CPU, also “processor” and “computer processor” herein), which can be a single core or multi core processor, or a plurality of processors for parallel processing. The computer system can also include memory or memory location (e.g., random-access memory, read-only memory, flash memory), electronic storage unit (e.g., hard disk), communication interface (e.g., network adapter) for communicating with one or more other systems, and peripheral devices, such as cache, other memory, data storage and/or electronic display adapters. The memory, storage unit, interface and peripheral devices can be in communication with the CPU through a communication bus, such as a motherboard. The storage unit can be a data storage unit (or data repository) for storing data. The computer system can be operatively coupled to a computer network (“network”) with the aid of the communication interface. The network can be the Internet, an internet and/or extranet, or an intranet and/or extranet that is in communication with the Internet. The network, in some cases is a telecommunication and/or data network. The network can include one or more computer servers, which can enable distributed computing, such as cloud computing. The network, in some cases with the aid of the computer system, can implement a peer-to-peer network, which may enable devices coupled to the computer system to behave as a client or a server. The CPU can execute a sequence of machine-readable instructions, which can be embodied in a program or software. The instructions may be stored in a memory location, such as the memory. The instructions can be directed to the CPU, which can subsequently program or otherwise configure the CPU to implement methods of the present disclosure.

### NUMBERED EMBODIMENTS

A number of methods are disclosed herein. Specific exemplary embodiments of these methods are disclosed below. The following embodiments recite non-limiting permutations of combinations of features disclosed herein. Other permutations of combinations of features are also contemplated. In particular, each of these numbered embodiments is contemplated as depending from or relating to every previous or subsequent numbered embodiment, independent of their order as listed.

### Embodiments Section 1

Embodiment 1. A method of delivering an agent to muscle tissue in vivo in a tissue-specific manner, comprising contacting the surface of muscle cells with an agent that specifically binds a targeted protein expressed on the cell surface of the muscle tissue; wherein the targeted protein is enriched in muscle tissue relative to other tissues; wherein the targeted protein has stable or increased expression in diseased tissue relative to normal tissue; and wherein the targeted protein is internalized and recycled within from about 2 minutes to about 12 hours, from about 2 minutes to about 10 hours, from about 2 minutes to about 8 hours, from about 2 minutes to about 6 hours, from about 2 minutes to about 4 hours, from about 2 minutes to about 2 hours, from about 2 minutes to about 60 minutes, from about 2 minutes to about 40 minutes, from about 2 minutes to about 20 minutes, from about 2 minutes to about 10 minutes, from about 10 minutes to about 12 hours, from about 20 minutes to about 12 hours, from about 40 minutes to about 12 hours, from about 60 minutes to about 12 hours, from about 2 hours to about 12 hours, from about 4 hours to about 12 hours, from about 6 hours to about 12 hours, from about 8 hours to about 12 hours, from about 10 hours to about 12 hours, or from about 11 hours to about 12 hours.

Embodiment 2. The method of embodiment 1, wherein the targeted protein is selected from the group consisting of: KLHL41, LMOD2, ENO3, FABP3, CHRNA1, SEMA6C, XIRP2, XIRP1, CAVIN4, CFL2, SVIL, MUSK, ART1, CACNA1S, CDH15, CLCN1, MYMX, and ACTA1.

Embodiment 3. The method of any one of embodiments 1 to 2, wherein the agent is a specific binding agent of the targeted protein.

Embodiment 4. The method according to embodiment 3, wherein the specific binding agent is a soluble receptor or a soluble ligand.

Embodiment 5. The method according to embodiment 4, wherein the soluble receptor comprises the extracellular domain of a receptor.

Embodiment 6. The method according any one of embodiments 4 or 5, wherein the soluble receptor is a Fc fusion protein.

Embodiment 7. The method of any one of embodiments 1 to 6, wherein the agent is an antibody or an antigen-binding fragment thereof.

Embodiment 8. The method of embodiment 7, wherein the antibody or antigen-binding fragment thereof is selected from the group consisting of a monoclonal antibody, a bispecific antibody, a Fab, a Fab-Fc, a Fv, a single chain Fv (scFv), a diabody, a minibody, a VNAR, and a nanobody.

Embodiment 9. A method of treating a pathology in an individual, comprising administering to the individual a therapeutic targeting agent that specifically binds a targeted protein expressed on muscle tissue cell surface; wherein the targeted protein is enriched in muscle tissue relative to other tissues; wherein the targeted protein has stable or increased expression in diseased tissue relative to normal tissue; and wherein the targeted protein is internalized and recycled within from about 2 minutes to about 12 hours, from about 2 minutes to about 10 hours, from about 2 minutes to about 8 hours, from about 2 minutes to about 6 hours, from about 2 minutes to about 4 hours, from about 2 minutes to about 2 hours, from about 2 minutes to about 60 minutes, from about 2 minutes to about 40 minutes, from about 2 minutes to about 20 minutes, from about 2 minutes to about 10 minutes, from about 10 minutes to about 12 hours, from about 20 minutes to about 12 hours, from about 40 minutes to about 12 hours, from about 60 minutes to about 12 hours, from about 2 hours to about 12 hours, from about 4 hours to about 12 hours, from about 6 hours to about 12 hours, from about 8 hours to about 12 hours, from about 10 hours to about 12 hours, or from about 11 hours to about 12 hours.

Embodiment 10. The method of embodiment 9, wherein the therapeutic targeting agent is an agent that comprises an active agent component and a targeting agent component, wherein the active agent component is selected from the group consisting of: a radionuclide; a chemotherapeutic agent; an immune stimulatory agent; an anti-neoplastic agent; an anti-inflammatory agent; a pro-inflammatory agent; a pro-apoptotic agent; a pro-coagulant; a toxin; an antibiotic; a hormone; an enzyme; a protein; a carrier protein; a lytic agent; a small molecule; aptamers; cells, vaccine-induced or other immune cells; nanoparticles; transferrins; immunoglobulins; multivalent antibodies; lipids; lipoproteins; liposomes; an altered natural ligand; a gene or nucleic acid; an oligonucleotide; RNA; siRNA; an ncRNA mimic; a short-harpin RNA (shRNA); a dicer-dependent siRNA (di-siRNA); an antisense oligonucleotide (ASO); a gapmer; a mixmer; a double-stranded RNA (dsRNA); a single stranded RNAi (ssRNAi); a DNA-directed RNA interference (ddRNAi); an RNA activating oligonucleotide (RNAa); an aptamer; an exon skipping oligonucleotide; a miRNA; a miRNA mimic; an mRNA; a guide RNA; a viral or non-viral gene delivery vector; a prodrug; and a promolecule, and wherein the targeting agent component specifically binds to the targeted protein.

Embodiment 11. The method of embodiment 10, wherein the targeting agent component comprises a specific binding agent of the targeted protein.

Embodiment 12. The method according to embodiment 11, wherein the specific binding agent is a soluble receptor or a soluble ligand.

Embodiment 13. The method according to embodiment 12, wherein the soluble receptor comprises the extracellular domain of a receptor.

Embodiment 14. The method according any one of embodiments 12 or 13, wherein the soluble receptor is a Fc fusion protein.

Embodiment 15. The method of any one of embodiments 10 to 11, wherein the targeting agent component comprises an antibody or an antigen-binding fragment thereof.

Embodiment 16. The method of embodiment 15, wherein the antibody or antigen-binding fragment thereof is selected from the group consisting of a monoclonal antibody, a bispecific antibody, a Fab, a Fab-Fc, a Fv, a single chain Fv (scFv), a diabody, a minibody, a VNAR, and a nanobody.

Embodiment 17. The method of embodiment 10, wherein the active agent component is an oligonucleotide, wherein the targeting agent component is an antibody or an antigen-binding fragment thereof, wherein the active agent component is conjugated to the targeting agent component, and wherein the oligonucleotide targets a disease gene expressed in muscle tissue.

Embodiment 18. The method of any one of embodiments 9 to 17, wherein the targeted protein is selected from the group consisting of: KLHL41, LMOD2, ENO3, FABP3, CHRNA1, SEMA6C, XIRP2, XIRP1, CAVIN4, CFL2, SVIL, MUSK, ART1, CACNA1S, CDH15, CLCN1, MYMX, and ACTA1.

Embodiment 19. A method of delivering an imaging agent to muscle tissue in a tissue-specific manner, comprising contacting the surface of muscle cells with an imaging agent that comprises an imaging agent component and a targeting agent component, wherein the targeting agent component specifically binds to a targeted protein expressed on the cell surface of the tissue; wherein the targeted protein is enriched in muscle tissue relative to other tissues; wherein the targeted protein has stable or increased expression in diseased tissue relative to normal tissue; and wherein the targeted protein is internalized and recycled within from about 2 minutes to about 12 hours, from about 2 minutes to about 10 hours, from about 2 minutes to about 8 hours, from about 2 minutes to about 6 hours, from about 2 minutes to about 4 hours, from about 2 minutes to about 2 hours, from about 2 minutes to about 60 minutes, from about 2 minutes to about 40 minutes, from about 2 minutes to about 20 minutes, from about 2 minutes to about 10 minutes, from about 10 minutes to about 12 hours, from about 20 minutes to about 12 hours, from about 40 minutes to about 12 hours, from about 60 minutes to about 12 hours, from about 2 hours to about 12 hours, from about 4 hours to about 12 hours, from about 6 hours to about 12 hours, from about 8 hours to about 12 hours, from about 10 hours to about 12 hours, or from about 11 hours to about 12 hours.

Embodiment 20. The method of embodiment 19, wherein the targeted protein is selected from the group consisting of: KLHL41, LMOD2, ENO3, FABP3, CHRNA1, SEMA6C, XIRP2, XIRP1, CAVIN4, CFL2, SVIL, MUSK, ART1, CACNA1S, CDH15, CLCN1, MYMX, and ACTA1.

Embodiment 21. The method of any one of embodiments 19 to 20, wherein the targeting agent component is a specific binding agent of the targeted protein.

Embodiment 22. The method according to embodiment 21, wherein the specific binding agent is a soluble receptor or a soluble ligand.

Embodiment 23. The method according to embodiment 22, wherein the soluble receptor comprises the extracellular domain of a receptor.

Embodiment 24. The method according any one of embodiments 22 or 23, wherein the soluble receptor is a Fc fusion protein.

Embodiment 25. The method of any one of embodiments 19 to 21, wherein the targeting agent component is an antibody or an antigen-binding fragment thereof.

Embodiment 26. The method of embodiment 25, wherein the antibody or antigen-binding fragment thereof is selected from the group consisting of a monoclonal antibody, a bispecific antibody, a Fab, a Fab-Fc, a Fv, a single chain Fv (scFv), a diabody, a minibody, a VNAR, and a nanobody.

Embodiment 27. The method of any one of embodiments 19 to 26, wherein the imaging agent component is selected from the group consisting of: a radioactive agent, radioisotope or radiopharmaceutical; a contrast agent; a magnetic agent or a paramagnetic agent; liposomes; ultrasound agents; a gene vector or virus inducing a detecting agent; an enzyme; a prosthetic group; a fluorescent material; a luminescent material; and a bioluminescent material.

Embodiment 28. A method of delivering an imaging agent in a tissue-specific manner to a tissue sample, comprising contacting the tissue sample with an imaging agent that comprises an imaging agent component and a targeting agent component, wherein the targeting agent component specifically binds to a targeted protein expressed on a muscle cell surface of the tissue; wherein the targeted protein is enriched in muscle tissue relative to other tissues; wherein the targeted protein has stable or increased expression in diseased tissue relative to normal tissue; and wherein the targeted protein is internalized and recycled within from about 2 minutes to about 12 hours, from about 2 minutes to about 10 hours, from about 2 minutes to about 8 hours, from about 2 minutes to about 6 hours, from about 2 minutes to about 4 hours, from about 2 minutes to about 2 hours, from about 2 minutes to about 60 minutes, from about 2 minutes to about 40 minutes, from about 2 minutes to about 20 minutes, from about 2 minutes to about 10 minutes, from about 10 minutes to about 12 hours, from about 20 minutes to about 12 hours, from about 40 minutes to about 12 hours, from about 60 minutes to about 12 hours, from about 2 hours to about 12 hours, from about 4 hours to about 12 hours, from about 6 hours to about 12 hours, from about 8 hours to about 12 hours, from about 10 hours to about 12 hours, or from about 11 hours to about 12 hours.

Embodiment 29. The method of embodiment 28, wherein the targeted protein is selected from the group consisting of: KLHL41, LMOD2, ENO3, FABP3, CHRNA1, SEMA6C, XIRP2, XIRP1, CAVIN4, CFL2, SVIL, MUSK, ART1, CACNA1S, CDH15, CLCN1, MYMX, and ACTA1.

Embodiment 30. The method of any one of embodiments 28 to 29, wherein the targeting agent component is a specific binding agent of the targeted protein.

Embodiment 31. The method according to embodiment 30, wherein the specific binding agent is a soluble receptor or a soluble ligand.

Embodiment 32. The method according to embodiment 31, wherein the soluble receptor comprises the extracellular domain of a receptor.

Embodiment 33. The method according any one of embodiments 31 or 32, wherein the soluble receptor is a Fc fusion protein.

Embodiment 34. The method of any one of embodiments 28 to 30, wherein the targeting agent component is an antibody or an antigen-binding fragment thereof.

Embodiment 35. The method of embodiment 34, wherein the antibody or antigen-binding fragment thereof is selected from the group consisting of a monoclonal antibody, a bispecific antibody, a Fab, a Fab-Fc, a Fv, a single chain Fv (scFv), a diabody, a minibody, a VNAR, and a nanobody.

Embodiment 36. The method of any one of embodiments 28 to 35, wherein the imaging agent component is selected from the group consisting of: a radioactive agent, radioisotope or radiopharmaceutical; a contrast agent; a magnetic agent or a paramagnetic agent; liposomes; ultrasound agents; a gene vector or virus inducing a detecting agent; an enzyme; a prosthetic group; a fluorescent material; a luminescent material; and a bioluminescent material.

Embodiment 37. A method of performing physical imaging of muscle tissue of an individual, comprising administering to the individual an imaging agent comprising a targeting agent component and an imaging agent component, wherein the targeting agent component specifically binds to a targeted protein expressed on the cell surface of the muscle tissue; wherein the targeted protein is enriched in muscle tissue relative to other tissues; wherein the targeted protein has stable or increased expression in diseased tissue relative to normal tissue; and wherein the targeted protein is internalized and recycled within from about 2 minutes to about 12 hours, from about 2 minutes to about 10 hours, from about 2 minutes to about 8 hours, from about 2 minutes to about 6 hours, from about 2 minutes to about 4 hours, from about 2 minutes to about 2 hours, from about 2 minutes to about 60 minutes, from about 2 minutes to about 40 minutes, from about 2 minutes to about 20 minutes, from about 2 minutes to about 10 minutes, from about 10 minutes to about 12 hours, from about 20 minutes to about 12 hours, from about 40 minutes to about 12 hours, from about 60 minutes to about 12 hours, from about 2 hours to about 12 hours, from about 4 hours to about 12 hours, from about 6 hours to about 12 hours, from about 8 hours to about 12 hours, from about 10 hours to about 12 hours, or from about 11 hours to about 12 hours.

Embodiment 38. The method of embodiment 37, wherein the targeted protein is selected from the group consisting of: KLHL41, LMOD2, ENO3, FABP3, CHRNA1, SEMA6C, XIRP2, XIRP1, CAVIN4, CFL2, SVIL, MUSK, ART1, CACNA1S, CDH15, CLCN1, MYMX, and ACTA1.

Embodiment 39. The method of any one of embodiments 37 to 38, wherein the imaging agent component is selected from the group consisting of: a radioactive agent, radioisotope or radiopharmaceutical; a contrast agent; a magnetic agent or a paramagnetic agent; liposomes; ultrasound agents; a gene vector or virus inducing a detecting agent; an enzyme; a prosthetic group; a fluorescent material; a luminescent material; and a bioluminescent material.

Embodiment 40. A method of assessing an individual for the presence or absence of a muscle tissue pathology, comprising: a) administering to the individual an imaging agent that comprises an imaging agent component and a targeting agent component, wherein the targeting agent component specifically binds to a targeted protein expressed on the cell surface of the muscle tissue, and b) assessing the individual for the presence or absence of a concentration of the imaging agent, c) wherein the presence or absence of a concentration of the imaging agent is indicative of the presence of the pathology.

Embodiment 41. The method of embodiment 40, wherein the targeting agent component is a specific binding agent of the targeted protein.

Embodiment 42. The method according to embodiment 41, wherein the specific binding agent is a soluble receptor or a soluble ligand.

Embodiment 43. The method according to embodiment 42, wherein the soluble receptor comprises the extracellular domain of a receptor.

Embodiment 44. The method according any one of embodiments 42 or 43, wherein the soluble receptor is a Fc fusion protein.

Embodiment 45. The method of any one of embodiments 40 to 41, wherein the targeting agent component is an antibody or an antigen-binding fragment thereof.

Embodiment 46. The method of embodiment 45, wherein the antibody or antigen-binding fragment thereof is selected from the group consisting of a monoclonal antibody, a bispecific antibody, a Fab, a Fab-Fc, a Fv, a single chain Fv (scFv), a diabody, a minibody, a VNAR, and a nanobody.

Embodiment 47. The method any one of embodiments 40 to 46, wherein the targeted protein is selected from the group consisting of: KLHL41, LMOD2, ENO3, FABP3, CHRNA1, SEMA6C, XIRP2, XIRP1, CAVIN4, CFL2, SVIL, MUSK, ART1, CACNA1S, CDH15, CLCN1, MYMX, and ACTA1.

Embodiment 48. The method of any one of embodiments 40 to 47, wherein the imaging agent component is selected from the group consisting of: a radioactive agent, radioisotope or radiopharmaceutical; a contrast agent; a magnetic agent or a paramagnetic agent; liposomes; ultrasound agents; a gene vector or virus inducing a detecting agent; an enzyme; a prosthetic group; a fluorescent material; a luminescent material; and a bioluminescent material.

Embodiment 49. A method of assessing response of muscle tissue from an individual to treatment with a therapeutic targeting agent, wherein the therapeutic targeting agent specifically binds a targeted protein expressed on the cell surface of the muscle tissue, comprising: a) assessing the level of the targeted protein in a sample from the individual before treatment with the therapeutic targeting agent; b) assessing the level of the targeted protein in a sample from the individual during or after treatment with the therapeutic targeting agent; c) comparing the level before treatment with the level during or after treatment, wherein a level of the targeted protein during or after treatment that is significantly lower than the level of the targeted protein before treatment, is indicative of efficacy of treatment with the therapeutic targeting agent; wherein the targeted protein is enriched in muscle tissue relative to other tissues; wherein the targeted protein has stable or increased expression in diseased tissue relative to normal tissue; and wherein the targeted protein is internalized and recycled within from about 2 minutes to about 12 hours, from about 2 minutes to about 10 hours, from about 2 minutes to about 8 hours, from about 2 minutes to about 6 hours, from about 2 minutes to about 4 hours, from about 2 minutes to about 2 hours, from about 2 minutes to about 60 minutes, from about 2 minutes to about 40 minutes, from about 2 minutes to about 20 minutes, from about 2 minutes to about 10 minutes, from about 10 minutes to about 12 hours, from about 20 minutes to about 12 hours, from about 40 minutes to about 12 hours, from about 60 minutes to about 12 hours, from about 2 hours to about 12 hours, from about 4 hours to about 12 hours, from about 6 hours to about 12 hours, from about 8 hours to about 12 hours, from about 10 hours to about 12 hours, or from about 11 hours to about 12 hours.

Embodiment 50. The method of embodiment 49, wherein the targeting protein is selected from the group consisting of: KLHL41, LMOD2, ENO3, FABP3, CHRNA1, SEMA6C, XIRP2, XIRP1, CAVIN4, CFL2, SVIL, MUSK, ART1, CACNA1S, CDH15, CLCN1, MYMX, and ACTA1.

### Embodiments Section 2

Embodiment 1. A method of delivering an agent to muscle tissue in vivo in a tissue-specific or tissue selective manner, the method comprising contacting the surface of muscle cell(s) of the muscle tissue with an effective amount of an agent that specifically or selective binds a targeted protein expressed on the cell surface of the muscle cell(s) of the muscle tissue; wherein the targeted protein is enriched in muscle tissue relative to other tissues; wherein the targeted protein has stable or increased expression in diseased tissue relative to otherwise comparable normal tissue; and optionally wherein the targeted protein is internalized and recycled within from about 2 minutes to about 12 hours, from about 2 minutes to about 10 hours, from about 2 minutes to about 8 hours, from about 2 minutes to about 6 hours, from about 2 minutes to about 4 hours, from about 2 minutes to about 2 hours, from about 2 minutes to about 60 minutes, from about 2 minutes to about 40 minutes, from about 2 minutes to about 20 minutes, from about 2 minutes to about 10 minutes, from about 10 minutes to about 12 hours, from about 20 minutes to about 12 hours, from about 40 minutes to about 12 hours, from about 60 minutes to about 12 hours, from about 2 hours to about 12 hours, from about 4 hours to about 12 hours, from about 6 hours to about 12 hours, from about 8 hours to about 12 hours, from about 10 hours to about 12 hours, or from about 11 hours to about 12 hours; thereby delivering an agent to muscle tissue in vivo in a tissue-specific or tissue selective manner.

Embodiment 2. The method of embodiment 1, wherein the targeted protein is expressed from a gene selected from the group consisting of: KLHL41, LMOD2, ENO3, FABP3, CHRNA1, SEMA6C, XIRP2, XIRP1, CAVIN4, CFL2, SVIL, MUSK, ART1, CACNA1S, CDH15, CLCN1, MYMX, and ACTA1.

Embodiment 3. The method of any one of embodiments 1 to 2, wherein the agent is a specific or selective binding agent of the targeted protein.

Embodiment 4. The method according to embodiment 3, wherein the specific or selective binding agent is a soluble receptor or a soluble ligand.

Embodiment 5. The method according to embodiment 4, wherein specific or selective binding agent is the soluble receptor, and wherein the soluble receptor comprises the extracellular domain of a receptor.

Embodiment 6. The method according any one of embodiments 4 or 5, wherein the soluble receptor is a Fc fusion protein.

Embodiment 7. The method of any one of embodiments 1 to 6, wherein the agent is an antibody or an antigen-binding fragment thereof.

Embodiment 8. The method of embodiment 7, wherein the antibody or antigen-binding fragment thereof is selected from the group consisting of a monoclonal antibody, a bispecific antibody, a Fab, a Fab-Fc, a Fv, a single chain Fv (scFv), a diabody, a minibody, a VNAR, and an immunoglobulin single variable domain (ISV).

Embodiment 9. A method of treating a pathology in an individual, comprising administering to the individual a therapeutically effective amount of a therapeutic targeting agent that specifically or selectively binds a targeted protein expressed on muscle tissue cell surface; wherein the targeted protein is enriched in muscle tissue relative to other tissues; wherein the targeted protein has stable or increased expression in diseased tissue relative to otherwise comparable normal tissue; and optionally wherein the targeted protein is internalized and recycled within from about 2 minutes to about 12 hours, from about 2 minutes to about 10 hours, from about 2 minutes to about 8 hours, from about 2 minutes to about 6 hours, from about 2 minutes to about 4 hours, from about 2 minutes to about 2 hours, from about 2 minutes to about 60 minutes, from about 2 minutes to about 40 minutes, from about 2 minutes to about 20 minutes, from about 2 minutes to about 10 minutes, from about 10 minutes to about 12 hours, from about 20 minutes to about 12 hours, from about 40 minutes to about 12 hours, from about 60 minutes to about 12 hours, from about 2 hours to about 12 hours, from about 4 hours to about 12 hours, from about 6 hours to about 12 hours, from about 8 hours to about 12 hours, from about 10 hours to about 12 hours, or from about 11 hours to about 12 hours; thereby treating the pathology in the individual.

Embodiment 10. The method of embodiment 9, wherein the therapeutic targeting agent is an agent that comprises an active agent component and a targeting agent component, wherein the active agent component is selected from the group consisting of: a radionuclide; a chemotherapeutic agent; an immune stimulatory agent; an anti-neoplastic agent; an anti-inflammatory agent; a pro-inflammatory agent; a pro-apoptotic agent; a pro-coagulant; a toxin; an antibiotic; a hormone; an enzyme; a protein; a carrier protein; a lytic agent; a small molecule; aptamers; cells, vaccine-induced or other immune cells; nanoparticles; transferrins; immunoglobulins; multivalent antibodies; lipids; lipoproteins; liposomes; an altered natural ligand; a gene or nucleic acid; an oligonucleotide; RNA; siRNA; an ncRNA mimic; a short-harpin RNA (shRNA); a dicer-dependent siRNA (di-siRNA); an antisense oligonucleotide (ASO); a gapmer; a mixmer; a double-stranded RNA (dsRNA); a single stranded RNAi (ssRNAi); a DNA-directed RNA interference (ddRNAi); an RNA activating oligonucleotide (RNAa); an aptamer; an exon skipping oligonucleotide; a miRNA; a miRNA mimic; an mRNA; a guide RNA; a viral or non-viral gene delivery vector; a prodrug; and a promolecule, and wherein the targeting agent component specifically or selectively binds to the targeted protein.

Embodiment 11. The method of embodiment 10, wherein the targeting agent component comprises a specific or selective binding agent for the targeted protein.

Embodiment 12. The method according to embodiment 11, wherein the specific or selective binding agent is a soluble receptor or a soluble ligand.

Embodiment 13. The method according to embodiment 12, wherein the specific or selective binding agent is a soluble receptor, and wherein the soluble receptor comprises the extracellular domain of a receptor.

Embodiment 14. The method according any one of embodiments 12 or 13, wherein the soluble receptor is a Fc fusion protein or a biologically active fragment thereof.

Embodiment 15. The method of any one of embodiments 10 to 11, wherein the targeting agent component comprises an antibody or an antigen-binding fragment thereof.

Embodiment 16. The method of embodiment 15, wherein the antibody or antigen-binding fragment thereof is selected from the group consisting of a monoclonal antibody, a bispecific antibody, a Fab, a Fab-Fc, a Fv, a single chain Fv (scFv), a diabody, a minibody, a VNAR, and an immunoglobulin single variable domain (ISV).

Embodiment 17. The method of embodiment 10, wherein the active agent component is an oligonucleotide or polynucleotide, wherein the targeting agent component is an antibody or an antigen-binding fragment thereof, wherein the active agent component is conjugated to the targeting agent component, and wherein the oligonucleotide targets a disease gene expressed in muscle tissue.

Embodiment 18. The method of any one of embodiments 9 to 10, wherein the targeted protein is encoded by a gene selected from the group consisting of: KLHL41, LMOD2, ENO3, FABP3, CHRNA1, SEMA6C, XIRP2, XIRP1, CAVIN4, CFL2, SVIL, MUSK, ART1, CACNA1S, CDH15, CLCN1, MYMX, and ACTA1.

Embodiment 19. A method of delivering an imaging agent to muscle tissue in a tissue-specific or tissue-selective manner, comprising contacting the surface of muscle cell(s) of the muscle tissue with an effective amount of an imaging agent that comprises an imaging agent component and a targeting agent component, wherein the targeting agent component specifically or selectively binds to a targeted protein expressed on the cell surface of the muscle cells of the muscle tissue; wherein the targeted protein is enriched in muscle tissue relative to other tissues; wherein the targeted protein has stable or increased expression in diseased tissue relative to otherwise comparable normal tissue; and optionally wherein the targeted protein is internalized and recycled within from about 2 minutes to about 12 hours, from about 2 minutes to about 10 hours, from about 2 minutes to about 8 hours, from about 2 minutes to about 6 hours, from about 2 minutes to about 4 hours, from about 2 minutes to about 2 hours, from about 2 minutes to about 60 minutes, from about 2 minutes to about 40 minutes, from about 2 minutes to about 20 minutes, from about 2 minutes to about 10 minutes, from about 10 minutes to about 12 hours, from about 20 minutes to about 12 hours, from about 40 minutes to about 12 hours, from about 60 minutes to about 12 hours, from about 2 hours to about 12 hours, from about 4 hours to about 12 hours, from about 6 hours to about 12 hours, from about 8 hours to about 12 hours, from about 10 hours to about 12 hours, or from about 11 hours to about 12 hours; thereby delivering an imaging agent to muscle tissue in a tissue-specific or tissue-selective manner

Embodiment 20. The method of embodiment 19, wherein the targeted protein is expressed from a gene selected from the group consisting of: KLHL41, LMOD2, ENO3, FABP3, CHRNA1, SEMA6C, XIRP2, XIRP1, CAVIN4, CFL2, SVIL, MUSK, ART1, CACNA1S, CDH15, CLCN1, MYMX, and ACTA1.

Embodiment 21. The method of any one of embodiments 19 to 20, wherein the targeting agent component is a specific or selective binding agent of the targeted protein.

Embodiment 22. The method according to embodiment 21, wherein the specific or selective binding agent is a soluble receptor or a soluble ligand.

Embodiment 23. The method according to embodiment 22, wherein the specific or selective binding agent is a soluble receptor, and wherein the soluble receptor comprises an extracellular domain of a receptor.

Embodiment 24. The method according any one of embodiments 22 or 23, wherein the soluble receptor is a Fc fusion protein.

Embodiment 25. The method of any one of embodiments 19 to 21, wherein the targeting agent component is an antibody or an antigen-binding fragment thereof.

Embodiment 26. The method of embodiment 25, wherein the antibody or antigen-binding fragment thereof is selected from the group consisting of a monoclonal antibody, a bispecific antibody, a Fab, a Fab-Fc, a Fv, a single chain Fv (scFv), a diabody, a minibody, a VNAR, and an immunoglobulin single variable domain (ISV).

Embodiment 27. The method of any one of embodiments 19 to 26, wherein the imaging agent component is selected from the group consisting of: a radioactive agent, radioisotope or radiopharmaceutical; a contrast agent; a magnetic agent or a paramagnetic agent; liposomes; ultrasound agents; a gene vector or virus inducing a detecting agent; an enzyme; a prosthetic group; a fluorescent material; a luminescent material; and a bioluminescent material.

Embodiment 28. A method of delivering an imaging agent in a tissue-specific or selective manner to a tissue sample, the method comprising contacting the tissue sample with an effective amount of an imaging agent that comprises an imaging agent component and a targeting agent component, wherein the targeting agent component specifically or selectively binds to a targeted protein expressed on a muscle cell surface of the tissue; wherein the targeted protein is enriched in muscle tissue relative to other tissues; wherein the targeted protein has stable or increased expression in diseased tissue relative to otherwise comparable normal tissue; and optionally wherein the targeted protein is internalized and recycled within from about 2 minutes to about 12 hours, from about 2 minutes to about 10 hours, from about 2 minutes to about 8 hours, from about 2 minutes to about 6 hours, from about 2 minutes to about 4 hours, from about 2 minutes to about 2 hours, from about 2 minutes to about 60 minutes, from about 2 minutes to about 40 minutes, from about 2 minutes to about 20 minutes, from about 2 minutes to about 10 minutes, from about 10 minutes to about 12 hours, from about 20 minutes to about 12 hours, from about 40 minutes to about 12 hours, from about 60 minutes to about 12 hours, from about 2 hours to about 12 hours, from about 4 hours to about 12 hours, from about 6 hours to about 12 hours, from about 8 hours to about 12 hours, from about 10 hours to about 12 hours, or from about 11 hours to about 12 hours; thereby delivering an imaging agent in a tissue-specific or selective manner to a tissue sample.

Embodiment 29. The method of embodiment 28, wherein the targeted protein is expressed from a gene selected from the group consisting of: KLHL41, LMOD2, ENO3, FABP3, CHRNA1, SEMA6C, XIRP2, XIRP1, CAVIN4, CFL2, SVIL, MUSK, ART1, CACNA1S, CDH15, CLCN1, MYMX, and ACTA1.

Embodiment 30. The method of any one of embodiments 28 to 29, wherein the targeting agent component is a specific or selective binding agent of the targeted protein.

Embodiment 31. The method according to embodiment 30, wherein the specific or selective binding agent is a soluble receptor or a soluble ligand.

Embodiment 32. The method according to embodiment 31, wherein the specific or selective binding agent is the soluble receptor, and wherein the soluble receptor comprises the extracellular domain of a receptor.

Embodiment 33. The method according any one of embodiments 31 or 32, wherein the soluble receptor is a Fc fusion protein.

Embodiment 34. The method of any one of embodiments 28 to 30, wherein the targeting agent component is an antibody or an antigen-binding fragment thereof.

Embodiment 35. The method of embodiment 34, wherein the antibody or antigen-binding fragment thereof is selected from the group consisting of a monoclonal antibody, a bispecific antibody, a Fab, a Fab-Fc, a Fv, a single chain Fv (scFv), a diabody, a minibody, a VNAR, and an immunoglobulin single variable domain (ISV).

Embodiment 36. The method of any one of embodiments 28 to 35, wherein the imaging agent component is selected from the group consisting of: a radioactive agent, radioisotope or radiopharmaceutical; a contrast agent; a magnetic agent or a paramagnetic agent; liposomes; ultrasound agents; a gene vector or virus inducing a detecting agent; an enzyme; a prosthetic group; a fluorescent material; a luminescent material; and a bioluminescent material.

Embodiment 37. A method of performing physical imaging of muscle tissue of an individual, the method comprising administering to the individual an effective amount of an imaging agent comprising a targeting agent component and an imaging agent component, wherein the targeting agent component specifically or selectively binds to a targeted protein expressed on the cell surface of the muscle tissue; wherein the targeted protein is enriched in muscle tissue relative to other tissues; wherein the targeted protein has stable or increased expression in diseased tissue relative to otherwise comparable normal tissue; and optionally wherein the targeted protein is internalized and recycled within from about 2 minutes to about 12 hours, from about 2 minutes to about 10 hours, from about 2 minutes to about 8 hours, from about 2 minutes to about 6 hours, from about 2 minutes to about 4 hours, from about 2 minutes to about 2 hours, from about 2 minutes to about 60 minutes, from about 2 minutes to about 40 minutes, from about 2 minutes to about 20 minutes, from about 2 minutes to about 10 minutes, from about 10 minutes to about 12 hours, from about 20 minutes to about 12 hours, from about 40 minutes to about 12 hours, from about 60 minutes to about 12 hours, from about 2 hours to about 12 hours, from about 4 hours to about 12 hours, from about 6 hours to about 12 hours, from about 8 hours to about 12 hours, from about 10 hours to about 12 hours, or from about 11 hours to about 12 hours; thereby performing physical imaging of muscle tissue of an individual.

Embodiment 38. The method of embodiment 37, wherein the targeted protein is selected expressed from a gene selected from the group consisting of: KLHL41, LMOD2, ENO3, FABP3, CHRNA1, SEMA6C, XIRP2, XIRP1, CAVIN4, CFL2, SVIL, MUSK, ART1, CACNA1S, CDH15, CLCN1, MYMX, and ACTA1.

Embodiment 39. The method of any one of embodiments 37 to 38, wherein the imaging agent component is selected from the group consisting of: a radioactive agent, radioisotope or radiopharmaceutical; a contrast agent; a magnetic agent or a paramagnetic agent; liposomes; ultrasound agents; a gene vector or virus inducing a detecting agent; an enzyme; a prosthetic group; a fluorescent material; a luminescent material; and a bioluminescent material.

Embodiment 40. A method of assessing an individual for the presence or absence of a muscle tissue pathology, the method comprising: a) administering to the individual an effective amount of an imaging agent that comprises an imaging agent component and a targeting agent component, wherein the targeting agent component specifically or selectively binds to a targeted protein expressed on the cell surface of the muscle tissue, and b) detecting by using a MRI or other medical imaging, an in vitro diagnostic, or any combination thereof in the individual for the presence or absence of a concentration of the imaging agent, c) wherein the presence or absence of a concentration of the imaging agent is indicative of the presence of the pathology; thereby assessing an individual for the presence or absence of a muscle tissue pathology.

Embodiment 41. The method of embodiment 40, wherein the targeting agent component is a specific or selective binding agent of the targeted protein.

Embodiment 42. The method according to embodiment 41, wherein the specific or selective binding agent is a soluble receptor or a soluble ligand.

Embodiment 43. The method according to embodiment 42, wherein the specific or the selective binding agent is the soluble receptor that comprises the extracellular domain of a receptor.

Embodiment 44. The method according any one of embodiments 42 or 43, wherein the soluble receptor is a Fc fusion protein.

Embodiment 45. The method of any one of embodiments 40 to 41, wherein the targeting agent component is an antibody or an antigen-binding fragment thereof.

Embodiment 46. The method of embodiment 45, wherein the antibody or antigen-binding fragment thereof is selected from the group consisting of a monoclonal antibody, a bispecific antibody, a Fab, a Fab-Fc, a Fv, a single chain Fv (scFv), a diabody, a minibody, a VNAR, and an immunoglobulin single variable domain (ISV).

Embodiment 47. The method any one of embodiments 40 to 46, wherein the targeted protein is expressed from a gene selected from the group consisting of: KLHL41, LMOD2, ENO3, FABP3, CHRNA1, SEMA6C, XIRP2, XIRP1, CAVIN4, CFL2, SVIL, MUSK, ART1, CACNA1S, CDH15, CLCN1, MYMX, and ACTA1.

Embodiment 48. The method of any one of embodiments 40 to 47, wherein the imaging agent component is selected from the group consisting of: a radioactive agent, radioisotope or radiopharmaceutical; a contrast agent; a magnetic agent or a paramagnetic agent; liposomes; ultrasound agents; a gene vector or virus inducing a detecting agent; an enzyme; a prosthetic group; a fluorescent material; a luminescent material; and a bioluminescent material.

Embodiment 49. A method of assessing response of muscle tissue from an individual to treatment with a therapeutic targeting agent, wherein the therapeutic targeting agent specifically binds a targeted protein expressed on the cell surface of the muscle tissue, the method comprising: a) determining a level of the targeted protein in a sample obtained from the individual before treatment with the therapeutic targeting agent; b) detecting by using an MRI or other medical imaging, an in vitro diagnostic, or any combination thereof, the level of the targeted protein in a sample obtained from the individual during or after treatment with the therapeutic targeting agent; c) optionally, comparing using a computer the level before treatment with the level during or after treatment, wherein a level of the targeted protein during or after treatment that is lower than the level of the targeted protein before treatment, is indicative of efficacy of treatment with the therapeutic targeting agent; wherein the targeted protein is enriched in muscle tissue relative to other tissues; wherein the targeted protein has stable or increased expression in diseased tissue relative to otherwise comparable normal tissue; and optionally wherein the targeted protein is internalized and recycled within from about 2 minutes to about 12 hours, from about 2 minutes to about 10 hours, from about 2 minutes to about 8 hours, from about 2 minutes to about 6 hours, from about 2 minutes to about 4 hours, from about 2 minutes to about 2 hours, from about 2 minutes to about 60 minutes, from about 2 minutes to about 40 minutes, from about 2 minutes to about 20 minutes, from about 2 minutes to about 10 minutes, from about 10 minutes to about 12 hours, from about 20 minutes to about 12 hours, from about 40 minutes to about 12 hours, from about 60 minutes to about 12 hours, from about 2 hours to about 12 hours, from about 4 hours to about 12 hours, from about 6 hours to about 12 hours, from about 8 hours to about 12 hours, from about 10 hours to about 12 hours, or from about 11 hours to about 12 hours; thereby assessing response of muscle tissue from an individual to treatment with a therapeutic targeting agent.

Embodiment 50. The method of embodiment 49, wherein the targeting protein is expressed from a gene selected from the group consisting of: KLHL41, LMOD2, ENO3, FABP3, CHRNA1, SEMA6C, XIRP2, XIRP1, CAVIN4, CFL2, SVIL, MUSK, ART1, CACNA1S, CDH15, CLCN1, MYMX, and ACTA1.

Embodiment 51. The method of embodiment 9, wherein the pathology comprises a neuromuscular disorder including a muscular dystrophy or a myopathy.

Embodiment 52. The method of embodiment 51, wherein the pathology is a Duchenne's muscular dystrophy (DMD), Myotonic Dystrophy (MD), Facioscapulohumeral muscular dystrophy (FSHD), Limb-Girdle muscular dystrophy (LGMD), Becker muscular dystrophy, Oculopharyngeal muscular dystrophy, Emery-Dreifuss muscular dystrophy, or Distal muscular dystrophy.

Embodiment 53. The method of any preceding embodiment, wherein the individual is a human.

Embodiment 54. The method of any preceding embodiment, administering comprises oral, by inhalation, intranasal, by injection, subcutaneous, intramuscular, administering directly to a tissue or call, administering indirectly to a tissue or a cell, intravenously, rectally, intrathecal, intra ocular, in the ear, intraperitoneal, or topical.

Embodiment 55. The method of any preceding embodiment, wherein the contacting occurs in an individual, and the contacting results from administering an agent to the individual.

Embodiment 56. The method of any preceding embodiment, wherein the assessing comprises a biopsy.

Embodiment 57. The method of any preceding embodiment, wherein the agent is administered in the form of a pharmaceutical composition that further comprises a pharmaceutically acceptable: excipient, diluent, carrier, or any combination thereof.

Embodiment 58. The method of any preceding embodiment, wherein the contacting or the administering is: once per day, twice per day, three times per day, four times per day, weekly, twice a week, three times a week, four times a week, monthly, bi-monthly, every third month, quarterly, twice a year, yearly, as needed, or for life.

Embodiment 59. The method of embodiment 57, wherein the pharmaceutical composition is in unit dose form.

Embodiment 60. The method of any preceding embodiment, wherein the agent is administered in an amount ranging from about 1 ng to about 25,000 mg, about 10 ng, about 100 ng, about 1 microgram, about 10 micrograms, about 100 micrograms, about 1 mg, about 10 mg, about 100 mg, about 1000 mg, about 10000 mg, or about 25000 mg.

Embodiment 61. The method of any preceding embodiment, wherein the agent is in the form of salt that is optionally pharmaceutically acceptable.

### EXAMPLES

### Example 1: Identification of Targets for Muscle-Specific Delivery

To identify ideal targets to enable muscle specific delivery of ADCs, a meta-analysis of transcriptomic gene expression from human disease and healthy muscle tissue from 28 datasets from 21 different muscle related disorders was performed. These are listed in Table 2, infra.

TABLE 2
Datasets of muscle related disorders

| Disease | Publication Dataset |
|---|---|
| DMD | PMID_11121445 |
| DMD | PMID_12415109 |
| DMD | GSE38417 |
| FSHD | PMID_14519683 |
| FSHD | PMID: 22988124 |
| FSHD | PMID: 24861551 |
| Dysferlinopathy | PMID: 12471055 |
| juvenilie_dermatomyositis | PMID_11937576 |
| Dermatomyositis | PMID: 23171592 |
| Caveolinopathy | PMID: 23171592 |
| Nemaline_myopathy | PMID_12677001 |
| myotonic_dystrophy_type_1_{—} | PMID: 20066428 |
| myotonic_dystrophy_type_1_{—} | PMID: 22758909 |
| myotonic_dystrophy_type_2 | PMID: 20971734 |
| Spastic paraplegia | PMID_15079007 |
| Strabismic | PMID: 22786898 |
| Ischemic cardiomyopathy | PMID: 20332183 |
| familiar cardiomyopathy | GSE1145 |
| Hypertrophic cardiomyopathy | GSE1145 |
| Idiopathic cardiomyopathy | GSE1145 |
| Post partum cardiomyopathy | GSE1145 |
| Dilated cardiomyopathy | PMID: 20460602 |
| Familial type_2 diabetes_{—} | PMID: 21393865 |
| Non-familial type_2 diabetes | PMID: 27689005 |
| type_2 diabetes DM2 or IGT | PMID: 21803291 |
| Hibernating myocardium | AE_SERIES_E-MIMR-27 |
| Parkinsonism and ataxia | GSE18715 |
| Pompe disease | PMID: 22658377 |

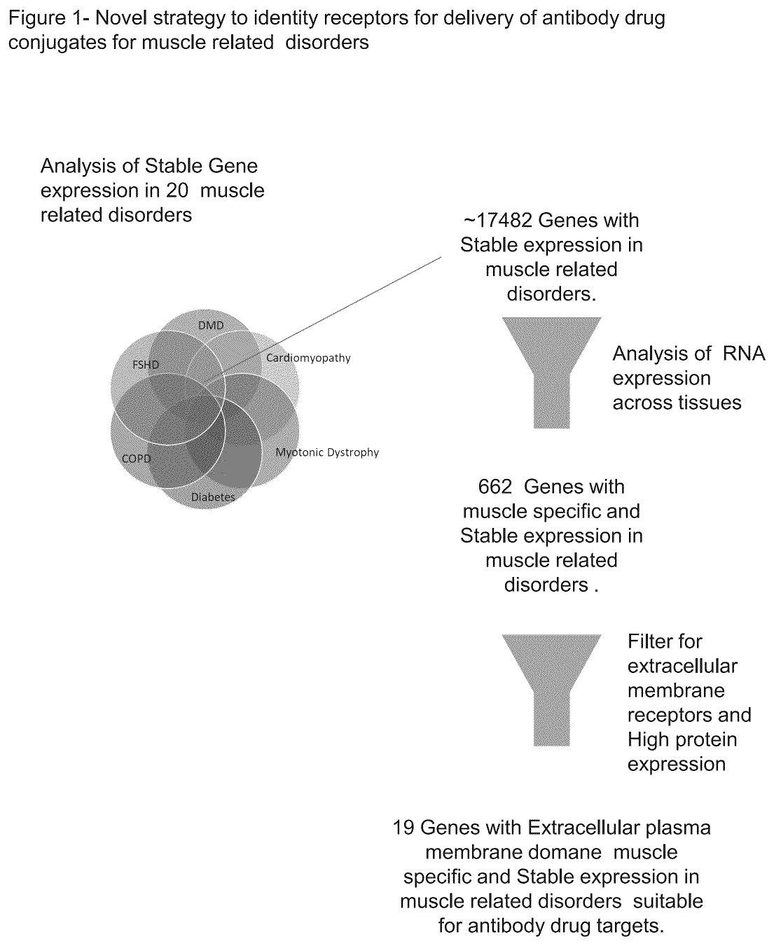

An overview of this analysis is displayed in FIG. 1. Initially genes with stable or increased expression in diseased muscle tissue compared to control muscle tissue were identified, using the dataset's and statistical methods from each study. This data was overlapped, identifying ˜17,000 genes that remain stably expressed across all muscle disorders analyzed. Next, RNA expression data from the GTEx (https://gtexportal.org/home/) and FANTOM5 (PMID: 24670764) databases was utilized to identify genes that are significantly enriched in muscle tissue (skeletal, smooth, cardiac and tongue muscle) versus non muscle tissue. Filtering the list to only stable expressed disease genes resulted in the identification of ˜600 genes. To identify genes that would be suitable for antibody targeting, the gene ontology database (PMID: 10802651) was used to identify genes that were listed as extracellular and associated with the outer plasma membrane, resulting in a list of about ˜120 genes.

Finally, the RNA expression data was validated at the protein expression level using the human protein atlas (PMID: 25613900) so that protein and RNA expression data both showed extracellular expression and muscle specificity. This data was filtered for genes that were highly-expressed at the protein level, i.e., genes that were expressed at expression level 2 or 3 (moderate to high, if available) according to the immunohistochemistry scoring data available in the human protein atlas. This resulted in a list of 19 genes displayed in Table 3, infra.

TABLE 3
Ideal muscle specific ADC targets for muscle related Disorders:

KLHL41
LMOD2
ENO3
FABP3
CHRNA1
SEMA6C
XIRP2
XIRP1
CAVIN4
CFL2
SVIL
MUSK
ART1
CACNA1S
CDH15
CLCN1
MYMX
ACTA1
SLC2A4 (GLUT4)

As validation of the soundness of this approach, it is noted that previous studies have suggested that SLC2A4 (GLUT4), would be a good target for muscle specific delivery. From this list ART-1, CDH15, MUSK, CACNA1S, and SLC2A4 were selected for further validation.

### Example 2: Validation of Muscle Expression and Antibody Accessibility

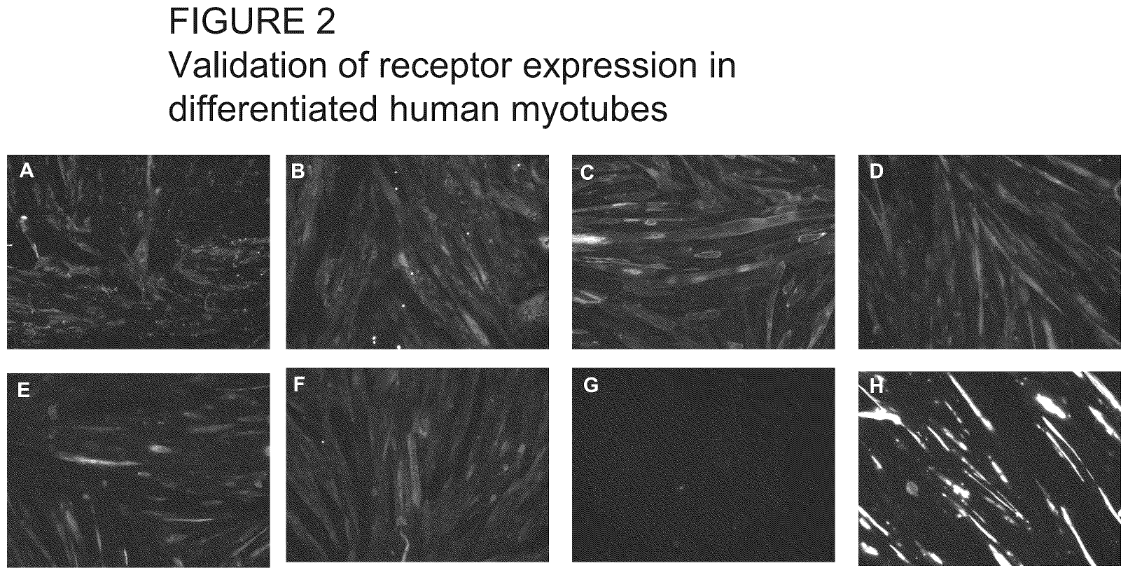

FIG. 2 shows fluorescence microscopy validation of selected receptor expression in differentiated myotubes. Differentiated human myotubes were fixed with 4% PFA and blocked with PBS+5% serum+1% BSA for 1 hour at room temperature. Cells were stained with primary antibodies against A—ALK2, B—GLUT4, C—CDH15, D—MuSK, E—ART1, F—CHRND, G—N/A (no primary antibody only secondary as a negative control) and H—MYHC4 at 5 μg/mL in blocking solution for 1 hour at room temperature or overnight at 4° C. then washed 3× with PBS+0.1% tween20. Next, cells were co-stained with Hoechst 33342 and AF647-conjugated secondary antibody for 1 hour at room temperature. After washing 3× with PBS+0.1% tween 20, cells were imaged for detection on the DAPI and Cy5 channels (shown).

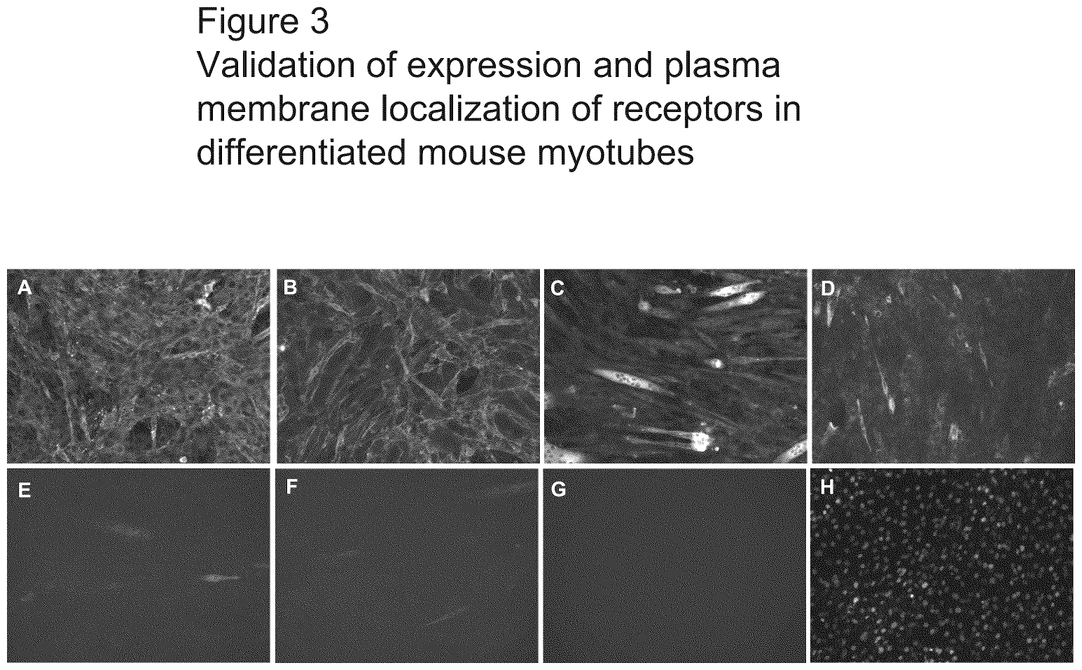

FIG. 3 shows fluorescence microscopy validation of receptor expression in differentiated mouse myotubes. Differentiated C2C12 myotubes were fixed with 4% PFA and blocked with PBS+5% serum+1% BSA for 1 hour at room temperature. Cells were stained with primary antibodies against A—GLUT4, B—CDH15, C—MuSK, D—ART1, E—CHRND, F—ACNA1S, and G—N/A (no primary antibody only secondary as a negative control) at 5 μg/mL in blocking solution for 1 hour at room temperature or overnight at 4° C. then washed 3× with PBS+0.1% tween20. Next, cells were co-stained with Hoechst 33342 and AF647-conjugated secondary antibody for 1 hour at room temperature. After washing 3× with PBS+0.1% tween 20, cells were imaged for detection on the DAPI and Cy5 channels (shown). Panel H is a representative image of the same treatment as panel G but in the DAPI channel to show stained nuclei.

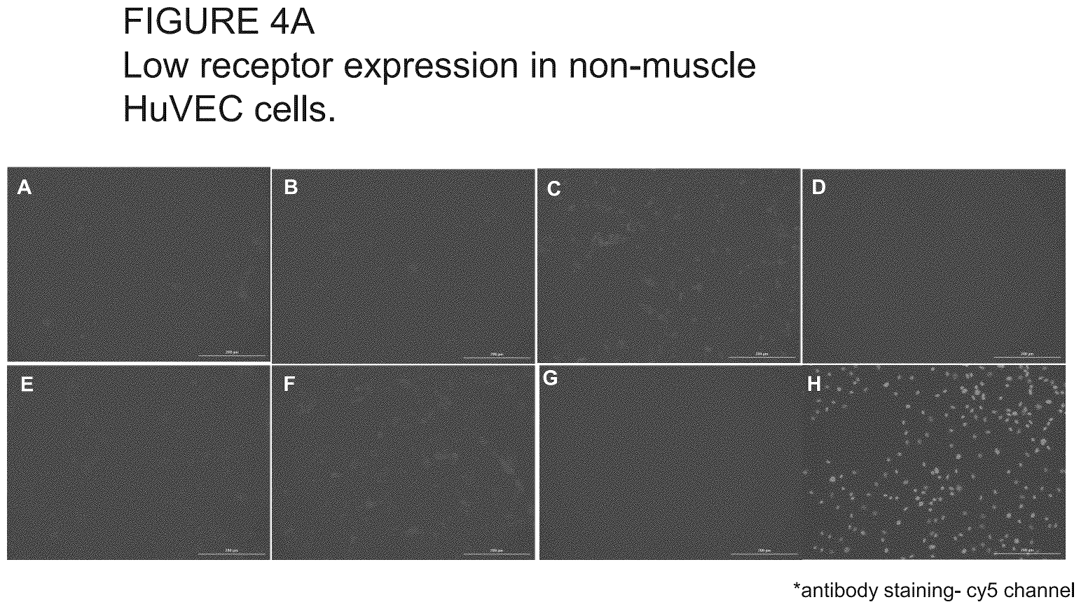

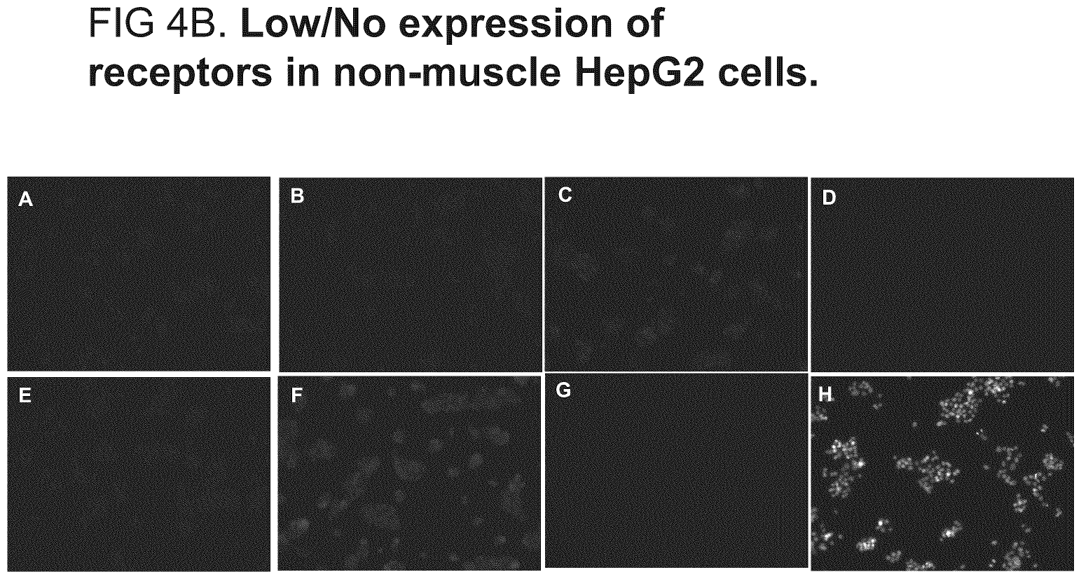

FIGS. 4A-4B show fluorescence microscopy Low/No expression of receptors in non-muscle cells. FIG. 4A shows HuVEC cells fixed with 4% PFA and blocked with PBS+5% serum+1% BSA for 1 hour at room temperature. Cells were stained with primary antibodies against A—ALK2, B—CHRND, C—GLUT4, D—CDH15, E—MuSK, F—ART1, and G—N/A at 2 μg/mL in blocking solution for 1 hour at room temperature or overnight at 4° C. then washed 3× with PBS+0.1% tween20. Next, cells were co-stained with Hoechst 33342 and AF647-conjugated secondary antibody for 1 hour at room temperature. After washing 3× with PBS+0.1% tween 20, cells were imaged for detection on the DAPI and Cy5 (shown) channels. Panel H represents the same treatment as panel G but in the DAPI channel to show stained nuclei.

FIG. 4B shows HepG2 cells fixed with 4% PFA and blocked with PBS+5% serum+1% BSA for 1 hour at room temperature. Cells were stained with primary antibodies against A—ALK2, B—CHRND, C—GLUT4, D—CDH15, E—MuSK, F—ART1, G—N/A at 2 μg/mL in blocking solution for 1 hour at room temperature or overnight at 4° C. then washed 3× with PBS+0.1% tween20. Next, cells were co-stained with Hoechst 33342 and AF647-conjugated secondary antibody for 1 hour at room temperature. After washing 3× with PBS+0.1% tween 20, cells were imaged for detection on the DAPI and Cy5 (shown) channels. Panel H represents the same treatment as panel G but in the DAPI channel to show stained nuclei.

### Example 3: Internalization and Delivery of an Antibody Conjugate to Muscle Cells

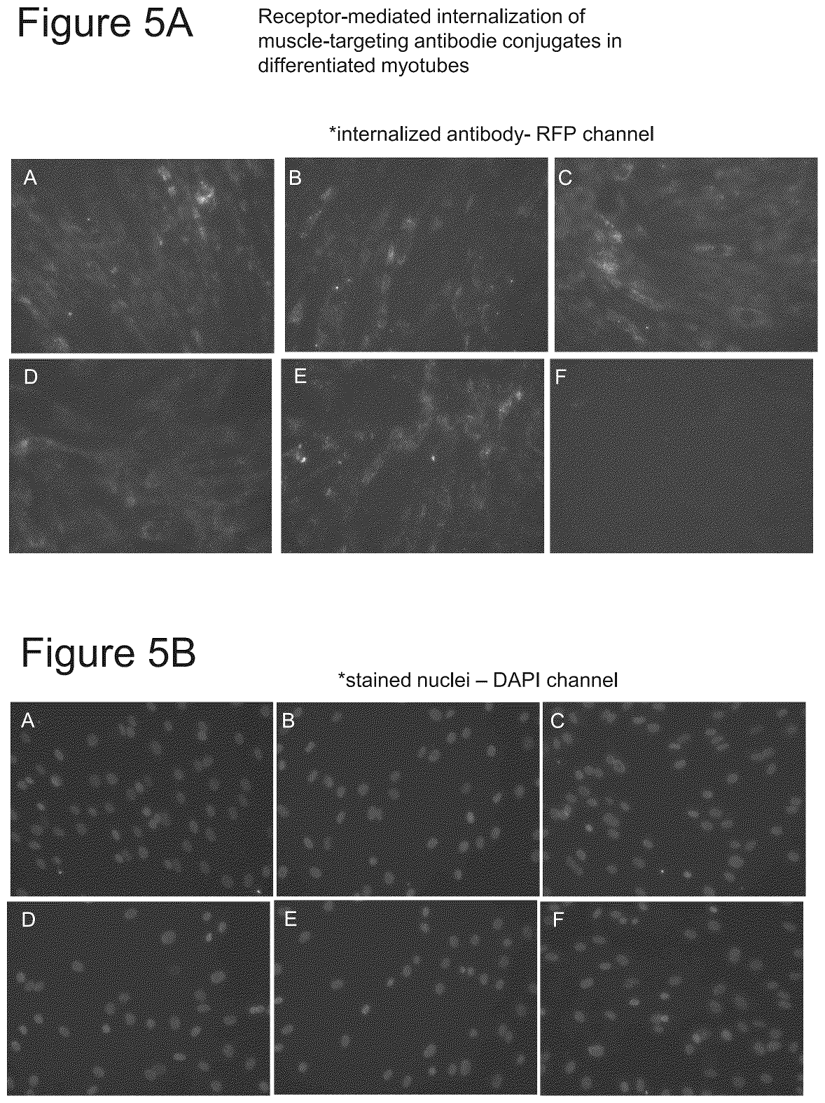

Antibody internalization can be observed by labeling of antibodies with a pH sensitive fluorophore such as pHrodo. pHrodo is non-fluorescent at neutral pH when present in cell culture media or bound the antigen on the cell surface. After cellular internalization into endosomal or lysosomal compartments where a lower pH is present pHrodo becomes fluorescent releasing light in the red spectrum (RFP channel). Thus, cells can be treated with labeled antibody and fluorescence can be observed and quantitated over time by fluorescent microscopy or measurement of fluorescence intensity. This data can be used to determine the time for receptor internalization following antibody binding. To measure recycling of the receptor following treatment with the fluorescent antibody, free antibody is removed and cells are washed, and reagents are added to inhibit protein synthesis and degradation. Then fluorescence intensity over time can be measured to calculate recycling of the receptor. FIGS. 5A-5B show an example of fluorescence microscopy of receptor-mediated antibody internalization for muscle specific receptors. Differentiated human myotubes were treated with antibodies against muscle-expressed receptors labelled with pHrodo dye. Cells were incubated with pHrodo antibody (5 ug/mL) on ice for 1 hour, then washed with PBS to remove unbound antibody. Cells were then incubated at 37° C. to allow for internalization and imaged after an additional 1 hour. Internalized antibody was detected in the RFP channel (shown in 5A) and scored by visual inspection of several wells + low internalization, ++ moderate internalization, +++ high internalization. (A—ALK2 +++, B—GLUT4 ++, C—CDH15 +++, D—MuSK +) Transferrin-pHrodo +++ (panel E) and secondary Ab-pHrodo (panel F) were included as positive and negative controls, respectively.

FIG. 5B shows cell nuclei from the same wells as FIG. 5A (stained with Hoechst 33342) detected in the DAPI channel (blue).

Extensive mining of RNA and protein expression profiles from healthy and diseased tissue uncovered numerous muscle-selective surface antigens whose expression remains stable with disease progression. The surface receptors described here were chosen based on the following criteria:
Plasma membrane localization;
Accessibility via extracellular space;
Expression in muscle tissue;
Selective/enriched expression in muscle compared to other tissue types; and
Timescale for internalization and recycling to the cell surface.

The surface antigens described in this disclosure include ALK2, KLHL41, SLC2A4, FABP3, LMOD2, CDH15, MUSK, ART1, CACNA1S, CLCN1, ENO3, KLHL41, ACTA1, MYMX, CHARNA1, SEMA6C, XIRP1, XIRP2, CFL2, SVIL, and CAVIN4.

Previous attempts to use neutralizing monoclonal antibodies to treat DMD have not resulted in approved therapies (domagrozumab/anti-myostatin/Pfizer). Structural and bioinformatic analysis of the described receptors allowed for identification of sequences useful for antibody development. Developed antibodies were selected based on their ability to bind surface antigen and deliver therapeutic to skeletal muscle without negatively impacting endogenous receptor function.

While exemplary embodiments have been shown and described herein, such embodiments are by way of example only. Numerous variations, changes, and substitutions can be performed on the exemplary embodiments. It should be understood that various alternatives to the embodiments described herein may be employed.

1. A method of delivering an agent to muscle tissue in vivo in a tissue-specific or tissue selective manner, the method comprising contacting the surface of muscle cell(s) of the muscle tissue with an effective amount of an agent that specifically or selective binds a targeted protein expressed on the cell surface of the muscle cell(s) of the muscle tissue;
wherein the targeted protein is enriched in muscle tissue relative to other tissues;
wherein the targeted protein has stable or increased expression in diseased tissue relative to otherwise comparable normal tissue; and
optionally wherein the targeted protein is internalized and recycled within from about 2 minutes to about 12 hours, from about 2 minutes to about 10 hours, from about 2 minutes to about 8 hours, from about 2 minutes to about 6 hours, from about 2 minutes to about 4 hours, from about 2 minutes to about 2 hours, from about 2 minutes to about 60 minutes, from about 2 minutes to about 40 minutes, from about 2 minutes to about 20 minutes, from about 2 minutes to about 10 minutes, from about 10 minutes to about 12 hours, from about 20 minutes to about 12 hours, from about 40 minutes to about 12 hours, from about 60 minutes to about 12 hours, from about 2 hours to about 12 hours, from about 4 hours to about 12 hours, from about 6 hours to about 12 hours, from about 8 hours to about 12 hours, from about 10 hours to about 12 hours, or from about 11 hours to about 12 hours;
thereby delivering an agent to muscle tissue in vivo in a tissue-specific or tissue selective manner.

2. The method of claim 1, wherein:
the targeted protein is expressed from a gene selected from the group consisting of: KLHL41, LMOD2, ENO3, FABP3, CHRNA1, SEMA6C, XIRP2, XIRP1, CAVIN4, CFL2, SVIL, MUSK, ART1, CACNA1S, CDH15, CLCN1, MYMX, and ACTA1;
the agent is a specific or selective binding agent of the targeted protein, optionally wherein the specific or selective binding agent is a soluble receptor or a soluble ligand, optionally wherein specific or selective binding agent is the soluble receptor, and wherein the soluble receptor comprises the extracellular domain of a receptor, optionally wherein the soluble receptor is a Fc fusion protein; and/or
the agent is an antibody or an antigen-binding fragment thereof, optionally wherein the antibody or antigen-binding fragment thereof is selected from the group consisting of a monoclonal antibody, a bispecific antibody, a Fab, a Fab-Fc, a Fv, a single chain Fv (scFv), a diabody, a minibody, a VNAR, and an immunoglobulin single variable domain (ISV).

3-8. (canceled)

9. A method of treating a pathology in an individual, comprising administering to the individual a therapeutically effective amount of a therapeutic targeting agent that specifically or selectively binds a targeted protein expressed on muscle tissue cell surface;
wherein the targeted protein is enriched in muscle tissue relative to other tissues;
wherein the targeted protein has stable or increased expression in diseased tissue relative to otherwise comparable normal tissue; and
optionally wherein the targeted protein is internalized and recycled within from about 2 minutes to about 12 hours, from about 2 minutes to about 10 hours, from about 2 minutes to about 8 hours, from about 2 minutes to about 6 hours, from about 2 minutes to about 4 hours, from about 2 minutes to about 2 hours, from about 2 minutes to about 60 minutes, from about 2 minutes to about 40 minutes, from about 2 minutes to about 20 minutes, from about 2 minutes to about 10 minutes, from about 10 minutes to about 12 hours, from about 20 minutes to about 12 hours, from about 40 minutes to about 12 hours, from about 60 minutes to about 12 hours, from about 2 hours to about 12 hours, from about 4 hours to about 12 hours, from about 6 hours to about 12 hours, from about 8 hours to about 12 hours, from about 10 hours to about 12 hours, or from about 11 hours to about 12 hours;
thereby treating the pathology in the individual.

10. The method of claim 9, wherein the therapeutic targeting agent is an agent that comprises an active agent component and a targeting agent component,
wherein the active agent component is selected from the group consisting of: a radionuclide; a chemotherapeutic agent; an immune stimulatory agent; an anti-neoplastic agent; an anti-inflammatory agent; a pro-inflammatory agent; a pro-apoptotic agent; a pro-coagulant; a toxin; an antibiotic; a hormone; an enzyme; a protein; a carrier protein; a lytic agent; a small molecule; aptamers; cells, vaccine-induced or other immune cells; nanoparticles; transferrins; immunoglobulins; multivalent antibodies; lipids; lipoproteins; liposomes; an altered natural ligand; a gene or nucleic acid; an oligonucleotide; RNA; siRNA; an ncRNA mimic; a short-harpin RNA (shRNA); a dicer-dependent siRNA (di-siRNA); an antisense oligonucleotide (ASO); a gapmer; a mixmer; a double-stranded RNA (dsRNA); a single stranded RNAi (ssRNAi); a DNA-directed RNA interference (ddRNAi); an RNA activating oligonucleotide (RNAa); an aptamer; an exon skipping oligonucleotide; a miRNA; a miRNA mimic; an mRNA; a guide RNA; a viral or non-viral gene delivery vector; a prodrug; and a promolecule, and
wherein the targeting agent component specifically or selectively binds to the targeted protein, optionally wherein the targeting agent component comprises a specific or selective binding agent for the targeted protein, optionally wherein the specific or selective binding agent is a soluble receptor or a soluble ligand, optionally wherein the specific or selective binding agent is a soluble receptor, and wherein the soluble receptor comprises the extracellular domain of a receptor, optionally wherein the soluble receptor is a Fc fusion protein or a biologically active fragment thereof.

11-14. (canceled)

15. The method according to claim 10, wherein the targeting agent component comprises an antibody or an antigen-binding fragment thereof, optionally wherein the antibody or antigen-binding fragment thereof is selected from the group consisting of a monoclonal antibody, a bispecific antibody, a Fab, a Fab-Fc, a Fv, a single chain Fv (scFv), a diabody, a minibody, a VNAR, and an immunoglobulin single variable domain (ISV).

16. (canceled)

17. The method of claim 10, wherein the active agent component is an oligonucleotide or polynucleotide,
wherein the targeting agent component is an antibody or an antigen-binding fragment thereof,
wherein the active agent component is conjugated to the targeting agent component, and
wherein the oligonucleotide targets a disease gene expressed in muscle tissue.

18. The method of claim 9, wherein the targeted protein is encoded by a gene selected from the group consisting of: KLHL41, LMOD2, ENO3, FABP3, CHRNA1, SEMA6C, XIRP2, XIRP1, CAVIN4, CFL2, SVIL, MUSK, ART1, CACNA1S, CDH15, CLCN1, MYMX, and ACTA1.

19. A method of delivering an imaging agent to muscle tissue in a tissue-specific or tissue-selective manner, comprising contacting the surface of muscle cell(s) of the muscle tissue with an effective amount of an imaging agent that comprises an imaging agent component and a targeting agent component, wherein the targeting agent component specifically or selectively binds to a targeted protein expressed on the cell surface of the muscle cells of the muscle tissue;
wherein the targeted protein is enriched in muscle tissue relative to other tissues;
wherein the targeted protein has stable or increased expression in diseased tissue relative to otherwise comparable normal tissue; and
optionally wherein the targeted protein is internalized and recycled within from about 2 minutes to about 12 hours, from about 2 minutes to about 10 hours, from about 2 minutes to about 8 hours, from about 2 minutes to about 6 hours, from about 2 minutes to about 4 hours, from about 2 minutes to about 2 hours, from about 2 minutes to about 60 minutes, from about 2 minutes to about 40 minutes, from about 2 minutes to about 20 minutes, from about 2 minutes to about 10 minutes, from about 10 minutes to about 12 hours, from about 20 minutes to about 12 hours, from about 40 minutes to about 12 hours, from about 60 minutes to about 12 hours, from about 2 hours to about 12 hours, from about 4 hours to about 12 hours, from about 6 hours to about 12 hours, from about 8 hours to about 12 hours, from about 10 hours to about 12 hours, or from about 11 hours to about 12 hours;
thereby delivering an imaging agent to muscle tissue in a tissue-specific or tissue-selective manner

20-24. (canceled)

25. The method of claim 19, wherein:
the targeting agent component is an antibody or an antigen-binding fragment thereof, optionally wherein the antibody or antigen-binding fragment thereof is selected from the group consisting of a monoclonal antibody, a bispecific antibody, a Fab, a Fab-Fc, a Fv, a single chain Fv (scFv), a diabody, a minibody, a VNAR, and an immunoglobulin single variable domain (ISV); and/or
the imaging agent component is selected from the group consisting of a radioactive agent, radioisotope or radiopharmaceutical; a contrast agent; a magnetic agent or a paramagnetic agent; liposomes; ultrasound agents; a gene vector or virus inducing a detecting agent; an enzyme; a prosthetic group; a fluorescent material; a luminescent material; and a bioluminescent material.

26-50. (canceled)

51. The method of claim 9, wherein the pathology comprises a neuromuscular disorder including a muscular dystrophy or a myopathy.

52. The method of claim 51, wherein the pathology is a Duchenne's muscular dystrophy (DMD), Myotonic Dystrophy (MD), Facioscapulohumeral muscular dystrophy (FSHD), Limb-Girdle muscular dystrophy (LGMD), Becker muscular dystrophy, Oculopharyngeal muscular dystrophy, Emery-Dreifuss muscular dystrophy, or Distal muscular dystrophy.

53. The method of claim 1, wherein the individual is a human.

54. The method of claim 1, administering comprises oral, by inhalation, intranasal, by injection, subcutaneous, intramuscular, administering directly to a tissue or call, administering indirectly to a tissue or a cell, intravenously, rectally, intrathecal, intra ocular, in the ear, intraperitoneal, or topical.

55. The method of claim 1, wherein the contacting occurs in an individual, and the contacting results from administering an agent to the individual.

56. The method of claim 1, wherein the assessing comprises a biopsy.

57-61. (canceled)

62. The method of claim 10, wherein the oligonucleotide is a DUX4-targeting antisense oligonucleotide (ASO).
